(19) United States
(12) Patent Application Publication
(10) Pub. No.: US 2025/0000968 A1
(43) Pub. Date: Jan. 2, 2025

(54) SARS-COV-2 SPIKE FUSED TO A HEPATITIS B SURFACE ANTIGEN

(71) Applicant: The United States of America, as represented by the Secretary,Department of Health and Human Service, Bethesda, MD (US)

(72) Inventors: John Mascola, Rockville, MD (US); Cuiping Liu, North Potomac, MD (US); Wei Shi, Rockville, MD (US); Amarendra Pegu, Rockville, MD (US); Lingshu Wang, North Potomac, MD (US); Wing-Pui Kong, Germantown, MD (US)

(73) Assignee: The United States of America, as represented by the Secretary,Department of Health and Human Service, Bethesda, MD (US)

(21) Appl. No.: 18/709,441

(22) PCT Filed: Nov. 11, 2022
(86) PCT No.: PCT/US2022/079750

§ 371 Date: May 10, 2024

Related U.S. Application Data
(60) Provisional application No. 63/278,956, filed on Nov. 12, 2021.

(51) Int. Cl. A61K 39/215; A61K 39/00; A61P 31/14; C07K 14/005

(52) U.S. Cl. CPC A61K 39/215; A61P 31/14; C07K 14/005; A61K 2039/5258; A61K 2039/53; A61K 2039/55; A61K 2039/55555; A61K 2039/575; C07K 2319/735; C12N 2730/10134; C12N 2770/20034

(57) ABSTRACT

Provided herein are nucleic acid molecules encoding a SARS-CoV-2 S ectodomain—HBsAg fusion protein. When expressed in mammalian cells (for example, by administration to a mammalian subject), the fusion protein self-assembles to form a HBsAg protein nanoparticle with SARS-CoV-2 S ectodomain trimers extending radially outward from an outer surface of the HBsAg protein nanoparticle. Thus, in several aspects, the disclosed nucleic acid molecule can be used to generate an immune response to SARS-CoV-2 in a subject.

### CROSS REFERENCE TO RELATED APPLICATION

This application claims priority to U.S. Provisional Application No. 63/278,956, filed Nov. 12, 2021, which is incorporated by reference in its entirety.

### FIELD

This disclosure concerns fusion proteins comprising a SARS-CoV-2 S ectodomain fused to a Hepatitis B Surface Antigen (HBsAg), nucleic acid molecules encoding the fusion proteins, and their use for eliciting an immune response to SARS-CoV-2.

### BACKGROUND

Coronaviruses are enveloped, positive-sense single-stranded RNA viruses. They have the large genomes (26-32 kb) and are phylogenetically divided into four genera (α, β, γ, δ), with betacoronaviruses further subdivided into four lineages (A, B, C, D). Coronaviruses infect a wide range of avian and mammalian species, including humans.

In 2019, a novel coronavirus (later designated SARS-CoV-2 by the World Health Organization) was identified as the causative agent of an outbreak of pneumonia that was later termed COVID-19. As of August 2021, SARS-CoV-2 had infected more than 200 million people worldwide, leading to more than 4 million deaths. Although multiple SARS-CoV-2 vaccines have been fully approved or granted emergency use authorization, a need exists for additional SARS-CoV-2 vaccines that elicit a superior and longer-lasting immune response.

### SUMMARY

Disclosed herein are nucleic acid molecules encoding a fusion protein comprising a recombinant SARS-CoV-2 S ectodomain fused via a peptide linker to a HBsAg protein. In some aspects, provided is a nucleic acid molecule encoding a fusion protein comprising a recombinant SARS-CoV-2 spike(S) ectodomain comprising F817P, A892P, A899P, A942P, K986P, and V987P substitutions (according to the reference SARS-CoV-2 S protein set forth as SEQ ID NO: 1) and an amino acid sequence at least 90% identical to residues 14-1206 of SEQ ID NO: 2 fused via a peptide linker to a HBsAg protein. In some aspects, a S1/S2 protease cleavage site of the SARS-CoV-2 S ectodomain is mutated to inhibit protease cleavage. When expressed in mammalian cells (for example, by administration to a mammalian subject), the fusion protein self-assembles to form a HBsAg nanoparticle with SARS-CoV-2 S ectodomain trimers extending radially outward from an outer surface of the HBsAg nanoparticle.

In some aspects, the fusion protein comprises a SARS-CoV-2 ectodomain with the F817P, A892P, A899P, A942P, K986P, V987P substitutions, and the RRAR (682-685)-to-GSAS substitution according to the reference SARS-CoV-2 S protein set forth as SEQ ID NO: 1, and the fusion protein comprises an amino acid sequence at least 90% identical (such as at least 95% identical, at least 99% identical, or identical) to residues 14 to the C-terminus of any one of SEQ ID NOs: 10-12, 17-18, 57-296, or 315-321.

In additional aspects, a nucleic acid molecule is provided that encodes a fusion protein comprising a recombinant Coronavirus S ectodomain fused via a peptide linker to a HBsAg protein. When expressed in mammalian cells (for example, by administration to a mammalian subject), the fusion protein self-assembles to form a HBsAg nanoparticle with trimers of the Coronavirus S ectodomain extending radially outward from an outer surface of the HBsAg nanoparticle.

The nucleic acid molecule can be any suitable nucleic acid molecule for expression in mammalian cells, such as DNA or RNA, including mRNA, modified mRNA, circular RNA, modified circular RNA, and replication competent RNA with or without modified bases. The nucleic acid molecule can be included in a vector, such as an expression vector.

Additionally provided is a HBsAg nanoparticle encoded by the nucleic acid or vector as described herein, and comprising Coronavirus S ectodomain trimers extending radially outward from an outer surface of the nanoparticle.

Further provided are immunogenic compositions that include the nucleic acid molecule encoding the fusion protein, vector, or HBsAg nanoparticle, and a pharmaceutically acceptable carrier.

Also provided are methods of eliciting an immune response against Coronavirus (e.g., SARS-CoV-2) in a subject and methods of immunizing a subject against Coronavirus (e.g., SARS-CoV-2) infection by administering to the subject an effective amount of the nucleic acid molecule encoding the fusion protein, vector, or HBsAg nanoparticle, disclosed herein.

The foregoing and other objects and features of the disclosure will become more apparent from the following detailed description, which proceeds with reference to the accompanying figures.

### BRIEF DESCRIPTION OF THE DRAWINGS

FIGS. 1A-1D. Self-assembling SARS-CoV-2 S6P—HBsAg nanoparticles. (1A) Schematic representation of full-length SARS-CoV-2 WA1 spike, S2P (1-1273), S2P (1-1206), S6P (1-1206) and S6P—HBsAg fusion protein. The individual domain of the spike or its ectodomains was colored and indicated. S2P (1-1273) is full-length spike harboring K986P, V987P mutations and RRAR-to-GSAS substitution at the furin cleavage site. S2P (1-1206) and S6P (1-1206) represent the ectodomains of S2P and S6P spanning from amino acids 1 to 1206, respectively. S6P (1-1206) contains six proline substitutions: F817P, A892P, A899P, A942P, K986P and V987P. Both S2P (1-1206) and S6P (1-1206) contain GSAS substitution at the furin cleavage site. S6P—HBsAg fusion was constructed by joining S6P (1-1206) and HBsAg from amino acid 1 to 226 with a linker of GS repeats. (1B) Constructs tested for nanoparticle formation on a HBsAg core. The spike-HBsAg constructs for SARS-CoV-2 WA1 spike, S2P and S6P with a 12-GS linker were named S-12-HBsAg, S2P-12-HBsAg and S6P-12-HBsAg, respectively. Varying linkers of 8- to 24-GS were tested and named accordingly. (1C) Negative stain EM images of S6P-8-HBsAg (left), S6P-12-HBsAg (middle) and S6P-16-HBsAg (right) from the 50% sucrose fractions. The insets show 2D class averages of corresponding nanoparticle constructs. Scale bars represent 100 nm in micrographs and 20 nm in 2D class average images. (1D) Human ACE2 binding and antigenicity of S6P—HBsAgs. Plates were coated with 1 μg/ml of SARS-CoV-2 S2P, S6P or S6P—HBsAg nanoparticles. Human ACE2 was assessed at a concentration ranging from 6.1 ng/ml to 100 μg/ml. The mAbs specific to the NTD, SD1, S2 or RBD domain of SARS-CoV-2 spike were tested from 1 μg/ml to 4 μg/ml.

FIGS. 2A-2B. Neutralization potency elicited by SARS-CoV-2 S6P—HBsAgs. (2A) Immunization schema of SARS-CoV-2 DNA constructs. SARS-CoV-2 WA1 S2P (1-1206), S6P (1-1206), S6P-12-HBsAg, S6P-16-HBsAg and S2P (1-1273) DNA matching mRNA-1273 sequence were evaluated. A total of 10, 2 or 0.4 μg of each DNA was injected intramuscularly to two hind legs of BALB/cJ mice at week 0 and 4, excepting S2P (1-1273), which was administer at week 0 and 3. Electroporation was applied following each immunization. Sera were collected 2 weeks post each immunization and followed by a 5- to 10-week interval for mice immunized with S2P (1-1206), S6P (1-1206), S6P-12-HBsAg or S6P-16-HBsAg. (2B) Neutralization ID50s against WA1 pseudovirus. The sera at 2 weeks post the second immunization were assessed. The ID50s were plotted in the box and whiskers format, in which, the median ID50 was indicated by a horizontal line, and the data points in the median quartile of each group were boxed. The error bars represent 95% confidence interval. The immunogens and doses were indicated. The neutralization ID50s were plotted in a logarithmic scale as dots, diamonds and triangles for 10, 2 and 0.4 μg doses, respectively. The geometric mean titers were listed below each group. The statistical analyses were performed using the two-way ANOVA test for S2P (1-1206), S6P (1-1206), S6P-12-HBsAg and S6P-16-HBsAg. The comparison between the same dose of S6P—HBsAg and S2P (1-1273) was done using the two tailed Mann-Whitney test with Dunn's multiple comparisons test. *p<0.05; **p<0.01; ***p<0.001; ****p<0.0001. Full statistical analyses are shown in Table 5.

FIG. 3. Neutralization potency against SARS-CoV-2 variant pseudovriuses elicited by SARS-CoV-2 S6P—HBsAgs. The sera at 2 weeks post the second immunization with SARS-CoV-2 S2P (1-1206), S6P (1-1206), S6P-12-HBsAg or S6P-16-HBsAg were evaluated against SARS-CoV-2 D614G, B.1.351, B.1.617.2 and B.1.1.529 pseudoviruses. The ID50s were plotted in the box and whiskers format. The median ID50 was indicated by a horizontal line, and the data points in the median quartile of each group were boxed. The error bars represent 95% confidence interval. The immunogens and doses were indicated. The ID50s were plotted in a logarithmic scale as dot, diamond or triangle for 10, 2, and 0.4 μg doses, respectively. The geometric mean ID50 for each group was listed below each group. The statistical analyses were performed using the two-way ANOVA test. *p<0.05; **p<0.01.

FIG. 4. Durability of neutralizing antibody responses elicited by SARS-CoV-2 S6P—HBsAgs. The sera from mice immunized with DNA encoding SARS-CoV-2 S2P (1-1206), S6P (1-1206), S6P-12-HBsAg or S6P-16-HBsAg were tested from week 0 to week 45. The ID50s against WA1 pseudovirus were plotted against time. The geometric mean ID50s were shown for week 6 and 14. The ID50 for later time points were obtained from pooled sera for each group. The time courses were shown for the 10, 2 and 0.4 μg doses in different panels.

FIG. 5A-5B. Neutralization potency elicited by SARS-CoV-2 S6P—HBsAgs in mice preimmunized with RECOMBIVAX HB®. (5A) Immunization schema of SARS-CoV-2 constructs. SARS-CoV-2 S2P (1-1273) DNA matching the sequence of mRNA-1273, S6P (1-1206), S6P-12-HBsAg and S6P-16-HBsAg were administered to mice preimmunized with RECOMBIVAX HB®. (5B) Neutralization ID50s against WA1 (left) and B.1.1.529 (BA.1, right) pseudoviruses at week 10. The ID50s were shown in a logarithmic scale in the box and whiskers format. The data points in the median quartile of each group were boxed. The error bars represent 95% confidence interval for the ID50s. The doses and immunogens were indicated. The data from each animal were shown as dots, diamonds and triangles for 10, 2, and 0.4 μg, respectively. The geometric mean ID50 values were indicated. The statistical analyses were performed using the two-way ANOVA test. *p<0.05; ** P<0.01; ***p<0.001; ****p<0.0001.

FIG. 6. Immunoblots of the fractions of SARS-CoV-2 S6P—HBsAgs after ultracentrifugation. Fractions of S6P-8-HBsAg, S6P-12-HBsAg and S6P-16-HBsAg after ultracentrifugation through a sucrose gradient were shown from left to right. Two microliters of each sucrose fraction were spotted onto a nitrocellulose membrane and then blocked with 5% skim milk in phosphate buffered saline-tween (PBST) at room temperature for 1 hour. The membrane was washed with 15 ml PBST solution thrice. Following the washes, the membrane was incubated with 1 μg/ml mAb specific to the NTD, RBD or S2 domain of SARS-CoV-2 spike, in 10 ml of 5% skim milk in PBST at room temperature for 30 min. The membrane was then washed thrice. The horse radish peroxidase (HRP)-conjugated anti-human or anti-mouse secondary antibody ( 1/1000) was incubated with the membrane for 30 min at room temperature. The GE ECL kit was used to develop the membrane. The fractions were shown above the blots. “Top” refers to the layer laid on the top of the 20% sucrose cushion. Numbers denote the percentage of the sucrose solution in a buffer containing 20 mM MES, pH 6.0, 150 mM NaCl. “50b” stands for the band in 50% sucrose after ultracentrifugation. The mAbs S652-118, A23-58.1, LY-CoV555 and WS6, which are specific to the NTD, RBD and S2 domain of SARS-CoV-2 spike, respectively, were used and indicated on the right.

FIGS. 7A-7C. Antibody binding of SARS-CoV-2 S6P—HBsAg nanoparticles. ELISA binding to mAbs specific to different domains of SARS-CoV-2 spike was performed using plates coated with 1 μg/ml SARS-CoV-2 S2P, S6P, S6P-8-HBsAg, S6P-12-HBsAg, or S6P-16-HBsAg. Antibodies were tested from 0.95 pg/ml to 4 μg/ml. Goat anti-human IgG conjugated with horseradish peroxidase ( 1/2000) was used as the secondary antibody. Incubation with primary and secondary antibodies were done at room temperature for 1 h. Plates were developed using TMB at room temperature for 10 min, stopped with IN H₂SO₄. Optical density at 450 nm were measured and plotted against mAb concentrations shown in a logarithmic scale. The classification of the mAbs was indicated on the left for plots in each row.

FIG. 8. IgG titers against SARS-CoV-2 S2P, RBD and NTD. The sera at 2 weeks post the 1^{st} and 2^{nd} immunizations from mice originally naïve to HBsAg were tested. The coating WA1 protein or domains were listed on the left. Endpoint IgG titers were plotted in a logarithmic scale in the box and whiskers format. The data points in the median quartile of each group were boxed. The error bars represent 95% confidence interval. The data for 10, 2 and 0.4 μg doses were plotted as dots, diamonds and triangles, respectively. The doses and immunogens were indicated. Statistical analyses were performed using two-way ANOVA test. *p<0.05; **p<0.01; ***p<0.001; ****p<0.0001.

FIG. 9. Neutralization ID80s against SARS-CoV-2 WA1 and variant pseudoviruses. The sera at 2 weeks post the second immunization were evaluated against SARS-CoV-2 WA1, D614G, B.1.351, B.1.617.2 and B.1.1.529 pseudoviruses. The ID80s were plotted in a logarithmic scale in the box and whiskers format. The data points in the median quartile of each group were boxed. The error bars represent 95% confidence interval. The doses and immunogens were indicated. The data were plotted as dots, diamonds or triangles for 10, 2, and 0.4 μg doses, respectively. The statistical analyses were performed using the two-way ANOVA test. *p<0.05; **p<0.01; ***p<0.001.

FIG. 10. IgG titers against SARS-CoV-2 variant S2Ps. Week 2 (left) and week 6 (right) sera from mice at 2 weeks post the 1st or 2nd DNA immunizations plus electroporation were tested. Endpoint IgG titers were plotted in a logarithmic scale in the box and whiskers format. The data points in the median quartile of each group were boxed. The error bars represent 95% confidence interval. The data for 10, 2 and 0.4 μg doses were shown as dots, diamonds and triangles, respectively. The doses and immunogens were indicated below the plots. Statistical analyses were performed using two-way ANOVA test. *p<0.05; ** p<0.01; ***p<0.001; ****p<0.0001. Less data points were shown for some groups due to the unavailability of the sera for the missing ones. Data points less than 5 in a group were not included in the statistical analyses.

FIG. 11. Neutralization potency at weeks 6 and 14 elicited by SARS-CoV-2 S6P—HBsAgs. Week 6 and 14 sera after two DNA immunizations plus electroporation were tested. WA1 ID50 (top) and ID80s (bottom) were plotted in a logarithmic scale in the box and whiskers format. The data points in the median quartile in each group were boxed. The error bars indicate 95% confidence interval. The data for 10, 2 and 0.4 μg doses were plotted as dots, diamonds and triangles, respectively.

FIG. 12. Durability of anti-spike responses elicited by SARS-CoV-2 S6P—HBsAg. The endpoint IgG titers against WA1 S2P from week 0 to week 45 were shown from left to right for mice immunized twice with S2P (1-1206), S6P (1-1206), S6P-12-HBsAg or S6P-16-HBsAg. 10, 2 and 0.4 μg groups were shown from top to bottom. The endpoint IgG titer from each animal was shown as an individual diamond. The geometric mean titer of each group was shown as a black horizontal bar. Arrows indicate DNA immunizations at week 0 and 4 followed by electroporation.

FIG. 13. Durability of anti-HBsAg responses elicited by SARS-CoV-2 S6P—HBsAg. The endpoint titers against HBsAg from week 0 to week 45 were shown from left to right for mice immunized twice with S2P (1-1206), S6P (1-1206), S6P-12-HBsAg or S6P-16-HBsAg. 10, 2 and 0.4 μg groups were shown from top to bottom. The endpoint IgG titer from each animal was shown as an individual diamond. The geometric mean titer of each group was shown as a black horizontal bar. Arrows indicate DNA immunizations at weeks 0 and 4 followed by electroporation.

FIG. 14. Durability of neutralizing antibody responses elicited by SARS-CoV-2 S6P—HBsAgs. The sera from mice immunized with DNA encoding SARS-CoV-2 S2P (1-1206), S6P (1-1206), S6P-12-HBsAg or S6P-16-HBsAg were tested from week 0 to week 45. The ID80s against WA1 pseudovirus for each group were plotted against time. The geometric mean ID80s were shown for weeks 6 and 14. The ID80 for later time points were obtained from pooled sera for each group. The time courses were shown for the 10, 2 and 0.4 μg doses in different panels.

FIG. 15. Anti-HBsAg endpoint titers in mice preimmunized with RECOMBIVAX HB®. Week 3, 6 and 10 sera from mice preimmunized with RECOMBIVAX HB® followed by one or two DNA immunizations plus electroporation were tested. Endpoint IgG titers were plotted in a logarithmic scale in the box and whiskers format. The data points in the median quartile in each group were boxed. The error bars indicate 95% confidence interval. The data for 10, 2 and 0.4 μg doses were plotted as dots, diamonds and triangles, respectively. The doses and immunogens were indicated. Statistical analyses were performed using the two-way ANOVA test. *p<0.05; **p<0.01; ***p<0.001; ****p<0.0001.

FIG. 16. IgG titers against SARS-CoV-2 WA1 and BA.1 S2Ps in mice preimmunized with RECOMBIVAX HB®. Week 6 and 10 sera from mice preimmunized with RECOMBIVAX HB® followed by one or two DNA immunizations plus electroporation were tested. WA1 and BA.1 S2Ps were used to coat plates and indicated on the top, respectively. Endpoint IgG titers were plotted in a logarithmic scale in the box and whiskers format. The data points in the median quartile of each group were boxed. Error bars represent 95% confidence interval. The data for 10, 2 and 0.4 μg doses were shown as dots, diamonds and triangles, respectively. The doses and immunogens were indicated. Statistical analyses were performed using the two-way ANOVA test. *p<0.05; **p<0.01; ***p<0.001; ****p<0.0001.

FIGS. 17A-17C. Neutralization potency in mice preimmunized with RECOMBIVAX HB®. (17A) ID80s against SARS-CoV-2 WA1 pseudovirus. (17B) ID80s against BA.1 pseudovirus. The sera at 2 weeks post the second DNA immunization plus electroporation from mice preimmunized with RECOMBIVAX HB® were evaluated. The ID80s were plotted in a logarithmic scale in the box and whiskers format. The data points in the median quartile in each group were boxed. Error bars represent 95% confidence interval. The doses and immunogens were indicated. The ID80s were plotted as dots, diamonds and triangles for 10, 2, and 0.4 μg doses, respectively. The statistical analyses were performed using the two-way ANOVA test. *p<0.05; **p<0.01; ***p<0.001; ****p<0.0001. (17C) Neutralization potency against HBV for sera at 2 weeks post the second DNA immunization plus electroporation from mice preimmunized with RECOMBIVAX HB®. Normalized HBV DNA copies to viral control DNA copies in percentage were plotted against serum dilution factors. The data were obtained using pooled sera from each group (n=10). The ID50 and ID80 titers were listed on the right.

FIGS. 18A-18C. Comparison of neutralization potency in mice with and without RECOMBIVAX HB® pre-vaccination. (18A) and (18B) were adopted from FIG. 2; (18C) was from FIG. 5 and FIG. 17A. The data were plotted in a logarithmic scale in the box and whiskers format. The data points in the median quartile in each group were boxed. Error bars represent 95% confidence interval. The doses and immunogens were indicated below the plots. The data points from each animal were shown as dots, diamonds or triangles for 10, 2, and 0.4 μg doses, respectively. The statistical analyses were performed using the two-way ANOVA test. *p<0.05; **p<0.01; ***p<0.001; ****p<0.0001.

### SEQUENCES

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. The Sequence Listing is submitted as an XML file in the form of the file named “106861-02 Sequence Listing” (1,007,616 bytes), which was created on Nov. 10, 2022, which is incorporated by reference herein.

### DETAILED DESCRIPTION

### I. Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Adjuvant: A component of an immunogenic composition used to enhance antigenicity. In some aspects, an adjuvant can include a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). In some aspects, the adjuvant used in a disclosed immunogenic composition is a combination of lecithin and carbomer homopolymer (such as the ADJUPLEX™ adjuvant available from Advanced BioAdjuvants, LLC; see also Wegmann, Clin Vaccine Immunol 22 (9): 1004-1012, 2015). Additional adjuvants for use in the disclosed immunogenic compositions include the QS21 purified plant extract, Matrix M, AS01, MF59, and ALFQ adjuvants. Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL and toll-like receptor (TLR) agonists, such as TLR-9 agonists. The person of ordinary skill in the art is familiar with adjuvants (see, e.g., Singh (ed.) Vaccine Adjuvants and Delivery Systems. Wiley-Interscience, 2007).

Administration: The introduction of an agent, such as a nucleic acid molecule encoding a SARS-CoV-2 S ectodomain-HBsAg fusion protein as disclosed herein, into a subject by a chosen route. Administration can be local or systemic. For example, if the chosen route is intranasal, the agent is administered by introducing the composition into the nasal passages of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in a polypeptide with a different amino acid.

Carrier: An immunogenic molecule to which an antigen can be linked. When linked to a carrier, the antigen may become more immunogenic. Carriers are chosen to increase the immunogenicity of the antigen and/or to elicit antibodies against the carrier which are diagnostically, analytically, and/or therapeutically beneficial. Useful carriers include polymeric carriers, which can be natural (for example, proteins from bacteria or viruses), semi-synthetic or synthetic materials containing one or more functional groups to which a reactant moiety can be attached.

Conservative variants: “Conservative” amino acid substitutions are those substitutions that do not substantially affect or decrease a function of a protein, such as the ability of the protein to induce an immune response when administered to a subject. The term conservative variation also includes the use of a substituted amino acid in place of an unsubstituted parent amino acid. Furthermore, deletions or additions which alter, add or delete a single amino acid or a small percentage of amino acids (for instance less than 5%, in some aspects less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the SARS-CoV-2 S ectodomain-HBsAg fusion protein, such as the ability to self-assemble or induce an immune response when administered to a subject. For instance, if an amino acid residue is essential for a function of the protein, even an otherwise conservative substitution may disrupt that activity. Thus, a conservative substitution does not alter the basic function of a protein of interest.

Control: A reference standard. In some aspects, the control is a negative control sample obtained from a healthy patient. In other aspects, the control is a positive control sample obtained from a patient diagnosed with a Coronavirus infection, such as a SARS-CoV-2 infection. In still other aspects, the control is a historical control or standard reference value or range of values (such as a previously tested control sample, such as a group of SARS-CoV-2 patients with a known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Coronavirus: A large family of positive-sense, single-stranded RNA viruses that can infect humans and non-human animals. Coronaviruses get their name from the crown-like spikes on their surface. The viral envelope is comprised of a lipid bilayer containing the viral membrane (M), envelope (E) and spike(S) proteins. Most coronaviruses cause mild to moderate upper respiratory tract illness, such as the common cold. However, three coronaviruses have emerged that can cause more serious illness and death: severe acute respiratory syndrome coronavirus (SARS-CoV or SARS-CoV-1), SARS-CoV-2, and Middle East respiratory syndrome coronavirus (MERS-CoV). Other coronaviruses that infect humans include human coronavirus 229E (229E-CoV), human coronavirus NL63 (NL63-CoV), human coronavirus OC43 (OC43-CoV), and human coronavirus HKU1 (HKU1-CoV).

Coronavirus Disease 2019 (COVID-19): A disease caused by SARS-CoV-2 infection. Common symptoms include fever, cough, fatigue, shortness of breath or breathing difficulties, and loss of smell and taste. The incubation period may range from one to fourteen days. While most patients have mild symptoms, some develop severe COVID-19 disease, often characterized by acute respiratory distress syndrome (ARDS) that is precipitated by cytokine storm, multi-organ failure, septic shock, and blood clots, and often requiring hospitalization and possible ventilation-based breathing assistance.

A host of underlying medical conditions are known to lead to increased risk of COVID-19, and severe COVID-19, following infection with SARS-CoV-2. Non-limiting examples include heart disease, cancer, chronic obstructive pulmonary disease, type 2 diabetes, type 1 diabetes, obesity, chronic kidney disease, sickle cell disease, asthma, liver disease, chronic lung disease, high blood pressure, or a suppressed immune system due to medical treatment, infection with a pathogen other than SARS-CoV-2, or an autoimmune disorder.

The World Health Organization (WHO) has published testing guidelines for COVID-19 diagnosis (see, e.g., Laboratory Guidelines for the Detection and Diagnosis of COVID-19 virus infection, July 2020). The standard method of testing for SARS-CoV-2 infection is real-time reverse transcription polymerase chain reaction (rRT-PCR) on respiratory samples obtained by a nasopharyngeal swab. Standard diagnostic methods of the detection of symptoms of COVID-19 are also utilized (e.g., lung inflammation, shortness of breath, low oxygen saturation, etc).

Degenerate variant: A polynucleotide encoding a polypeptide that includes a sequence that is degenerate as a result of the genetic code. There are 20 natural amino acids, most of which are specified by more than one codon. Therefore, all degenerate nucleotide sequences are included as long as the amino acid sequence of the polypeptide is unchanged.

Effective amount: An amount of agent, such as a nucleic acid molecule encoding a Coronavirus S ectodomain—HBsAg fusion protein (for example, a SARS-CoV-2 S ectodomain—HBsAg fusion protein) as described herein, that is sufficient to elicit a desired response, such as an immune response in a subject. It is understood that to obtain a protective immune response against an antigen of interest can require multiple administrations of a disclosed immunogen, and/or administration of a disclosed immunogen as the “prime” in a prime boost protocol wherein the boost immunogen can be different from the prime immunogen. Accordingly, an effective amount of a disclosed immunogen can be the amount of the immunogen sufficient to elicit a priming immune response in a subject that can be subsequently boosted with the same or a different immunogen to elicit a protective immune response.

In one example, a desired response is to inhibit or reduce or prevent SARS-CoV-2 infection. The SARS-CoV-2 infection does not need to be completely eliminated or reduced or prevented for the method to be effective. For example, administration of an effective amount of the agent can induce an immune response that decreases the SARS-CoV-2 infection (for example, as measured by infection of cells, or by number or percentage of subjects infected by the SARS-CoV-2) by a desired amount, for example by at least 50%, at least 60%, at least 70%, at least 80%, at least 90%, at least 95%, at least 98%, or even at least 100% (elimination or prevention of detectable SARS-CoV-2 infection), as compared to a suitable control.

Expression: Transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, stop codons, 5′ and 3′ UTRs, a Poly A tail, etc. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, pLac, pTRP, pTAC (ptrp-lac hybrid promoter) and the like may be used. In one aspect, when cloning in mammalian cell systems, promoters derived from the genome of mammalian cells (such as metallothionein promoter, EF1α promoter) or from mammalian viruses (such as the cytomegalovirus (CMV) promoter, the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Fusion Protein: A single polypeptide chain including the sequence of two or more heterologous proteins, often linked by a peptide linker.

Hepatitis B surface antigen (HBsAg): A Hepatitis B virus glycoprotein that forms an outer layer surrounding the viral nucleocapsid in native virions, which are approximately 30-42 nm in diameter. In native virions, HBsAg is expressed as three different proteins originating at different start codons along a single open reading frame, resulting in Large (L), Middle (M), and Small(S) surface antigen proteins. Isoforms of HBsAg differ by the number of domains. S has only the S domain, while M has both a Pre-S2 and an S domain, and L has a Pre-S1 domain in addition to the Pre-S2 and S domain. Recombinant HBsAg nanoparticles form by expression of the L, M, or S proteins alone or in combination in eukaryotic systems. Recombinant nanoparticles consisting of the HBsAg S protein alone are observed to have a diameter of about 22 nm and octahedral symmetry. Described herein is the fusion of a heterologous protein (SARS-CoV-2 S ectodomain) to the N-terminus of a HBsAg protein (such as a HBsAg S, HBsAg M, or HBsAg L protein) to form a HBsAg nanoparticle displaying the SARS-CoV-2 S ectodomain on the surface of the nanoparticle.

Heterologous: Originating from a separate genetic source or species. For example, a heterologous polypeptide or polynucleotide refers to a polypeptide or polynucleotide derived from a different source or species.

Host cells: Cells in which a vector can be propagated and its DNA can be expressed. The cell may be prokaryotic or eukaryotic. The term also includes any progeny of the subject host cell. It is understood that all progenies may not be identical to the parental cell since there may be mutations that occur during replication. However, such progenies are included when the term “host cell” is used.

Immune response: A response of a cell of the immune system, such as a B cell, T cell, or monocyte, to a stimulus. In some aspects, the response is specific for a particular antigen (an “antigen-specific response”), such as a SARS-CoV-2 spike protein. In some aspects, the immune response is a T cell response, such as a CD4+ response or a CD8+ response. In other aspects, the response is a B cell response, and results in the production of specific antibodies. “Priming an immune response” refers to treatment of a subject with a “prime” immunogen/immunogenic composition to induce an immune response that is subsequently “boosted” with a boost immunogen/immunogenic composition. Together, the prime and boost immunizations produce the desired immune response in the subject.

Immunogenic composition: A composition that includes a nucleic acid molecule encoding a Coronavirus S ectodomain-HBsAg fusion protein (such as a SARS-CoV-2 S ectodomain-HBsAg fusion protein) as described herein that elicits a measurable Cytotoxic T lymphocytes (CTL) response against the Coronavirus S ectodomain (such as SARS-CoV-2 S ectodomain), and/or elicits a measurable B cell response (such as production of antibodies) against the Coronavirus S ectodomain (such as SARS-CoV-2 S ectodomain), when administered to a subject. For in vivo use, the immunogenic composition can include the nucleic acid molecule in a pharmaceutically acceptable carrier and may also include other agents, such as an adjuvant.

Isolated: An “isolated” biological component has been substantially separated or purified away from other biological components, such as other biological components in which the component naturally occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% isolated, such as at least 75%, 80%, 90%, 95%, 98%, 99%, or even 99.9% isolated.

Nucleic acid molecule: A polymeric form of nucleotides, which may include both sense and anti-sense strands of RNA, mRNA, circular RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, an mRNA, or a circular RNA to serve as templates for synthesis of other polymers and macromolecules in biological processes having either a defined sequence of nucleotides (i.e., rRNA, tRNA and mRNA) or a defined sequence of amino acids and the biological properties resulting therefrom.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens (such as a nucleic acid molecule encoding a SARS-CoV-2 S ectodomain-HBsAg fusion protein) and immunogenic compositions containing the immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate. In particular aspects, suitable for administration to a subject the carrier may be sterile, and/or suspended or otherwise contained in a unit dosage form containing one or more measured doses of the composition suitable to elicit the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in viral load. “Ameliorating” refers to the reduction in the number or severity of signs or symptoms of a disease, such as a coronavirus infection.

Recombinant: A recombinant nucleic acid, vector or virus is one that has a sequence that is not naturally occurring or has a sequence that is made by an artificial combination of two otherwise separated segments of sequence. This artificial combination can be accomplished, for example, by the artificial manipulation of isolated segments of nucleic acids, for example, using genetic engineering techniques.

SARS-CoV-2: A positive-sense, single stranded RNA virus of the genus betacoronavirus that has emerged as a highly fatal cause of severe acute respiratory infection. The viral genome is capped, polyadenylated, and covered with nucleocapsid proteins. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike(S)-envelope (E)-membrane (M) and nucleocapsid (N)-3′. Symptoms of SARS-CoV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

Standard methods for detecting viral infection may be used to detect SARS-CoV-2 infection, including but not limited to, assessment of patient symptoms and background and genetic tests such as reverse transcription-polymerase chain reaction (rRT-PCR). The test can be done on patient samples such as respiratory or blood samples.

SARS-CoV-2 Spike(S): A class I fusion glycoprotein initially synthesized as a precursor protein of approximately 1273 amino acids in size. Individual precursor S polypeptides form a homotrimer and undergo glycosylation within the Golgi apparatus as well as processing to remove the signal peptide. The S polypeptide includes S1 and S2 proteins separated by a protease cleavage site between approximately position 685/686. Cleavage at this site generates separate S1 and S2 polypeptide chains, which remain associated as S1/S2 protomers within the homotrimer. It is believed that the beta coronaviruses are generally not cleaved prior to the low pH cleavage that occurs in the late endosome-early lysosome by the TMPRSS2 protease, at the start of the fusion peptide. The S1 subunit is distal to the virus membrane and contains the receptor-binding domain (RBD) that is believed to mediate virus attachment to its host receptor. The S2 subunit is believed to contain the fusion protein machinery, such as the fusion peptide, two heptad-repeat sequences (HR1 and HR2) and a central helix typical of fusion glycoproteins, a transmembrane domain, and the cytosolic tail domain.

Unless context indicates otherwise, the numbering used in the disclosed SARS-CoV-2 S proteins and fragments thereof is relative to the S protein of SARS-CoV-2, the sequence of which is provided as SEQ ID NO: 1, and deposited as NCBI Ref. No. YP_009724390.1, which is incorporated by reference herein in its entirety.

Sequence identity: The similarity between amino acid or nucleotide sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide or polynucleotide will possess a relatively high degree of sequence identity when aligned using standard methods.

Methods of alignment of sequences for comparison are known. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Variants of a polypeptide or nucleic acid sequence are typically characterized by possession of at least about 75%, for example, at least about 80%, 85%, 90%, 91%, 92%, 93%, 94%, 95%, 96%, 97%, 98% or 99% sequence identity counted over the full-length alignment with the amino acid or nucleotide sequence of interest. Sequences with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids (or 30-60 nucleotides), and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

Signal Peptide: A short amino acid sequence (e.g., approximately 10-35 amino acids in length) that directs newly synthesized secretory or membrane proteins to and through membranes (for example, the endoplasmic reticulum membrane). Signal peptides are typically located at the N-terminus of a polypeptide and are removed by signal peptidases. Signal peptide sequences typically contain three common structural features: an N-terminal polar basic region (n-region), a hydrophobic core, and a hydrophilic c-region).

Single chain SARS-CoV-2 S ectodomain: A recombinant SARS-CoV-2 S ectodomain including the SARS-CoV-2 S1 and S2 domains in a single contiguous polypeptide chain. Single chain SARS-CoV-2 S ectodomain can trimerize to form a SARS-CoV-2 S ectodomain trimer. A single SARS-CoV-2 S ectodomain provided herein includes mutations to prevent protease cleavage at the S1/S2 cleavage site. Therefore, when produced in cells, the SARS-CoV-2 S polypeptide harboring such a mutated furin cleavage site is not cleaved into separate S1 and S2 polypeptide chains.

Subject: Living multicellular vertebrate organisms, a category that includes human and non-human mammals. In some aspects, the subject is a human. In some examples, a subject who is in need of inhibiting or preventing a SARS-CoV-2 infection is selected. For example, the subject can be uninfected and at risk of SARS-CoV-2 infection.

Vaccine: A pharmaceutical composition that elicits a prophylactic or therapeutic immune response in a subject. In some cases, the immune response is a protective immune response. Typically, a vaccine elicits an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In one specific, non-limiting example, a vaccine reduces the severity of the symptoms associated with SARS-CoV-2 infection and/or decreases the viral load compared to a control. In another non-limiting example, a vaccine reduces SARS-CoV-2 infection and/or transmission compared to a control.

Vector: An entity containing a DNA or RNA molecule bearing a promoter(s) that is operationally linked to the coding sequence of a protein (such as an immunogenic protein) of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses. Non-limiting examples of viral vectors include adenovirus vectors (such as Ad26, Ad5, or ChAdOx1 recombinant vector, adeno-associated virus (AAV) vectors, and poxvirus vectors (e.g., vaccinia, fowlpox).

### II. Nucleic Acid Molecules Encoding Coronavirus SARS-CoV-2 S Ectodomain—HBsAg Fusion Proteins

### A. Nucleic Acid Molecules Encoding SARS-CoV-2 S Ectodomain—HBsAg Fusion Proteins

Disclosed herein are nucleic acid molecules encoding a fusion protein comprising a recombinant SARS-CoV-2 S ectodomain fused via a peptide linker to a HBsAg protein. When expressed in mammalian cells (for example, by administration to a mammalian subject), the fusion protein self-assembles to form a HBsAg nanoparticle with SARS-CoV-2 S ectodomain trimers extending radially outward from an outer surface of the HBsAg nanoparticle. The SARS-CoV-2 S ectodomain include one or more amino acid substitutions that stabilize the ectodomain trimer in its prefusion conformation.

An exemplary sequence of native SARS-CoV-2 S protein (including the native ectodomain and TM and CT domains) is provided as SEQ ID NO: 1 (NCBI Ref. No. YP_009724390.1, incorporated by reference herein):

MFVFLVLLPLVSSQCVNLTTRTQLPPAYTNSFTRGVYYPDKVERSSVLHSTQDLFLPFFSNVTWFHAIHVSG
TNGTKRFDNPVLPENDGVYFASTEKSNIIRGWIFGTTLDSKTQSLLIVNNAINVVIKVCEFQFCNDPFLGVY
YHKNNKSWMESEFRVYSSANNCTFEYVSQPFLMDLEGKQGNEKNLREFVEKNIDGYFKIYSKHTPINLVRDL
PQGFSALEPLVDLPIGINITRFQTLLALHRSYLTPGDSSSGWTAGAAAYYVGYLQPRTELLKYNENGTITDA
VDCALDPLSETKCTLKSFTVEKGIYQTSNERVQPTESIVREPNIINLCPFGEVENATRFASVYAWNRKRISN
CVADYSVLYNSASESTFKCYGVSPTKLNDLCFTNVYADSFVIRGDEVRQIAPGQTGKIADYNYKLPDDETGC
VIAWNSNNLDSKVGGNYNYLYRLERKSNLKPFERDISTEIYQAGSTPCNGVEGENCYFPLQSYGFQPINGVG
YQPYRVVVLSFELLHAPATVCGPKKSTNLVKNKCVNFNFNGLIGTGVLTESNKKELPFQQFGRDIADTIDAV
RDPQTLEILDITPCSFGGVSVITPGINTSNQVAVLYQDVNCTEVPVAIHADQLTPTWRVYSTGSNVFQTRAG
CLIGAEHVNNSYECDIPIGAGICASYQTQTNSPRRARSVASQSIIAYTMSLGAENSVAYSNNSIAIPTNFTI
SVTTEILPVSMTKISVDCTMYICGDSTECSNLLLQYGSFCTQLNRALTGIAVEQDKNTQEVFAQVKQIYKTP
PIKDFGGFNFSQILPDPSKPSKRSFIEDLLENKVTLADAGFIKQYGDCLGDIAARDLICAQKENGLIVLPPL
LIDEMIAQYISALLAGTITSGWTFGAGAALQIPFAMQMAYRFNGIGVTQNVLYENQKLIANQFNSAIGKIQD
SLSSTASALGKLQDVVNQNAQALNTLVKQLSSNFGAISSVLNDILSRLDKVEAEVQIDRLITGRLQSLQTYV
TQQLIRAAEIRASANLAATKMSECVLGQSKRVDFCGKGYHLMSFPQSAPHGVVFLHVTYVPAQEKNETTAPA
ICHDGKAHFPREGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT

The amino acid numbering used herein for residues of the SARS-CoV-2 S protein is with reference to the SARS-CoV-2 S sequence provided as SEQ ID NO: 1. With reference to the SARS-CoV-2 S protein sequence provided as SEQ ID NO: 1, the ectodomain of the SARS-CoV-2 S protein includes about residues 14-1206. Residues 1-13 are the signal peptide, which is removed during cellular processing. The S1/S2 cleavage site is located at position 685/686. The HR1 is located at about residues 915-983. The central helix is located at about residues 988-1029. The HR2 is located at about 1162-1194. The C-terminal end of the S2 ectodomain is located at about residue 1208. In some aspects, the protomers of the prefusion-stabilized SARS-CoV-2 S ectodomain trimer can have a C-terminal residue of the C-terminal residue of the HR2 (e.g., position 1194), or the ectodomain (e.g., position 1208), or from one of positions 1194-1208. The position numbering of the S protein may vary between SARS-CoV-2 stains, but the sequences can be aligned to determine relevant structural domains and cleavage sites. It will be appreciated that a few residues (such as up to 10) on the N- and/or C-terminal ends of the ectodomain can be removed or modified in the disclosed immunogens without decreasing the utility of the S ectodomain as an immunogen.

The recombinant SARS-CoV-2 S ectodomain included in the fusion protein is stabilized in a prefusion conformation by one or more amino acid substitutions. In some aspects, the recombinant SARS-CoV-2 S ectodomain included in the fusion protein is stabilized in a prefusion conformation by the “6P” mutations: F817P, A892P, A899P, A942P, K986P, and V987P relative to SEQ ID NO: 1.

In some aspects, the recombinant SARS-CoV-2 S ectodomain included in the fusion protein is a single-chain SARS-CoV-2 S ectodomain including a mutation of the S1/S2 protease cleavage site to prevent cleavage and formation of distinct S1 and S2 polypeptide chains. In some aspects, the S1 and S2 polypeptides in the single chain SARS-CoV-2 S ectodomain are joined by a linker, such as a peptide linker. Examples of peptide linkers that can be used include glycine, serine, and glycine-serine linkers. In some aspects, the S1/S2 protease cleavage site is mutated by a RRAR (682-685)-to-GSAS substitution. Any of the prefusion stabilizing mutations (or combinations thereof) disclosed herein can be included in the single chain SARS-CoV-2 S ectodomain.

An exemplary sequence of a recombinant SARS-CoV-2 S protein including the 6P substitutions for prefusion stabilization for inclusion in the fusion protein is provided as SEQ ID NO: 2:

MFVFLVLLPLVSSQCVNLTTRTQLPPAYTNSFTRGVYYPDKVFRSSVLHSTQDLFLPFFSNVTWFHAIHVSG
TNGTKRFDNPVLPENDGVYFASTEKSNIIRGWIFGTTLDSKTQSLLIVNNAINVVIKVCEFQFCNDPFLGVY
YHKNNKSWMESEFRVYSSANNCTFEYVSQPFLMDLEGKQGNEKNLREFVEKNIDGYFKIYSKHTPINLVRDL
PQGESALEPLVDLPIGINITRFQTLLALHRSYLTPGDSSSGWTAGAAAYYVGYLQPRTELLKYNENGTITDA
VDCALDPLSETKCTLKSFTVEKGIYQTSNERVQPTESIVREPNIINLCPFGEVENATRFASVYAWNRKRISN
CVADYSVLYNSASFSTFKCYGVSPTKLNDLCFINVYADSFVIRGDEVRQIAPGQTGKIADYNYKLPDDETGC
VIAWNSNNLDSKVGGNYNYLYRLERKSNLKPFERDISTEIYQAGSTPCNGVEGENCYFPLQSYGFQPINGVG
YQPYRVVVLSFELLHAPATVCGPKKSTNLVKNKCVNFNENGLIGIGVLIESNKKELPFQQFGRDIADTTDAV
RDPQTLEILDITPCSFGGVSVITPGINTSNQVAVLYQDVNCTEVPVAIHADQLTPTWRVYSTGSNVFQTRAG
CLIGAEHVNNSYECDIPIGAGICASYQTQTNSPRRARSVASQSIIAYTMSLGAENSVAYSNNSIAIPINFTI
SVTTEILPVSMTKTSVDCTMYICGDSTECSNLLLQYGSFCTQLNRALTGIAVEQDKNTQEVFAQVKQIYKTP
PIKDFGGFNFSQILPDPSKPSKRSPIEDLLENKVTLADAGFIKQYGDCLGDIAARDLICAQKENGLIVLPPL
LIDEMIAQYTSALLAGTITSGWTFGAGPALQIPFPMQMAYRENGIGVTQNVLYENQKLIANQFNSAIGKIQD
SLSSTPSALGKLQDVVNQNAQALNTLVKQLSSNFGAISSVLNDILSRLDPPEAEVQIDRLITGRLQSLQTYV
TQQLIRAAEIRASANLAATKMSECVLGQSKRVDFCGKGYHLMSFPQSAPHGVVFLHVTYVPAQEKNETTAPA
ICHDGKAHFPREGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYIKWPWYIWLGFIAGL
IAIVMVTIMLCCMTSCCSCLKGCCSCGSCCKEDEDDSEPVLKGVKLHYT

An exemplary sequence of a recombinant SARS-CoV-2 S protein including the 6P substitutions for prefusion stabilization and a RRAR (682-685)-to-GSAS substitution for removal of the S1/S2 protease cleavage site for inclusion in the fusion protein is provided as SEQ ID NO: 3:

MFVFLVLLPLVSSQCVNLITRTQLPPAYTNSFTRGVYYPDKVFRSSVLHSTQDLFLPFFSNVTWFHAIHVSG
TNGTKRFDNPVLPENDGVYFASTEKSNIIRGWIFGTTLDSKTQSLLIVNNAINVVIKVCEFQFCNDPFLGVY
YHKNNKSWMESEFRVYSSANNCTFEYVSQPFLMDLEGKQGNEKNLREFVEKNIDGYFKIYSKHTPINLVRDL
PQGFSALEPLVDLPIGINITRFQTLLALHRSYLTPGDSSSGWTAGAAAYYVGYLQPRTELLKYNENGTITDA
VDCALDPLSETKCTLKSFTVEKGIYQTSNERVQPTESIVREPNIINLCPFGEVENATRFASVYAWNRKRISN
CVADYSVLYNSASFSTFKCYGVSPTKLNDLCFTNVYADSFVIRGDEVRQIAPGQTGKIADYNYKLPDDETGC
VIAWNSNNLDSKVGGNYNYLYRLERKSNLKPFERDISTEIYQAGSTPCNGVEGENCYFPLQSYGFQPINGVG
YQPYRVVVLSFELLHAPATVCGPKKSTNLVKNKCVNFNFNGLIGTGVLTESNKKFLPFQQFGRDIADTTDAV
RDPQTLEILDITPCSFGGVSVITPGINTSNQVAVLYQDVNCTEVPVAIHADQLTPTWRVYSTGSNVFQTRAG
CLIGAEHVNNSYECDIPIGAGICASYQTQTNSPGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTP
PIKDFGGFNFSQILPDPSKPSKRSPIEDLLENKVTLADAGFIKQYGDCLGDIAARDLICAQKENGLTVLPPL
LTDEMIAQYTSALLAGTITSGWTFGAGPALQIPFPMQMAYRENGIGVTQNVLYENQKLIANQFNSAIGKIQD
SLSSTPSALGKLQDVVNQNAQALNTLVKQLSSNEGAISSVLNDILSRLDPPEAEVQIDRLITGRLQSLQTYV
TQQLIRAAEIRASANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT

In some aspects, the recombinant SARS-CoV-2 S ectodomain included in the fusion protein is a spike protein ectodomain from a SARS-CoV-2 variant, such as the ‘delta’ variant, that has been modified to include the 6P substation and/or a modification to remove the S1/S2 protease cleavage site (such as RRAR (682-685)-to-GSAS). For example, the recombinant SARS-CoV-2 S ectodomain included in the fusion protein may include substitutions relative to SEQ ID NO: 1 that are present in variant spike protein sequences, such as K417N, L452R, T478K, E484K, E484Q, N501Y, and/or P681R substitutions. In some aspects, the recombinant SARS-CoV-2 S ectodomain included in the fusion protein comprises an N501Y substitution, a E484K substitution, N501Y, K417N, and E484K substitutions, L452R, T478K, and P681R substitutions, or L452R, E484Q, and P681R substitutions.

In some aspects, the recombinant SARS-CoV-2 S ectodomain included in the fusion protein comprises F817P, A892P, A899P, A942P, K986P, and V987P substitutions (numbering with reference to SEQ ID NO: 1) and an amino acid sequence at least 90% (such as at least 95%, at least 96%, at least 97%, at least 98%, or at least 99%) identical to residues 14-1206 of SEQ ID NO: 3. In some aspects, the recombinant SARS-CoV-2 S ectodomain included in the fusion protein comprises or consists of residues 14-1206 of SEQ ID NO: 3.

In some aspects, the recombinant SARS-CoV-2 S ectodomain included in the fusion protein comprises F817P, A892P, A899P, A942P, K986P, V987P, and RRAR (682-685)-to-GSAS substitutions (numbering with reference to SEQ ID NO: 1) and an amino acid sequence at least 90% (such as at least 95%, at least 96%, at least 97%, at least 98%, or at least 99%) identical to residues 14-1206 of SEQ ID NO: 3. In some aspects, the recombinant SARS-CoV-2 S ectodomain included in the fusion protein comprises or consists of residues 14-1206 of SEQ ID NO: 3.

Exemplary sequences of additional recombinant SARS-CoV-2 S proteins including the 6P substitutions, and a RRAR (682-685)-to-GSAS substitution for removal of the S1/S2 protease cleavage site for inclusion in the fusion protein are provided as SEQ ID NOs: 19-44:

>B.1.617.2 (Based on GISAID ID: EPI_ISL_2020950),
SEQ ID NO: 19
MFVFLVLLPLVSSQCVNLRTRTQLPPAYTNSFTRGVYYPDKVFRSSVLHSTQDLFLPFFSNVTWFHAIHVSGTNGTKRFD
NPVLPFNDGVYFASTEKSNIIRGWIFGTTLDSKTQSLLIVNNATNVVIKVCEFQFCNDPFLDVYYHKNNKSWMESGVYSS
ANNCTFEYVSQPFLMDLEGKQGNFKNLREFVFKNIDGYFKIYSKHTPINLVRDLPQGFSALEPLVDLPIGINITRFQTLL
ALHRSYLTPGDSSSGWTAGAAAYYVGYLQPRTFLLKYNENGTITDAVDCALDPLSETKCTLKSFTVEKGIYQTSNERVQP
TESIVRFPNITNLCPFGEVENATRFASVYAWNRKRISNCVADYSVLYNSASFSTFKCYGVSPTKLNDLCFTNVYADSFVI
RGDEVRQIAPGQTGKIADYNYKLPDDFTGCVIAWNSNNLDSKVGGNYNYRYRLFRKSNLKPFERDISTEIYQAGSKPCNG
VEGFNCYFPLQSYGFQPTNGVGYQPYRVVVLSFELLHAPATVCGPKKSTNLVKNKCVNFNFNGLTGTGVLTESNKKFLPF
QQFGRDIADTTDAVRDPQTLEILDITPCSFGGVSVITPGTNTSNQVAVLYQGVNCTEVPVAIHADQLTPTWRVYSTGSNV
FQTRAGCLIGAEHVNNSYECDIPIGAGICASYQTQTNSRGSASSVASQSIIAYTMSLGAENSVAYSNNSIAIPTNFTISV
TTEILPVSMTKTSVDCTMYICGDSTECSNLLLQYGSFCTQLNRALTGIAVEQDKNTQEVFAQVKQIYKTPPIKDEGGENF
SQILPDPSKPSKRSPIEDLLENKVTLADAGFIKQYGDCLGDIAARDLICAQKENGLTVLPPLLTDEMIAQYTSALLAGTI
TSGWTFGAGPALQIPFPMQMAYRENGIGVTQNVLYENQKLIANQFNSAIGKIQDSLSSTPSALGKLQNVVNQNAQALNTL
VKQLSSNFGAISSVLNDILSRLDPPEAEVQIDRLITGRLQSLQTYVTQQLIRAAEIRASANLAATKMSECVLGQSKRVDF
CGKGYHLMSFPQSAPHGVVFLHVTYVPAQEKNFTTAPAICHDGKAHFPREGVFVSNGTHWFVTQRNFYEPQIITTDNTFV
SGNCDVVIGIVNNTVYDPLQPELDSFKEELDKYFKNHTSPDVDLGDISGINASVVNIQKEIDRLNEVAKNLNESLIDLQE
LGKYEQYIKWPWYIWLGFIAGLIAIVMVTIMLCCMTSCCSCLKGCCSCGSCCKFDEDDSEPVLKGVKLHYT
>B.1.617.2 (Based on CDC website on Aug. 31, 2021),
SEQ ID NO: 20
MFVFLVLLPLVSSQCVNLRTRTQLPPAYTNSFTRGVYYPDKVFRSSVLHSTQDLFLPFFSNVTWFHAIHFSGTNGTKRED
NPVLPFNDGVYFASIEKSNIIRGWIFGTTLDSKTQSLLIVNNATNVVIKVCEFQFCNDPFLDVYYHKNNKSWMESGVYSS
ANNCTFEYVSQPFLMDLEGKQGNFKNLREFVFKNIDGYFKIYSKHTPINLVRDLPQGFSVLEPLVDLPIGINITRFQTLL
ALHRSYLTPGDSSSGLTAGAAAYYVGYLQPRTFLLKYNENGTITDAVDCALDPLSETKCTLKSFTVEKGIYQTSNERVQP
TESIVRFPNITNLCPFGEVENATRFASVYAWNRKRISNCVADYSVLYNSASFSTFKCYGVSPTKLNDLCFTNVYADSFVI
RGDEVRQIAPGQTGNIADYNYKLPDDFTGCVIAWNSNNLDSKVGGNYNYRYRLFRKSNLKPFERDISTEIYQAGSKPCNG
VEGFNCYFPLQSYGFQPTNGVGYQPYRVVVLSFELLHAPATVCGPKKSTNLVKNKCVNFNFNGLIGTGVLTESNKKELPF
QQFGRDIADTTDAVRDPQTLEILDITPCSFGGVSVITPGTNTSNQVAVLYQGVNCTEVPVAIHADQLTPTWRVYSTGSNV
FQTRAGCLIGAEHVNNSYECDIPIGAGICASYQTQTNSRGSASSVASQSIIAYTMSLGAENSVAYSNNSIAIPTNFTISV
TTEILPVSMTKTSVDCTMYICGDSTECSNLLLQYGSFCTQLNRALTGIAVEQDKNTQEVFAQVKQIYKTPPIKDEGGENF
SQILPDPSKPSKRSPIEDLLENKVTLADAGFIKQYGDCLGDIAARDLICAQKENGLTVLPPLLTDEMIAQYTSALLAGTI
TSGWTFGAGPALQIPFPMQMAYRFNGIGVTQNVLYENQKLIANQFNSAIGKIQDSLSSTPSALGKLQNVVNQNAQALNTL
VKQLSSNFGAISSVLNDILSRLDPPEAEVQIDRLITGRLQSLQTYVTQQLIRAAEIRASANLAATKMSECVLGQSKRVDF
CGKGYHLMSFPQSAPHGVVFLHVTYVPAQEKNFTTAPAICHDGKAHFPREGVFVSNGTHWEVTQRNFYEPQIITTDNTFV
SGNCDVVIGIVNNTVYDPLQPELDSFKEELDKYFKNHTSPDVDLGDISGINASVVNIQKEIDRLNEVAKNLNESLIDLQE
LGKYEQYIKWPWYIWLGFIAGLIAIVMVTIMLCCMTSCCSCLKGCCSCGSCCKFDEDDSEPVLKGVKLHYT
>B.1.1.7 (Based on GISAID ID: EPI_ISL_629440),
SEQ ID NO: 21
MFVFLVLLPLVSSQCVNLTTRTQLPPAYTNSFTRGVYYPDKVFRSSVLHSTQDLFLPFFSNVTWFHAISGTNGTKRFDNP
VLPFNDGVYFASTEKSNIIRGWIFGTTLDSKTQSLLIVNNATNVVIKVCEFQFCNDPFLGVYHKNNKSWMESEFRVYSSA
NNCTFEYVSQPFLMDLEGKQGNFKNLREFVFKNIDGYFKIYSKHTPINLVRDLPQGFSALEPLVDLPIGINITRFQTLLA
LHRSYLTPGDSSSGWTAGAAAYYVGYLQPRTFLLKYNENGTITDAVDCALDPLSETKCTLKSFTVEKGIYQTSNERVQPT
ESIVRFPNITNLCPFGEVENATRFASVYAWNRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLYRLFRKSNLKPFERDISTEIYQAGSTPCNGV
EGFNCYFPLQSYGFQPTYGVGYQPYRVVVLSFELLHAPATVCGPKKSTNLVKNKCVNFNFNGLTGTGVLTESNKKELPFQ
QFGRDIDDTTDAVRDPQTLEILDITPCSFGGVSVITPGTNTSNQVAVLYQGVNCTEVPVAIHADQLTPTWRVYSTGSNVF
QTRAGCLIGAEHVNNSYECDIPIGAGICASYQTQTNSHGSASSVASQSIIAYTMSLGAENSVAYSNNSIAIPINFTISVT
TEILPVSMTKTSVDCTMYICGDSTECSNLLLQYGSFCTQLNRALTGIAVEQDKNTQEVFAQVKQIYKTPPIKDFGGFNFS
QILPDPSKPSKRSPIEDLLFNKVTLADAGFIKQYGDCLGDIAARDLICAQKFNGLTVLPPLLTDEMIAQYTSALLAGTIT
SGWTFGAGPALQIPFPMQMAYRENGIGVTQNVLYENQKLIANQFNSAIGKIQDSLSSTPSALGKLQDVVNQNAQALNTLV
KQLSSNFGAISSVLNDILARLDPPEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNGTHWFVTQRNFYEPQIITTHNTFVS
GNCDVVIGIVNNTVYDPLQPELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEPVLKGVKLHYT
>B.1.1.7 (Based on CDC website on 08/31/2021),
SEQ ID NO: 22
MFVFLVLLPLVSSQCVNLTTRTQLPPAYTNSFTRGVYYPDKVFRSSVLHSTQDLFLPFFSNVTWFHAISGTNGTKRFDNP
VLPFNDGVYFASTEKSNIIRGWIFGTTLDSKTQSLLIVNNATNVVIKVCEFQFCNDPFLGVYHKNNKSWMESEFRVYSSA
NNCTFEYVSQPFLMDLEGKQGNFKNLREFVEKNIDGYFKIYSKHTPINLVRDLPQGFSALEPLVDLPIGINITRFQTLLA
LHRSYLTPGDSSSGWTAGAAAYYVGYLQPRTFLLKYNENGTITDAVDCALDPLSETKCTLKSFTVEKGIYQTSNERVQPT
ESIVRFPNITNLCPFGEVENATRFASVYAWNRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLYRLFRKSNLKPFERDISTEIYQAGSTPCNGV
KGFNCYFPLQPYGFQPTYGVGYQPYRVVVLSFELLHAPATVCGPKKSTNLVKNKCVNFNFNGLTGTGVLTESNKKELPFQ
QFGRDIDDTTDAVRDPQTLEILDITPCSFGGVSVITPGTNTSNQVAVLYQGVNCTEVPVAIHADQLTPTWRVYSTGSNVE
QTRAGCLIGAEHVNNSYECDIPIGAGICASYQTQTNSHGSASSVASQSIIAYTMSLGAENSVAYSNNSIAIPINFTISVT
TEILPVSMTKTSVDCTMYICGDSTECSNLLLQYGSFCTQLNRALTGIAVEQDKNTQEVFAQVKQTYKTPPIKDFGGENES
QILPDPSKPSKRSPIEDLLFNKVTLADAGFIKQYGDCLGDIAARDLICAQKFNGLTVLPPLLTDEMIAQYTSALLAGTIT
SGWTFGAGPALQIPFPMQMAYRFNGIGVTQNVLYENQKLIANQFNSAIGKIQDSLSSTPSALGKLQDVVNQNAQALNTLV
KQLSSNFGAISSVLNDILARLDPPEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNGTHWFVTQRNFYEPQIITTHNTFVS
GNCDVVIGIVNNTVYDPLQPELDSFKEELDKYFKNHTSPDVDLGDISGINASVVNIQKEIDRLNEVANNLNESLIDLQEL
GKYEQYIKWPWYIWLGFIAGLIAIVMVTIMLCCMTSCCSCLKGCCSCGSCCKFDEDDSEPVLKGVKLHYT
>B.1.351 (Based on GISAID ID: EPI_ISL_1120737),
SEQ ID NO: 23
MFVFLVLLPLVSSQCVNFTTRTQLPPAYTNSFTRGVYYPDKVFRSSVLHSTQDLFLPFFSNVTWFHAIHVSGTNGTKRFA
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRGLPQGFSALEPLVDLPIGINITRFQT
LHRSYLTPGDSSSGWTAGAAAYYVGYLQPRTELLKYNENGTITDAVDCALDPLSETKCTLKSFTVEKGIYQTSNERVQPT
ESIVRFPNITNLCPFGEVENATRFASVYAWNRKRISNCVADYSVLYNSASFSTFKCYGVSPTKLNDLCFTNVYADSFVIR
GDEVRQIAPGQTGNIADYNYKLPDDFTGCVIAWNSNNLDSKVGGNYNYLYRLFRKSNLKPFERDISTEIYQAGSTPCNGV
KGFNCYFPLQSYGFQPTYGVGYQPYRVVVLSFELLHAPATVCGPKKSTNLVKNKCVNFNFNGLTGTGVLTESNKKELPFQ
QFGRDIADTTDAVRDPQTLEILDITPCSFGGVSVITPGTNTSNQVAVLYQGVNCTEVPVAIHADQLTPTWRVYSTGSNVE
QTRAGCLIGAEHVNNSYECDIPIGAGICASYQTQTNSPGSASSVASQSIIAYTMSLGVENSVAYSNNSIAIPTNFTISVT
TEILPVSMTKTSVDCTMYICGDSTECSNLLLQYGSFCTQLNRALTGIAVEQDKNTQEVFAQVKQIYKTPPIKDFGGFNFS
QILPDPSKPSKRSPIEDLLFNKVTLADAGFIKQYGDCLGDIAARDLICAQKFNGLTVLPPLLTDEMIAQYTSALLAGTIT
SGWTFGAGPALQIPFPMQMAYRFNGIGVTQNVLYENQKLIANQFNSAIGKIQDSLSSTPSALGKLQDVVNQNAQALNTLV
KQLSSNFGAISSVLNDILSRLDPPEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNGTHWFVTQRNFYEPQIITTDNTFVS
GNCDVVIGIVNNTVYDPLQPELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEPVLKGVKLHYT
>P.1 (Based on GISAID ID: EPI_ISL_833171),
SEQ ID NO: 24
MFVFLVLLPLVSSQCVNFTNRTQLPSAYTNSFTRGVYYPDKVFRSSVLHSTQDLFLPFFSNVTWFHAIHVSGTNGTKRED
NPVLPFNDGVYFASTEKSNIIRGWIFGTTLDSKTQSLLIVNNATNVVIKVCEFQFCNYPFLGVYYHKNNKSWMESEFRVY
SSANNCTFEYVSQPFLMDLEGKQGNFKNLSEFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNFRV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGTIADYNYKLPDDFTGCVIAWNSNNLDSKVGGNYNYLYRLFRKSNLKPFERDISTEIYQAGSTPC
NGVKGFNCYFPLQSYGFQPTYGVGYQPYRVVVLSFELLHAPATVCGPKKSTNLVKNKCVNFNFNGLTGTGVLTESNKKFL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEYVNNSYECDIPIGAGICASYQTQTNSPGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSPIEDLLFNKVTLADAGFIKQYGDCLGDIAARDLICAQKFNGLTVLPPLLTDEMIAQYTSALLAG
TITSGWTFGAGPALQIPFPMQMAYRFNGIGVTQNVLYENQKLIANQFNSAIGKIQDSLSSTPSALGKLQDVVNQNAQALN
TLVKQLSSNFGAISSVLNDILSRLDPPEAEVQIDRLITGRLQSLQTYVTQQLIRAAEIRASANLAAIKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASFVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
>B.1.617.1 (Based on GISAID ID: EPI_ISL_1384866),
SEQ ID NO: 25
MFVFLVLLPLVSSQCVNLTTRTQLPPAYTNSFTRGVYYPDKVFRSSVLHSTQDLFLPFFSNVTWFHAIHVSGTNGTKRED
NPVLPFNDGVYFASIEKSNIIRGWIFGTTLDSKTQSLLIVNNATNVVIKVCEFQFCNDPFLDVYYHKNNKSWMKSEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNERV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYRYRLFRKSNLKPFERDISTEIYQAGSTPC
NGVQGFNCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSRGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSPIEDLLENKVTLADAGFIKQYGDCLGDIAARDLICAQKENGLTVLPPLLTDEMIAQYTSALLAG
TITSGWTFGAGPALQIPFPMQMAYRENGIGVTQNVLYENQKLIANQFNSAIGKIQDSLSSTPSALGKLQDVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAHEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
>C.37-1 (Based on GISAID ID: EPI_ISL_1138413),
SEQ ID NO: 26
MFVFLVLLPLVSSQCVNLTTRTQLPPAYTNSFTRGVYYPDKVFRSSVLHSTQDLFLPFFSNVTWFHAIHVSGTNVIKRED
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHNSSSGWTAGAAAYYVGYLQPRTELLKYNENGTITDAVDCALDPLSETKCTLKSFTVEKGIYQTSNERVQPTESIV
RFPNITNLCPFGEVENATRFASVYAWNRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYQYRLERKSNLKPFERDISTEIYQAGSTPCNGVEGEN
CYSPLQSYGFQPTNGVGYQPYRVVVLSFELLHAPATVCGPKKSTNLVKNKCVNFNFNGLTGTGVLTESNKKELPFQQFGR
DIADTTDAVRDPQTLEILDITPCSFGGVSVITPGTNTSNQVAVLYQGVNCTEVPVAIHADQLTPTWRVYSTGSNVFQTRA
GCLIGAEHVNNSYECDIPIGAGICASYQTQTNSPGSASSVASQSIIAYTMSLGAENSVAYSNNSIAIPTNFTISVTTEIL
PVSMTKTSVDCTMYICGDSTECSNLLLQYGSFCTQLNRALTGIAVEQDKNTQEVFAQVKQIYKTPPIKDFGGFNFSQILP
DPSKPSKRSPIEDLLFNKVTLADAGFIKQYGDCLGDIAARDLICAQKFNGLNVLPPLLTDEMIAQYTSALLAGTITSGWT
FGAGPALQIPFPMQMAYRENGIGVTQNVLYENQKLIANQFNSAIGKIQDSLSSTPSALGKLQDVVNQNAQALNTLVKQLS
SNFGAISSVLNDILSRLDPPEAEVQIDRLITGRLQSLQTYVTQQLIRAAEIRASANLAATKMSECVLGQSKRVDFCGKGY
HLMSFPQSAPHGVVFLHVTYVPAQEKNFTTAPAICHDGKAHFPREGVFVSNGTHWFVTQRNFYEPQIITTDNTFVSGNCD
VVIGIVNNTVYDPLQPELDSFKEELDKYFKNHTSPDVDLGDISGINASVVNIQKEIDRLNEVAKNLNESLIDLQELGKYE
QYIKWPWYIWLGFIAGLIAIVMVTIMLCCMTSCCSCLKGCCSCGSCCKFDEDDSEPVLKGVKLHYT
>C.37-2 (Based on GISAID ID: EPI_ISL_1138413),
SEQ ID NO: 27
MFVFLVLLPLVSSQCVNLTTRTQLPPAYTNSFTRGVYYPDKVFRSSVLHSTQDLFLPFFSNVTWFHAIHVSGTNVIKRFD
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHSSSGWTAGAAAYYVGYLQPRTFLLKYNENGTITDAVDCALDPLSETKCTLKSFTVEKGIYQTSNERVQPTESIVR
FPNITNLCPFGEVENATRFASVYAWNRKRISNCVADYSVLYNSASFSTFKCYGVSPTKLNDLCFINVYADSFVIRGDEVR
QIAPGQTGKIADYNYKLPDDFTGCVIAWNSNNLDSKVGGNYNYQYRLFRKSNLKPFERDISTEIYQAGSTPCNGVEGENC
YSPLQSYGFQPTNGVGYQPYRVVVLSFELLHAPATVCGPKKSTNLVKNKCVNFNFNGLTGTGVLTESNKKFLPFQQFGRD
IADTTDAVRDPQTLEILDITPCSFGGVSVITPGTNTSNQVAVLYQGVNCTEVPVAIHADQLTPTWRVYSTGSNVFQTRAG
CLIGAEHVNNSYECDIPIGAGICASYQTQTNSPGSASSVASQSIIAYTMSLGAENSVAYSNNSIAIPTNFTISVTTEILP
VSMTKTSVDCTMYICGDSTECSNLLLQYGSFCTQLNRALTGIAVEQDKNTQEVFAQVKQIYKTPPIKDFGGFNFSQILPD
PSKPSKRSPIEDLLENKVTLADAGFIKQYGDCLGDIAARDLICAQKENGLNVLPPLLTDEMIAQYTSALLAGTITSGWTF
GAGPALQIPFPMQMAYRENGIGVTQNVLYENQKLIANQFNSAIGKIQDSLSSTPSALGKLQDVVNQNAQALNTLVKQLSS
NFGAISSVLNDILSRLDPPEAEVQIDRLITGRLQSLQTYVTQQLIRAAEIRASANLAATKMSECVLGQSKRVDFCGKGYH
LMSFPQSAPHGVVFLHVTYVPAQEKNFTTAPAICHDGKAHFPREGVFVSNGTHWFVTQRNFYEPQIITTDNTFVSGNCDV
VIGIVNNTVYDPLQPELDSFKEELDKYFKNHTSPDVDLGDISGINASVVNIQKEIDRLNEVAKNLNESLIDLQELGKYEQ
YIKWPWYIWLGFIAGLIAIVMVTIMLCCMTSCCSCLKGCCSCGSCCKFDEDDSEPVLKGVKLHYT
>R.1 (Based on GISAID ID: EPI_ISL_1123466),
SEQ ID NO: 28
MFVFLVLLPLVSSQCVNLTTRTQLPPAYTNSFTRGVYYPDKVFRSSVLHSTQDLFLPFFSNVTWFHAIHVSGTNGTKRED
NPVLPFNDGVYFASTEKSNIIRGWIFGTTLDSKTQSLLIVNNATNVVIKVCEFQFCNDPFLGVYYHKNNKSLMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTELLKYNENGTITDAVDCALDPLSETKCTLKSFTVEKGIYQTSNERV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGSTPC
NGVKGFNCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSPGSASSVASQSIIAYTMSLGAENSVAYSNNSIAIPTNFTI
SVTTEILPVSMTKTSVDCTMYICGDSTECSNLLLQYGSFCTQLNRALTVIAVEQDKNTQEVFAQVKQIYKTPPIKDFGGF
NFSQILPDPSKPSKRSPIEDLLFNKVTLADAGFIKQYGDCLGDIAARDLICAQKFNGLTVLPPLLTDEMIAQYTSALLAG
TITSGWTFGAGPALQIPFPMQMAYRFNGIGVTQNVLYENQKLIANQFNSAIGKIQDSLSSTPSALGKLQDVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
>B.1.1.519 (Based on GISAID ID: EPI_ISL_876555) ,
SEQ ID NO: 29
MFVFLVLLPLVSSQCVNLTTRTQLPPAYTNSFTRGVYYPDKVFRSSVLHSTQDLFLPFFSNVTWFHAIHVSGTNGTKRFD
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNERV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGSKPC
NGVEGFNCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSHGSASSVASQSIIAYTMSLGAENSVAYSNNSIAIPTNFTI
SVTTEILPVSMAKTSVDCTMYICGDSTECSNLLLQYGSFCTQLNRALTGIAVEQDKNTQEVFAQVKQIYKTPPIKDFGGF
NFSQILPDPSKPSKRSPIEDLLFNKVTLADAGFIKQYGDCLGDIAARDLICAQKFNGLTVLPPLLTDEMIAQYTSALLAG
TITSGWTFGAGPALQIPFPMQMAYRENGIGVTQNVLYENQKLIANQFNSAIGKIQDSLSSTPSALGKLQDVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
>B.1.525 (Based on GISAID ID: EPI_ISL_760883),
SEQ ID NO: 30
MFVFLVLLPLVSSQCVNLTTRTQLPPAYTNSFTRGVYYPDKVFRSSVLHSTRDLFLPFFSNVTWFHVISGTNGTKRFDNP
VLPFNDGVYFASTEKSNIIRGWIFGTTLDSKTQSLLIVNNATNVVIKVCEFQFCNDPFLGVYHKNNKSWMESEFRVYSSA
NNCTFEYVSQPFLMDLEGKQGNFKNLREFVFKNIDGYFKIYSKHTPINLVRDLPQGFSALEPLVDLPIGINITRFQTLLA
LHRSYLTPGDSSSGWTAGAAAYYVGYLQPRTELLKYNENGTITDAVDCALDPLSETKCTLKSFTVEKGIYQTSNERVQPT
ESIVRFPNITNLCPFGEVENATRFASVYAWNRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLYRLFRKSNLKPFERDISTEIYQAGSTPCNGV
KGFNCYFPLQSYGFQPTNGVGYQPYRVVVLSFELLHAPATVCGPKKSTNLVKNKCVNFNFNGLTGTGVLTESNKKELPFQ
QFGRDIADTTDAVRDPQTLEILDITPCSFGGVSVITPGTNTSNQVAVLYQGVNCTEVPVAIHADQLTPTWRVYSTGSNVF
QTRAGCLIGAEHVNNSYECDIPIGAGICASYQTHINSPGSASSVASQSIIAYTMSLGAENSVAYSNNSIAIPTNFTISVT
TEILPVSMTKTSVDCTMYICGDSTECSNLLLQYGSFCTQLNRALTGIAVEQDKNTQEVFAQVKQTYKTPPIKDFGGFNFS
QILPDPSKPSKRSPIEDLLENKVTLADAGFIKQYGDCLGDIAARDLICAQKFNGLTVLPPLLTDEMIAQYTSALLAGTIT
SGWTLGAGPALQIPFPMQMAYRFNGIGVTQNVLYENQKLIANQFNSAIGKIQDSLSSTPSALGKLQDVVNQNAQALNTLV
KQLSSNFGAISSVLNDILSRLDPPEAEVQIDRLITGRLQSLQTYVTQQLIRAAEIRASANLAATKMSECVLGQSKRVDEC
GKGYHLMSFPQSAPHGVVFLHVTYVPAQEKNFTTAPAICHDGKAHFPREGVFVSNGTHWFVTQRNFYEPQIITTDNTFVS
GNCDVVIGIVNNTVYDPLQPELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEPVLKGVKLHYT
>B.1.526 (Based on GISAID ID: EPI_ISL_1080798),
SEQ ID NO: 31
MFVFFVLLPLVSSQCVNLTTRTQLPPAYTNSFTRGVYYPDKVFRSSVLHSTQDLFLPFFSNVTWFHAIHVSGTNGTKRED
NPVLPFNDGVYFASIEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGGSSSGWTAGAAAYYVGYLQPRTFLLKYNENGTITDAVDCALDPLSETKCTLKSFTVEKGIYQTSNERV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGSTPC
NGVKGFNCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSPGSASSVASQSIIAYTMSLGVENSVAYSNNSIAIPTNFTI
SVTTEILPVSMTKTSVDCTMYICGDSTECSNLLLQYGSFCTQLNRALTGIAVEQDKNTQEVFAQVKQTYKTPPIKDFGGF
NFSQILPDPSKPSKRSPIEDLLFNKVTLADAGFIKQYGDCLGDIAARDLICAQKFNGLTVLPPLLTDEMIAQYTSALLAG
TITSGWTFGAGPALQIPFPMQMAYRENGIGVTQNVLYENQKLIANQFNSAIGKIQDSLSSTPSALGKLQDVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
>B.1.427/429 (Based on GISAID ID: EPI_ISL_984563 (B.1.427), EPI_ISL_672374
(B.1.429)),
SEQ ID NO: 32
MFVFLVLLPLVSIQCVNLTTRTQLPPAYTNSFTRGVYYPDKVFRSSVLHSTQDLFLPFFSNVTWFHAIHVSGTNGTKRED
NPVLPFNDGVYFASTEKSNIIRGWIFGTTLDSKTQSLLIVNNATNVVIKVCEFQFCNDPFLGVYYHKNNKSCMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNERV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYRYRLFRKSNLKPFERDISTEIYQAGSTPC
NGVEGENCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSPGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSPIEDLLFNKVTLADAGFIKQYGDCLGDIAARDLICAQKENGLTVLPPLLTDEMIAQYTSALLAG
TITSGWTFGAGPALQIPFPMQMAYRFNGIGVTQNVLYENQKLIANQFNSAIGKIQDSLSSTPSALGKLQDVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
>B.1.526.1 (Based on GISAID ID: EPI_ISL_896306),
SEQ ID NO: 33
MFVFLVLLPLVSSQCVNLTTRTQLPPAYTNSFTRGVYYPDKVFRSSVLHSTQDLFLPFFSNVTWFHAIHVSGTNGTKRFG
NPVLPFNDGVYFASTEKSNIIRGWIFGTTLDSKTQSLLIVNNATNVVIKVCEFQFCNDPFLGVYHKNNKSWMESESRVYS
SANNCTFEYVSQPFLMDLEGKQGNFKNLREFVFKNIDGYFKIYSKHTPINLVRDLPQGFSALEPLVDLPIGINITRFQTL
LALHRSYLTPGDSSSGWTAGAAAYYVGYLQPRTFLLKYNENGTITDAVDCALDPLSETKCTLKSFTVEKGIYQTSNERVQ
PTESIVRFPNITNLCPFGEVENATRFASVYAWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNYRYRLFRKSNLKPFERDISTEIYQAGSTPCN
GVEGFNCYFPLQSYGFQPTNGVGYQPYRVVVLSFELLHAPATVCGPKKSTNLVKNKCVNFNFNGLTGTGVLTESNKKFLP
FQQFGRDIADTTDAVRDPQTLEILDITPCSFGGVSVITPGTNTSNQVAVLYQGVNCTEVPVAIHADQLTPTWRVYSTGSN
VFQTRAGCLIGAEHVNNSYECDIPIGAGICASYQTQTNSPGSASSVASQSIIAYTMSLGAENSVAYSNNSIAIPTNFTIS
VTTEILPVSMTKTSVDCTMYICGDSTECSNLLLQYGSFCTQLNRALTGIAVEQDKNTQEVFAQVKQIYKTPPIKDFGGEN
FSQILPDPSKPSKRSPIEDLLFNKVTLADAGFIKQYGDCLGDIAARDLICAQKFNGLNVLPPLLTDEMIAQYTSALLAGT
ITSGWTFGAGPALQIPFPMQMAYRENGIGVTQNVLYENQKLIANQFNSAIGKIQDSLSSTPSALGKLQHVVNQNAQALNT
LVKQLSSNFGAISSVLNDILSRLDPPEAEVQIDRLITGRLQSLQTYVTQQLIRAAEIRASANLAATKMSECVLGQSKRVD
FCGKGYHLMSFPQSAPHGVVFLHVTYVPAQEKNFTTAPAICHDGKAHFPREGVFVSNGTHWFVTQRNFYEPQIITTDNTF
VSGNCDVVIGIVNNTVYDPLQPELDSFKEELDKYFKNHTSPDVDLGDISGINASVVNIQKEIDRLNEVAKNLNESLIDLQ
ELGKYEQYIKWPWYIWLGFIAGLIAIVMVTIMLCCMTSCCSCLKGCCSCGSCCKFDEDDSEPVLKGVKLHYT
>P.2 (Based on GISAID ID: EPI_ISL_717936),
SEQ ID NO: 34
MFVFLVLLPLVSSQCVNLTTRTQLPPAYTNSFTRGVYYPDKVFRSSVLHSTQDLFLPFFSNVTWFHAIHVSGINGTKRED
NPVLPFNDGVYFASTEKSNIIRGWIFGTTLDSKTQSLLIVNNAINVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNERV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGSTPC
NGVKGFNCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSPGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSPIEDLLFNKVTLADAGFIKQYGDCLGDIAARDLICAQKFNGLTVLPPLLTDEMIAQYTSALLAG
TITSGWTFGAGPALQIPFPMQMAYRENGIGVTQNVLYENQKLIANQFNSAIGKIQDSLSSTPSALGKLQDVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASFVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
>A.23.1 (Based on GISAID ID: EPI_ISL_1469353),
SEQ ID NO: 35
MFVFLVLLPLVSSQCVNLTTRTQLPPAYTNSFTRGVYYPDKVFRSSVLHSTQDLFLPFFSNVTWFHAIHVSGTNGTKRED
NPVLPFNDGVYFASTEKSNIIRGWIFGTTLDSKTQSLLIVNNATNVVIKVCEFQFCNDPFLGVYYHKNNKSWMESELRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNERV
QPTESIVRFPNITNLCPFGEVENATRFASVYAWNRKRISNCVADYSFLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYHGVNCTEVPVAIHADQLTPTWRVYSTGS
NVFQTRAGCLIGAEHVNNSYECDIPIGAGICASYQTQTNSRGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSPIEDLLFNKVTLADAGFIKQYGDCLGDIAARDLICAQKFNGLTVLPPLLTDEMIAQYTSALLAG
TITSGWTFGAGPALQIPFPMQMAYRENGIGVTQNVLYENQKLIANQFNSAIGKIQDSLSSTPSALGKLQDVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
>B.1.526.2 (Based on GISAID ID: EPI_ISL_823886),
SEQ ID NO: 36
MFVFFVLLPLVSSQCVNLTTRTQLPPAYTNSFTRGVYYPDKVFRSSVLHSTQDLFLPFFSNVTWFHAIHVSGTNGTKRFD
NPVLPFNDGVYFASIEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGGSSSGWTAGAAAYYVGYLQPRTFLLKYNENGTITDAVDCALDPLSETKCTLKSFTVEKGIYQTSNFRV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGNTPC
NGVEGENCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSPGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSPIEDLLFNKVTLADAGFIKQYGDCLGDIAARDLICAQKFNGLTVLPPLLTDEMIAQYTSALLAG
TITSGWTFGAGPALQIPFPMQMAYRENGIGVTQNVLYENQKLIANQFNSAIGKIQDSLSSTPSALGKLQDVVNQNARALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
>P.3 (Based on GISAID ID: EPI_ISL_1073934),
SEQ ID NO: 37
MFVFLVLLPLVSSQCVNLTTRTQLPPAYTNSFTRGVYYPDKVFRSSVLHSTQDLFLPFFSNVTWFHAIHVSGTNGTKRED
NPVLPFNDGVYFASTEKSNIIRGWIFGTTLDSKTQSLLIVNNATNVVIKVCEFQFCNDPFYYHKNNKSWMESEFRVYSSA
NNCTFEYVSQPFLMDLEGKQGNFKNLREFVFKNIDGYFKIYSKHTPINLVRDLPQGFSALEPLVDLPIGINITRFQTLLH
RSYLTPGDSSSGWTAGAAACYVGYLQPRTFLLKYNENGTITDAVDCALDPLSETKCTLKSFTVEKGIYQTSNERVQPTES
IVRFPNITNLCPFGEVENATRFASVYAWNRKRISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYRLFRKSNLKPFERDISTEIYQAGSTPCNGVKG
FNCYFPLQSYGFQPTYGVGYQPYRVVVLSFELLHAPATVCGPKKSTNLVKNKCVNFNFNGLTGTGVLTESNKKELPFQQF
GRDIADTTDAVRDPQTLEILDITPCSFGGVSVITPGTNTSNQVAVLYQGVNCTEVPVAIHADQLTPTWRVYSTGSNVFQT
RAGCLIGAEHVNNSYECDIPIGAGICASYQTQTNSHGSASSVASQSIIAYTMSLGAENSVAYSNNSIAIPTNFTISVTTE
ILPVSMTKTSVDCTMYICGDSTECSNLLLQYGSFCTQLNRALTGIAVEQDKNTQEVFAQVKQIYKTPPIKDFGGENFSQI
LPDPSKPSKRSPIEDLLFNKVTLADAGFIKQYGDCLGDIAARDLICAQKFNGLTVLPPLLTDEMIAQYTSALLAGTITSG
WTFGAGPALQIPFPMQMAYRENGIGVTQNVLYENQKLIANQFNSAIGKIQDSLSSTPSALGKLQDVVNQNAQALNTLVKQ
LSSNFGAISSVLNDILSRLDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPRKGVFVSNGTYWFVTQRNFYEPQIITTDNTFVSGN
CDVVIGIVNNTVYDPLQPELDSFKEELDKYFKNHTSPDVDLGDISGINASFVNIQKEIDRLNEVAKNLNESLIDLQELGK
YEQYIKWPWYIWLGFIAGLIAIVMVTIMLCCMTSCCSCLKGCCSCGSCCKFDEDDSEPVLKGVKLHYT
>A.27 (Based on GISAID ID: EPI_ISL_985238),
SEQ ID NO: 38
MFVFLVLLPLVSSQCVNFTTRTQLPPAYTNSFTRGVYYPDKVFRSSVLHSTQDLFLPFFSNVTWFHAIHVSGTNGTKRFD
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNFRV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYRYRLFRKSNLKPFERDISTEIYQAGSTPC
NGVEGENCYFPLQSYGFQPTYGVGYQPYRVVVLSFELLHAPATVCGPKKSTNLVKNKCVNFNFNGLTGTGVLTESNKKFL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGVEYVNNSYECDIPIGAGICASYQTQTNSPGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKYFGGF
NFSQILPDPSKPSKRSPIEDLLFNKVTLADAGFIKQYGDCLGDIAARDLICAQKFNGLTVLPPLLTDEMIAQYTSALLAG
TITSGWTFGAGPALQIPFPMQMAYRENGIGVTQNVLYENQKLIANQFNSAIGKIQDSLSSTPSALGKLQDVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLVFIAGLIAIVMVTIMLCCMTSCCSCLKGCCSCGSCCKFDEDDSEPVLKGVKLHYT
>B.1.517,
SEQ ID NO: 39
MFVFLVLLPLVSSQCVNLTTRTQLPPAYTNSFTRGVYYPDKVFRSSVLHSTQDLFLPFFSNVTWFHAIHVSGTNGTKRED
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNFRV
QPTESIVRFPNITNLCPFGEVFNATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGSTPC
NGVEGENCYFPLQSYGFQPTTGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQDVNCTEVPVAIHADQLTPTWRVYSTGS
NVFQTRAGCLIGAEHVNNSYECDIPIGAGICASYQTQTNSPGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSPIEDLLFNKVTLADAGFIKQYGDCLGDIAARDLICAQKFNGLTVLPPLLTDEMIAQYTSALLAG
TITSGWTFGAGPALQIPFPMQMAYRFNGIGVTQNVLYENQKLIANQFNSAIGKIQDSLSSTPSALGKLQDVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
>Original B.1.526,
SEQ ID NO: 40
MFVFFVLLPLVSSQCVNLTTRTQLPPAYTNSFTRGVYYPDKVFRSSVLHSTQDLFLPFFSNVTWFHAIHVSGTNGTKRFD
NPVLPFNDGVYFASIEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGGSSSGWTAGAAAYYVGYLQPRTFLLKYNENGTITDAVDCALDPLSETKCTLKSFTVEKGIYQTSNFRV
QPTESIVRFPNITNLCPFGEVFNATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSPGSASSVASQSIIAYTMSLGVENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSPIEDLLFNKVTLADAGFIKQYGDCLGDIAARDLICAQKFNGLTVLPPLLTDEMIAQYTSALLAG
TITSGWTFGAGPALQIPFPMQMAYRENGIGVTQNVLYENQKLIANQFNSAIGKIQDSLSSTPSALGKLQDVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
>B.1.234,
SEQ ID NO: 41
MFVFLVLLPLVSSQCVNLTTRTQLPPAYTNSFTRGVYYPDKVFRSSVLHSTQDLFLPFFSNVTWFHAIHVSGTNGTKRED
NPVLPFNDGVYFASTEKSNIIRGWIFGTTLDSKTQSLLIVNNATNVVIKVCEFQFCNDPFLSVYYHKNNKSWMESEFRVY
SSANNCTFEYVSQPFLMDLVGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNERV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTHINSPGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSPIEDLLFNKVTLADAGFIKQYGDCLGDIAARDLICAQKFNGLTVLPPLLTDEMIAQYTSALLAG
TITSGWTFGAGPALQIPFPMQMAYRFNGIGVTQNVLYENQKLIANQFNSAIGKIQDSLSSTPSALGKLQDVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
>B.1.1.318,
SEQ ID NO: 42
MFVFLVLLPLVSSQCVNLTTRTQLPPAYTNSFTRGVYYPDKVFRSSVLHSTQDLFLPFFSNVTWFHAIHVSGTNGTKRFD
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNERV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGSTPC
NGVKGFNCYFPLQPYGFQPTYGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSHGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQTYKTPPIKDFGGF
NFSQILPDPSKPSKRSPIEDLLFNKVTLADAGFIKQYGDCLGDIAARDLICAQKFNGLTVLPPLLTDEMIAQYTSALLAG
TITSGWTFGAGPALQIPFPMQMAYRENGIGVTQNVLYENQKLIANQFNSAIGKIQDSLSSTPSALGKLQDVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYKPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
>B.1.616,
SEQ ID NO: 43
MFVFLVLLPLVSSQCVNLTTRTQLPPAYTNSFTRGVYYPDKVFRSSVLHSTQDLFLPFFSNVTWFDAIHVSGTNGTKRED
NPVLPFNDGVYFASTEKSNIIRGWIFGTTLDSKTQSLLIVNNATNVVIKVCEFQFCNDPFLVVYHKNNKSWMESEFRVYS
SANNCTFEYVSQPFLMDLEGKQGNFKNLREFVFKNIDGYFKIYSKHTPINLVRGLPQGFSALEPLVDLPIGINITRFQTL
LALHRSYLTPGDSSSGWTAGAAAYYVGYLQPRTFLLKYNENGTITDAVDCALDPLSETKCTLKSFTVEKGIYQTSNERVQ
PTESIVRFPNITNLCPFGEVENATRFASVYAWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNYLYRLFRKSNLKPFERDISTEIYQAGSTPCN
GAEGFNCYFPLQSYGFQPTNGVGYQPYRVVVLSFELLHAPATVCGPKKSTNLVKNKCVNFNFNGLTGTGVLTESNKKFLP
FQQFGRDIADTTDAVRDPQTLEILDITPCSFGGVSVITPGTNTSNQVAVLYQDVNCTEVPVAIHADQLTPTWRVYSTGSN
VFQTRAGCLIGAEYVNNSYECDIPIGASICASYQTQTNSPGSASSVASQSIIAYTMSLGAENSVAYSNNSIAIPTNFTIS
VTTEILPVSMTKTSVDCTMYICGDSTECSNLLLQYGSFCTQLNRALTGIAVEQDKNTQEVFAQVKQIYKTPPIKDFGGEN
FSQILPDPSKPSKRSPIEDLLFNKVTLADAGFIKQYGDCLGDIAARDLICAQKENGLTVLPPLLTDEMIAQYTSALLAGT
ITSGWTFGAGPALQIPFPMQMAYRENGIGVTQNVLYENQKLIANQFNSAIGKIQDSLSSTPSALGKLRDVVNQNAQALNT
LVKQLSSNFGAISSVLNDILSRLDPPEAEVQIDRLITGRLQSLQTYVTQQLIRAAEIRASANLAATKMSECVLGQSKRVD
FCGKGYHLMSFPQSAPHGVVFLHVTYVPAQEKNFTTAPAICHDGKDHFPREGVFVSNGTHWFVTQRNFYEPQIITTDNTF
VSGNCDVVIGIVNNTVYDPLQPELDSFKEELDKYFKNHTSPDVDLGDISGINASVVNIQKEIDRLNEVAKNLNESLIDLQ
ELGKYEQYIKWPWYIWLGFIAGLIAIVMVTIMLCCMTSCCSCLKGCCSCGSCCKFDEDDSEPVLKGVKLHYT
>B.1.575,
SEQ ID NO: 44
MFVFLVLLPLVSSQCVNLTTRTQLPPAYTNSFTRGVYYPDKVFRSSVLHSTQDLFLPFFSNVTWFHAIHVSGTNGTKRED
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTELLKYNENGTITDAVDCALDPLSETKCTLKSFTVEKGIYQTSNERV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGSTPC
NGVEGENCYFPLQPYGFQPTNGVGYQPYRVVVLSFELLHAPATVCGPKKSTNLVKNKCVNFNFNGLTGTGVLTESNKKFL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSHGSASSVASQSIIAYTMSLGAENSVAYSNNSIAIPINFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSPIEDLLFNKVTLADAGFIKQYGDCLGDIAARDLICAQKFNGLTVLPPLLTDEMIAQYTSALLAG
TITSGWTFGAGPALQIPFPMQMAYRENGIGVTQNVLYENQKLIANQFNSAIGKIQDSLSSTPSALGKLQDVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
>B.1.621,
SEQ ID NO: 16
MFVFLVLLPLVSSQCVNLTTRTQLPPAYTNSFTRGVYYPDKVFRSSVLHSTQDLFLPFFSNVTWFHAIHVSGTNGTKRED
NPVLPFNDGVYFASIEKSNIIRGWIFGTTLDSKTQSLLIVNNATNVVIKVCEFQFCNDPFLGTSNHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNERV
QPTESIVRFPNITNLCPFGEVENATKFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGSTPC
NGVKGFNCYFPLQSYGFQPTYGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSHGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDEGGF
NFSQILPDPSKPSKRSPIEDLLFNKVTLADAGFIKQYGDCLGDIAARDLICAQKENGLTVLPPLLTDEMIAQYTSALLAG
TITSGWTFGAGPALQIPFPMQMAYRENGIGVTQNVLYENQKLIANQFNSAIGKIQDSLSSTPSALGKLQNVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT

In some aspects, the recombinant SARS-CoV-2 S ectodomain included in the fusion protein comprises F817P, A892P, A899P, A942P, K986P, and V987P substitutions (numbering with reference to SEQ ID NO: 1) and an amino acid sequence at least 90% (such as at least 95%, at least 96%, at least 97%, at least 98%, or at least 99%) identical to any one of residues 14-1206 of SEQ ID NOs: 19 and 20; residues 14-1205 of SEQ ID NOs: 21-23, and 30; residues 14-1208 of SEQ ID NOs: 16, 24-25, 28-29, 31-32, 34-36, 38-42, and 44; residues 14-1201 of SEQ ID NO: 26; residues 14-1200 of SEQ ID NO: 27; residues 14-1203 of SEQ ID NO: 37; or residues 14-1207 of SEQ ID NOs: 33 and 43.

In some aspects, the recombinant SARS-CoV-2 S ectodomain included in the fusion protein comprises F817P, A892P, A899P, A942P, K986P, V987P, and a RRAR (682-685)-to-GSAS substitution (numbering with reference to SEQ ID NO: 1) and an amino acid sequence at least 90% (such as at least 95%, at least 96%, at least 97%, at least 98%, or at least 99%) identical to any one of residues 14-1206 of SEQ ID NOs: 19 and 20, residues 14-1205 of SEQ ID NOs: 21-23 and 30, residues 14-1208 of SEQ ID NOs: 16, 24-25, 28-29, 31-32, 34-36, 38-42, and 44, residues 14-1201 of SEQ ID NO: 26, residues 14-1200 of SEQ ID NO: 27, residues 14-1203 of SEQ ID NO: 37, or residues 14-1207 of SEQ ID NOs: 33 and 43.

In some aspects, the recombinant SARS-CoV-2 S ectodomain included in the fusion protein comprises or consists of any one of residues 14-1206 of SEQ ID NOs: 19 and 20, residues 14-1205 of SEQ ID NOs: 21-23 and 30, residues 14-1208 of SEQ ID NOs: 16, 24-25, 28-29, 31-32, 34-36, 38-42, and 44, residues 14-1201 of SEQ ID NO: 26, residues 14-1200 of SEQ ID NO: 27, residues 14-1203 of SEQ ID NO: 37, or residues 14-1207 of SEQ ID NOs: 33 and 43.

The recombinant SARS-CoV-2 S ectodomain included in the fusion protein is fused to HBsAg via a peptide linker. Any suitable peptide linker may be used that allows for folding and trimerization of the SARS-CoV-2 S ectodomain on the surface of the HBsAg nanoparticle. In some aspects, the peptide linker (such as a glycine-serine linker) is from 12-39 amino acids in length, such as 12-32, 16-32, 28-36, 28-32, 12-36, or 20-29, amino acids in length. In some aspects, the peptide linker (such as a glycine-serine linker) is any one of 12, 13, 14, 15, 16, 17, 18, 19, 20, 21, 22, 23, 24, 25, 26, 27, 28, 29, 30, 31, 32, 33, 34, 35, 36, 37, 38, or 39 amino acids in length. In some aspects, the peptide linker (such as a glycine-serine linker) is 12 amino acids in length. In some aspects, the peptide linker (such as a glycine-serine linker) is 16 amino acids in length. In some aspects, the peptide linker (such as a glycine-serine linker) is 24 amino acids in length. In some aspects, the peptide linker (such as a glycine-serine linker) is 32 amino acids in length. In some aspects, the peptide linker is composed of a repeat of GS di-peptides. In some aspects, the peptide linker has a sequence set forth as one of the 6-GS, 8-GS, 10-GS, 12-GS, 16-GS linkers:

(6-GS):
(SEQ ID NO: 4)
GSGSGSGSGSGS
(8-GS):
(SEQ ID NO: 5)
GSGSGSGSGSGSGSGS
(12-GS):
(SEQ ID NO: 6)
GSGSGSGSGSGSGSGSGSGSGSGS
(16-GS):
(SEQ ID NO: 7)
GSGSGSGSGSGSGSGSGSGSGSGSGSGSGSGS

The recombinant SARS-CoV-2 S ectodomain included in the fusion protein is fused to the HBsAg protein via the peptide linker. In several aspects, the HBsAg protein comprises or consists of a HBsAg S protein, a HBsAg M protein, or a HBsAg L protein. Exemplary HBsAg protein sequences are provided below:

>SEQ ID NO: 9:
MENTTSGFLGPLLVLQAGFFLLTRILTIPQSLDSWWTSLNFLGGAPTCPGQNSRSPISNHSPTSCPPICPGY
RWMCLRRFIIFLFILLLCLIFLLVLLDYQGMLPVCPLLPGTSTTSTGPCKTCTNPANGTSMFPSCCCTKPSD
GNCTCIPIPSSWAFARFLWEWASVRESWLSELVPFVQWFVGLSPTVWLSVMWMMWYWGPSLYNILSPFLPLL
PIFFCLWVYI
>adw (accession AY066028.1), SEQ ID NO: 45
MENTTSGFLGPLLVLQAGFFLLTRILTIPQSLDSWWTSLNFLGGAPTCPGQNSQSPISNHSPTSCPPICPGY
RWMCLRRFIIFLFILLLCLIFLLVLLDYQGMLPVCPLLPGTSTTSTGPCKTCTIPAQGTSMFPSCCCTKPSD
GNCTCIPIPSSWAFAKFLWEWASVRESWLSLLVPFVQWFVGLSPTVWLSVIWMMWYWGPRLYNILSPFLPLL
PIFFCLWVYI
>ayw (accession YP_009173871), SEQ ID NO: 46
MENITSGFLGPLLVLQAGFFLLTRILTIPQSLDSWWTSLNFLGGTTVCLGQNSQSPTSNHSPTSCPPTCPGY
RWMCLRRFIIFLFILLLCLIFLLVLLDYQGMLPVCPLIPGSSTTSTGPCRTCMTTAQGTSMYPSCCCTKPSD
GNCTCIPIPSSWAFGKELWEWASARESWLSLLVPFVQWFVGLSPTVWLSVIWMMWYWGPSLYSILSPFLPLL
PIFFCLWVYI
>ayr (accession AY738845.1), SEQ ID NO: 47
MESTTSGFLGPLLVLQAGFFLLTRILTIPQSLDSWWTSLNFLGGAPTCPGQNSQSPISNHSPTSCPPICPGY
RWMCLRRFIIFLFILLLCLIFLLVLLDYQGMLAVCPLLPGTSTTSTGPCRTCTIPAQGTSMFPSCCCTKPSD
GNCTCIPIPSSWAFARFLWEWASVRESWLSSLVPFVQWFAGLSPTVWLSVIWMMWYWGPSLYNILSPFLPLL
PIFFCLWVYI
>adr M (accession ABS18962.1), SEQ ID NO: 48
MQWNSSTFHQALLDPRVRGLYFPAGGSSSGTVNPVPTTASPISSIFSRTGDPAPNMESTTSGFLGPLLALQA
GFFLLTRILTIPQSLDSWWTSLNFLGGAPTCPGQNLQSPTSNHSPTSCPPICPGYRWMCLRRFIIFLFILLL
CLIFLLVLLDYQGMLPVCPLLPGTSTTSTGPCKTCTIPAQGTSMFPSCCCTKPSDGNCTCIPIPSSWAFARE
LWEWASVRFSWLSLLVPFVQWFVGLSPTVWLSVIWMMWYWGPSLYNILNPFLPLLPIFFCLWVYI
>adw M (accession ALG05526.1), SEQ ID NO: 49
MQWNSTTFHQTLQDPRVRALYFPAGGSSSGTLSPAQNTVSTISSILSKTGDPVPNMENIASGLLGPLLVLQA
GFFLLTKILTIPQSLDSWWTSLNFLGGTPVCLGQNSQSQISSHSPTCCPPICPGYRWMCLRRFIIFLCILLL
CLIFLLVLLDYQGMLPVCPLIPGSSTTSTGPCKTCTTPAQGTSMFPSCCCTKPTDGNCTCIPIPSSWAFAKY
LWEWASVRFSWLSLLVPFVQWFVGLSPTVWLSVIWMMWYWGPSLYNILSPFMPLLPIFFCLWVYI
>adw M (accession ADG03429.1), SEQ ID NO: 50
MQWNSTQFHQALLDPRVRGLYFPAGGSSSETQNPAPTIASLTSSIFSKTGDPAMNMENITSGLLGPLLVLQA
VCFLLTKILTIPQSLDSWWTSLNFLGVPPGCPGQNSQSPISNHLPTSCPPTCPGYRWMCLRRFIIFLFILLL
CLIFLLVLLDYQGMLPVCPLLPGSTTTSTGPCKTCTTLAQGTSMFPSCCCIKPSDGNCTCIPIPSSWAFGKY
LWEWASARFSWLSLLVQFVQWCVGLSPTVWLLVIWMIWYWGPNLCSILSPFIPLLPIFCYLWASI
>ayw M (accession AGL51218.1), SEQ ID NO: 51
MQWNSTTFHQTLQDPRVRALYFPAGGSSSGTVSPAQNTVSAISSILSKTGDPVPNMENIASGLLGPLLVLQA
GFFLLTKILTIPQSLDSWWTSLNFLGGTPVCLGQNSQSQTSSHSPTCCPPICPGYRWMCLRRFIIFLCILLL
CLIFLLVLLDYQGMLPVCPLIPGSSTTSTGPCRTCTTPAQGTSMFPSCCCTKPTDGNCTCIPIPSSWAFAKY
LWEWASVRFSWLSLLVPFVQWFVGLSPTVWLSVIWMMWEWGPSLYNILSPFMPLLPIFFCLWVYI
>ayr M (accession ADC29599.1), SEQ ID NO: 52
MQWNSTTFHQALLDPRVRGLYFPAGGSSSGTVNPVPTTASPISSILSKTGDPAPNMENITSGLLGPLLVLQA
GFFLLTKILTMPQSLDSWWISHNFLGGTPVCLGQNSQSQISSHSPTCCPPICPGYRWMCLRRFIIFLFILLL
CLIFLLVLLDYQGMLPVCPLLPGTSTTSTGTCRTCTIPAQGTSMFPSCCCTKPSAGNCTCIPIPSSWAFARE
LWEWASVRFSWLSLLVPFVQWFVGLSPTVWLSVIWMMWYWGPSLYNILSPFLPLLPIFFCLWVYI
>adr L (accession P03140), SEQ ID NO: 53
MGGWSSKPRQGMGTNLSVPNPLGFFPDHQLDPAFGANSNNPDWDENPNKDQWPEANQVGAGAFGPGFTPPHG
GLLGWSPQAQGILTTVPAAPPPASTNRQSGRQPTPISPPLRDSHPQAMQWNSTTFHQALLDPRVRGLYFPAG
GSSSGTVNPVPTTASPISSIFSRTGDPAPNMENTTSGFLGPLLVLQAGFFLLTRILTIPQSLDSWWTSLNFL
GGAPTCPGQNSQSPTSNHSPTSCPPICPGYRWMCLRRFIIFLFILLLCLIFLLVLLDYQGMLPVCPLLPGTS
TTSTGPCKTCTIPAQGTSMFPSCCCTKPSDGNCTCIPIPSSWAFARFLWEWASVRESWLSLLVPFVQWEVGL
SPTVWLSVIWMMWYWGPSLYNILSPFLPLLPIFFCLWVYI
>adw L (accession Q02317), SEQ ID NO: 54
MGGWSSKPRKGMGTNLSVPNPLGFFPDHQLDPAFGANSNNPDWDENPIKDHWPQANQVGVGAFGPGFTPPHG
GVLGWSPQAQGILATVPAMPPPASTNRQSGRQPTPISPPLRDSHPQAMQWNSTAFHQALQDPRVRGLYFPAG
GSSSGTLNPVPTIASHISSISSRIGDPAPNMENITSGELGPLLVLQAGFFLLTRILTIPQSLDSWWTSLNEL
GGAPVCLGQNSQSPTSNHSPTSCPPICPGYRWMCLRRFIIFLFILLLCLIFLLVLLDYQGMLPVCPLIPGST
TTSTGPCKTCTTPAQGNSMFPSCCCTKPTDGNCTCIPIPSSWAFAKYLWEWASVRESWLSLLVPFVQWEVGL
SPTVWLSAIWMMWYWGPSLYNILSPFIPLLPIFFCLWVYI
>ayw L (accession Q998L9), SEQ ID NO: 55
MGGWSSKHRKGMGTNLSVPNPLGFFPDHQLDPAFGANSNNPDWDENPNKDHWPEANQVGAGAFGPGFTPPHG
GFLGWSPQAQGILITVPAAPPPASTNRQSGRQPTPISPPLRDTHPQAMQWNSTAFHQALQDPRVRGLYFPAG
GSSSGTVNPVPNTVSHISSIFTKTGDPASNMESTTSGFLGPLLVLQAGFFLLTRILTIPQSLDSWWTSLNEL
GGAPGCIGQNSQSQTSNHSPTSCPPTCPGYRWMCLRRFIIFLFILLLCLIFLLVLLDYQGMLPVCPLLPGST
TTSTGPCRICTITAQGISMFPSCCCTKPSDGNCTCIPIPSSWGFAKFLWEWASVRESWLSLLVPFVQWFAGL
SPTVWLSVIWMIWYWGPSLYNILSPELPLLPIFLCLWVYI
>ayr L (accession Q76R62), SEQ ID NO: 56
MGGWSSKPRQGMGINLSVPNPLGFFPDHQLDPAFGANSNNPDWDENPNKDHWPEANQVGAGAFGPGFTPPHG
GLLGWSPQAQGILITLPAAPPPASTNRQSGRQPTPISPPLRDSHPQAMQWNSTTFHQALLDPRVRGLYFPAG
GSSSGTVNPVPTTASPISSIFSRTGDPAPNMESTTSGFLGPLLVLQAGFFLLTRILTIPQSLDSWWTSLNFL
GGAPTCPGQNSQSPTSNHSPTSCPPTCPGYRWMCLRRFIIFLFILLLCLIFLLVLLDYQGMLPVCPLLPGTS
TTSTGPCRTCTIPAQGTSMFPSCCCTKPSDGNCTCIPIPSSWAFARFLWEWASVRFSWLSLLVPFVQWEVGL
SPTVWLSAIWMMWYWGPSLYNILSPFLPLLPIFFCLWVYI

In some aspects, the HBsAg in the fusion protein comprises an amino acid sequence at least 90% (such as at least 95%, at least 96%, at least 97%, at least 98%, or at least 99%) identical to any one of SEQ ID NOs: 9 or 45-56. In some aspects, the HBsAg in the fusion protein comprises or consists of the amino acid sequence set forth as any one of SEQ ID NOs: 9 or 45-56.

Exemplary SARS-CoV-2/HBsAg fusion protein sequences encoded by the nucleic acid molecules are provided herein, including those listed in Table 1:

TABLE 1
Exemplary SARS-CoV-2 S ectodomain - HBsAg fusion proteins

| SEQ ID | |
|---|---|
| NO | Sequence description |
| | S6P-8-HBsAg S sequences, with SARS-CoV-2 sequence |
| | based on: |
| 10 | S6P(1-1206)-8-HBsAgS(1-226) |
| 57 | B.1.617.2 (Based on GISAID ID: EPI_ISL_2020950) |
| 58 | B.1.617.2 (Based on CDC website on Aug. 31, 2021) |
| 59 | B.1.1.7 (Based on GISAID ID: EPI_ISL_629440) |
| 60 | B.1.1.7 (Based on CDC website on Aug. 31, 2021) |
| 61 | B.1.351 (Based on GISAID ID: EPI_ISL_1120737) |
| 62 | P.1 (Based on GISAID ID: EPI_ISL_833171) |
| 63 | B.1.617.1 (Based on GISAID ID: EPI_ISL_1384866) |
| 64 | C.37-1 (Based on GISAID ID: EPI_ISL_1138413) |
| 65 | C.37-2 (Based on GISAID ID: EPI_ISL_1138413) |
| 66 | R.1 (Based on GISAID ID: EPI_ISL_1123466) |
| 67 | B.1.1.519 (Based on GISAID ID: EPI_ISL_876555) |
| 68 | B.1.525 (Based on GISAID ID: EPI_ISL_760883) |
| 69 | B.1.526 (Based on GISAID ID: EPI_ISL_1080798) |
| 70 | B.1.427/429 (Based on GISAID ID: EPI_ISL_984563 |
| | (B.1.427), EPI_ISL_672374 (B.1.429)) |
| 71 | B.1.526.1 (Based on GISAID ID: EPI_ISL_896306) |
| 72 | P.2 (Based on GISAID ID: EPI_ISL_717936) |
| 73 | A.23.1 (Based on GISAID ID: EPI_ISL_1469353) |
| 74 | B.1.526.2 (Based on GISAID ID: EPI_ISL_823886) |
| 75 | P.3 (Based on GISAID ID: EPI_ISL_1073934) |
| 76 | A.27 (Based on GISAID ID: EPI_ISL_985238) |
| 77 | B.1.517 |
| 78 | Original B.1.526 |
| 79 | B.1.234 |
| 80 | B.1.1.318 |
| 81 | B.1.616 |
| 82 | B.1.575 |
| 17 | B.1.621 |
| | S6P-12-HBsAg S sequences, with SARS-CoV-2 sequence |
| | based on: |
| 11 | S6P(1-1206)-12-HBsAgS(1-226) |
| 83 | B.1.617.2 (Based on GISAID ID: EPI_ISL_2020950) |
| 84 | B.1.617.2 (Based on CDC website on Aug. 31, 2021) |
| 85 | B.1.1.7 (Based on GISAID ID: EPI_ISL_629440) |
| 86 | B.1.1.7 (Based on CDC website on Aug. 31, 2021) |
| 87 | B.1.351-1 (Based on GISAID ID: EPI_ISL_1120737) |
| 88 | P.1 (Based on GISAID ID: EPI_ISL_833171) |
| 89 | B.1.617.1 (Based on GISAID ID: EPI_ISL_1384866) |
| 90 | C.37-1 (Based on GISAID ID: EPI_ISL_1138413) |
| 91 | C.37-2 (Based on GISAID ID: EPI_ISL_1138413) |
| 92 | R.1 (Based on GISAID ID: EPI_ISL_1123466) |
| 93 | B.1.1.519 (Based on GISAID ID: EPI_ISL_876555) |
| 94 | B.1.525 (Based on GISAID ID: EPI_ISL_760883) |
| 95 | B.1.526 (Based on GISAID ID: EPI_ISL_1080798) |
| 96 | B.1.427/429 (Based on GISAID ID: EPI_ISL_984563 |
| | (B.1.427), EPI_ISL_672374 (B.1.429)) |
| 97 | B.1.526.1 (Based on GISAID ID: EPI_ISL_896306) |
| 98 | P.2 (Based on GISAID ID: EPI_ISL_717936) |
| 99 | A.23.1 (Based on GISAID ID: EPI_ISL_1469353) |
| 100 | B.1.526.2 (Based on GISAID ID: EPI_ISL_823886) |
| 101 | P.3 (Based on GISAID ID: EPI_ISL_1073934) |
| 102 | A.27 (Based on GISAID ID: EPI_ISL_985238) |
| 103 | B.1.517 |
| 104 | Original B.1.526 |
| 105 | B.1.234 |
| 106 | B.1.1.318 |
| 107 | B.1.616 |
| 108 | B.1.575 |
| 18 | B.1.621 |
| | S6P-16-HBsAg S sequences, with SARS-CoV-2 sequence |
| | based on: |
| 12 | S6P(1-1206)-16-HBsAgS(1-226) |
| 109 | B.1.617.2 (Based on GISAID ID: EPI_ISL_2020950) |
| 110 | B.1.617.2 (Based on CDC website on Aug. 31, 2021) |
| 111 | B.1.1.7 (Based on GISAID ID: EPI_ISL_629440) |
| 112 | B.1.1.7 (Based on CDC website on Aug. 31, 2021) |
| 113 | B.1.351-1 (Based on GISAID ID: EPI_ISL_1120737) |
| 114 | P.1 (Based on GISAID ID: EPI_ISL_833171) |
| 115 | B.1.617.1 (Based on GISAID ID: EPI_ISL_1384866) |
| 116 | C.37-1 (Based on GISAID ID: EPI_ISL_1138413) |
| 117 | C.37-2 (Based on GISAID ID: EPI_ISL_1138413) |
| 118 | R.1 (Based on GISAID ID: EPI_ISL_1123466) |
| 119 | B.1.1.519 (Based on GISAID ID: EPI_ISL_876555) |
| 120 | B.1.525 (Based on GISAID ID: EPI_ISL_760883) |
| 121 | B.1.526 (Based on GISAID ID: EPI_ISL_1080798) |
| 122 | B.1.427/429 (Based on GISAID ID: EPI_ISL_984563 |
| | (B.1.427), EPI_ISL_672374 (B.1.429)) |
| 123 | B.1.526.1 (Based on GISAID ID: EPI_ISL_896306) |
| 124 | P.2 (Based on GISAID ID: EPI_ISL_717936) |
| 125 | A.23.1 (Based on GISAID ID: EPI_ISL_1469353) |
| 126 | B.1.526.2 (Based on GISAID ID: EPI_ISL_823886) |
| 127 | P.3 (Based on GISAID ID: EPI_ISL_1073934) |
| 128 | A.27 (Based on GISAID ID: EPI_ISL_985238) |
| 129 | B.1.517 |
| 130 | Original B.1.526 |
| 131 | B.1.234 |
| 132 | B.1.1.318 |
| 133 | B.1.616 |
| 134 | B.1.575 |
| 315 | B.1.621 |
| | S6P-8-HBsAg M sequences, with SARS-CoV-2 sequence |
| | based on: |
| 135 | S6P |
| 136 | B.1.617.2 (Based on GISAID ID: EPI_ISL_2020950) |
| 137 | B.1.617.2 (Based on CDC website on Aug. 31, 2021) |
| 138 | B.1.1.7 (Based on GISAID ID: EPI_ISL_629440) |
| 139 | B.1.1.7 (Based on CDC website on Aug. 31, 2021) |
| 140 | B.1.351-1 (Based on GISAID ID: EPI_ISL_1120737) |
| 141 | P.1 (Based on GISAID ID: EPI_ISL_833171) |
| 142 | B.1.617.1 (Based on GISAID ID: EPI_ISL_1384866) |
| 143 | C.37-1 (Based on GISAID ID: EPI_ISL_1138413) |
| 144 | C.37-2 (Based on GISAID ID: EPI_ISL_1138413) |
| 145 | R.1 (Based on GISAID ID: EPI_ISL_1123466) |
| 146 | B.1.1.519 (Based on GISAID ID: EPI_ISL_876555) |
| 147 | B.1.525 (Based on GISAID ID: EPI_ISL_760883) |
| 148 | B.1.526 (Based on GISAID ID: EPI_ISL_1080798) |
| 149 | B.1.427/429 (Based on GISAID ID: EPI_ISL_984563 |
| | (B.1.427), EPI_ISL_672374 (B.1.429)) |
| 150 | B.1.526.1 (Based on GISAID ID: EPI_ISL_896306) |
| 151 | P.2 (Based on GISAID ID: EPI_ISL_717936) |
| 152 | A.23.1 (Based on GISAID ID: EPI_ISL_1469353) |
| 153 | B.1.526.2 (Based on GISAID ID: EPI_ISL_823886) |
| 154 | P.3 (Based on GISAID ID: EPI_ISL_1073934) |
| 155 | A.27 (Based on GISAID ID: EPI_ISL_985238) |
| 156 | B.1.517 |
| 157 | Original B.1.526 |
| 158 | B.1.234 |
| 159 | B.1.1.318 |
| 160 | B.1.616 |
| 161 | B.1.575 |
| 316 | B.1.621 |
| | S6P-12-HBsAg M sequences, with SARS-CoV-2 sequence |
| | based on: |
| 162 | S6P |
| 163 | B.1.617.2 (Based on GISAID ID: EPI_ISL_2020950) |
| 164 | B.1.617.2 (Based on CDC website on Aug. 31, 2021) |
| 165 | B.1.1.7 (Based on GISAID ID: EPI_ISL_629440) |
| 166 | B.1.1.7 (Based on CDC website on Aug. 31, 2021) |
| 167 | B.1.351-1 (Based on GISAID ID: EPI_ISL_1120737) |
| 168 | P.1 (Based on GISAID ID: EPI_ISL_833171) |
| 169 | B.1.617.1 (Based on GISAID ID: EPI_ISL_1384866) |
| 170 | C.37-1 (Based on GISAID ID: EPI_ISL_1138413) |
| 171 | C.37-2 (Based on GISAID ID: EPI_ISL_1138413) |
| 172 | R.1 (Based on GISAID ID: EPI_ISL_1123466) |
| 173 | B.1.1.519 (Based on GISAID ID: EPI_ISL_876555) |
| 174 | B.1.525 (Based on GISAID ID: EPI_ISL_760883) |
| 175 | B.1.526 (Based on GISAID ID: EPI_ISL_1080798) |
| 176 | B.1.427/429 (Based on GISAID ID: EPI_ISL_984563 |
| | (B.1.427), EPI_ISL_672374 (B.1.429)) |
| 177 | B.1.526.1 (Based on GISAID ID: EPI_ISL_896306) |
| 178 | P.2 (Based on GISAID ID: EPI_ISL_717936) |
| 179 | A.23.1 (Based on GISAID ID: EPI_ISL_1469353) |
| 180 | B.1.526.2 (Based on GISAID ID: EPI_ISL_823886) |
| 181 | P.3 (Based on GISAID ID: EPI_ISL_1073934) |
| 182 | A.27 (Based on GISAID ID: EPI_ISL_985238) |
| 183 | B.1.517 |
| 184 | Original B.1.526 |
| 185 | B.1.234 |
| 186 | B.1.1.318 |
| 187 | B.1.616 |
| 188 | B.1.575 |
| 317 | B.1.621 |
| | S6P-16-HBsAg M sequences, with SARS-CoV-2 sequence |
| | based on: |
| 189 | S6P |
| 190 | B.1.617.2 (Based on GISAID ID: EPI_ISL_2020950) |
| 191 | B.1.617.2 (Based on CDC website on Aug. 31, 2021) |
| 192 | B.1.1.7 (Based on GISAID ID: EPI_ISL_629440) |
| 193 | B.1.1.7 (Based on CDC website on Aug. 31, 2021) |
| 194 | B.1.351-1 (Based on GISAID ID: EPI_ISL_1120737) |
| 195 | P.1 (Based on GISAID ID: EPI_ISL_833171) |
| 196 | B.1.617.1 (Based on GISAID ID: EPI_ISL_1384866) |
| 197 | C.37-1 (Based on GISAID ID: EPI_ISL_1138413) |
| 198 | C.37-2 (Based on GISAID ID: EPI_ISL_1138413) |
| 199 | R.1 (Based on GISAID ID: EPI_ISL_1123466) |
| 200 | B.1.1.519 (Based on GISAID ID: EPI_ISL_876555) |
| 201 | B.1.525 (Based on GISAID ID: EPI_ISL_760883) |
| 202 | B.1.526 (Based on GISAID ID: EPI_ISL_1080798) |
| 203 | B.1.427/429 (Based on GISAID ID: EPI_ISL_984563 |
| | (B.1.427), EPI_ISL_672374 (B.1.429)) |
| 204 | B.1.526.1 (Based on GISAID ID: EPI_ISL_896306) |
| 205 | P.2 (Based on GISAID ID: EPI_ISL_717936) |
| 206 | A.23.1 (Based on GISAID ID: EPI_ISL_1469353) |
| 207 | B.1.526.2 (Based on GISAID ID: EPI_ISL_823886) |
| 208 | P.3 (Based on GISAID ID: EPI_ISL_1073934) |
| 209 | A.27 (Based on GISAID ID: EPI_ISL_985238) |
| 210 | B.1.517 |
| 211 | Original B.1.526 |
| 212 | B.1.234 |
| 213 | B.1.1.318 |
| 214 | B.1.616 |
| 215 | B.1.575 |
| 318 | B.1.621 |
| | S6P-8-HBsAg L sequences, with SARS-CoV-2 sequence |
| | based on: |
| 216 | S6P |
| 217 | B.1.617.2 (Based on GISAID ID: EPI_ISL_2020950) |
| 218 | B.1.617.2 (Based on CDC website on Aug. 31, 2021) |
| 219 | B.1.1.7 (Based on GISAID ID: EPI_ISL_629440) |
| 220 | B.1.1.7 (Based on CDC website on Aug. 31, 2021) |
| 221 | B.1.351-1 (Based on GISAID ID: EPI_ISL_1120737) |
| 222 | P.1 (Based on GISAID ID: EPI_ISL_833171) |
| 223 | B.1.617.1 (Based on GISAID ID: EPI_ISL_1384866) |
| 224 | C.37-1 (Based on GISAID ID: EPI_ISL_1138413) |
| 225 | C.37-2 (Based on GISAID ID: EPI_ISL_1138413) |
| 226 | R.1 (Based on GISAID ID: EPI_ISL_1123466) |
| 227 | B.1.1.519 (Based on GISAID ID: EPI_ISL_876555) |
| 228 | B.1.525 (Based on GISAID ID: EPI_ISL_760883) |
| 229 | B.1.526 (Based on GISAID ID: EPI_ISL_1080798) |
| 230 | B.1.427/429 (Based on GISAID ID: EPI_ISL_984563 |
| | (B.1.427), EPI_ISL_672374 (B.1.429)) |
| 231 | B.1.526.1 (Based on GISAID ID: EPI_ISL_896306) |
| 232 | P.2 (Based on GISAID ID: EPI_ISL_717936) |
| 233 | A.23.1 (Based on GISAID ID: EPI_ISL_1469353) |
| 234 | B.1.526.2 (Based on GISAID ID: EPI_ISL_823886) |
| 235 | P.3 (Based on GISAID ID: EPI_ISL_1073934) |
| 236 | A.27 (Based on GISAID ID: EPI_ISL_985238) |
| 237 | B.1.517 |
| 238 | Original B.1.526 |
| 239 | B.1.234 |
| 240 | B.1.1.318 |
| 241 | B.1.616 |
| 242 | B.1.575 |
| 319 | B.1.621 |
| | S6P-12-HBsAg L sequences, with SARS-CoV-2 sequence |
| | based on: |
| 243 | S6P |
| 244 | B.1.617.2 (Based on GISAID ID: EPI_ISL_2020950) |
| 245 | B.1.617.2 (Based on CDC website on Aug. 31, 2021) |
| 246 | B.1.1.7 (Based on GISAID ID: EPI_ISL_629440) |
| 247 | B.1.1.7 (Based on CDC website on Aug. 31, 2021) |
| 248 | B.1.351-1 (Based on GISAID ID: EPI_ISL_1120737) |
| 249 | P.1 (Based on GISAID ID: EPI_ISL_833171) |
| 250 | B.1.617.1 (Based on GISAID ID: EPI_ISL_1384866) |
| 251 | C.37-1 (Based on GISAID ID: EPI_ISL_1138413) |
| 252 | C.37-2 (Based on GISAID ID: EPI_ISL_1138413) |
| 253 | R.1 (Based on GISAID ID: EPI_ISL_1123466) |
| 254 | B.1.1.519 (Based on GISAID ID: EPI_ISL_876555) |
| 255 | B.1.525 (Based on GISAID ID: EPI_ISL_760883) |
| 256 | B.1.526 (Based on GISAID ID: EPI_ISL_1080798) |
| 257 | B.1.427/429 (Based on GISAID ID: EPI_ISL_984563 |
| | (B.1.427), EPI_ISL_672374 (B.1.429)) |
| 258 | B.1.526.1 (Based on GISAID ID: EPI_ISL_896306) |
| 259 | P.2 (Based on GISAID ID: EPI_ISL_717936) |
| 260 | A.23.1 (Based on GISAID ID: EPI_ISL_1469353) |
| 261 | B.1.526.2 (Based on GISAID ID: EPI_ISL_823886) |
| 262 | P.3 (Based on GISAID ID: EPI_ISL_1073934) |
| 263 | A.27 (Based on GISAID ID: EPI_ISL_985238) |
| 264 | B.1.517 |
| 265 | Original B.1.526 |
| 266 | B.1.234 |
| 267 | B.1.1.318 |
| 268 | B.1.616 |
| 269 | B.1.575 |
| 320 | B.1.621 |
| | S6P-16-HBsAg L sequences, with SARS-CoV-2 sequence |
| | based on: |
| 270 | S6P |
| 271 | B.1.617.2 (Based on GISAID ID: EPI_ISL_2020950) |
| 272 | B.1.617.2 (Based on CDC website on Aug. 31, 2021) |
| 273 | B.1.1.7 (Based on GISAID ID: EPI_ISL_629440) |
| 274 | B.1.1.7 (Based on CDC website on Aug. 31, 2021) |
| 275 | B.1.351-1 (Based on GISAID ID: EPI_ISL_1120737) |
| 276 | P.1 (Based on GISAID ID: EPI_ISL_833171) |
| 277 | B.1. 617.1 (Based on GISAID ID: EPI_ISL_1384866) |
| 278 | C.37-1 (Based on GISAID ID: EPI_ISL_1138413) |
| 279 | C.37-2 (Based on GISAID ID: EPI_ISL_1138413) |
| 280 | R.1 (Based on GISAID ID: EPI_ISL_1123466) |
| 281 | B.1.1.519 (Based on GISAID ID: EPI_ISL_876555) |
| 282 | B.1.525 (Based on GISAID ID: EPI_ISL_760883) |
| 283 | B.1.526 (Based on GISAID ID: EPI_ISL_1080798) |
| 284 | B.1.427/429 (Based on GISAID ID: EPI_ISL_984563 |
| | (B.1.427), EPI_ISL_672374 (B.1.429)) |
| 285 | B.1.526.1 (Based on GISAID ID: EPI_ISL_896306) |
| 286 | P.2 (Based on GISAID ID: EPI_ISL_717936) |
| 287 | A.23.1 (Based on GISAID ID: EPI_ISL_1469353) |
| 288 | B.1.526.2 (Based on GISAID ID: EPI_ISL_823886) |
| 289 | P.3 (Based on GISAID ID: EPI_ISL_1073934) |
| 290 | A.27 (Based on GISAID ID: EPI_ISL_985238) |
| 291 | B.1.517 |
| 292 | Original B.1.526 |
| 293 | B.1.234 |
| 294 | B.1.1.318 |
| 295 | B.1.616 |
| 296 | B.1.575 |
| 321 | B.1.621 |

The fusion protein sequences provided in Table 1 include a signal peptide (residues 1-13) that is removed from the protein as expressed in mammalian cells. In some aspects, the fusion protein encoded by the nucleic acid molecule comprises or consists of an amino acid sequence at least 90% (such as at least 95%, at least 96%, at least 97%, at least 98%, at least 99%) identical to residues 14 to the C-terminus of any one of the SARS-CoV-2-HBsAg fusion proteins provided in Table 1. For example, the fusion protein comprises or consists of an amino acid sequence at least 90% (such as at least 95%, at least 96%, at least 97%, at least 98%, at least 99%) identical to residues 14 to the C-terminus of any one of SEQ ID NOs: 10-12, 17-18, or 57-321, wherein the SARS-CoV-2 S ectodomain in the fusion protein includes F817P, A892P, A899P, A942P, K986P, V987P, and RRAR (682-685)-to-GSAS substitutions according to the reference SARS-CoV-2 S set forth as SEQ ID NO: 1.

In some aspects, the fusion protein encoded by the nucleic acid molecule comprises or consists of the amino acid sequence set forth as residues 14 to the C-terminus of any one of the SARS-CoV-1 HBsAg fusion proteins provided in Table 1, such as any one of SEQ ID NOs: 10-12, 17-18, or 57-503. When a nucleic acid molecule encoding the fusion protein is expressed in mammalian cells, the fusion protein self-assembles to form a HBsAg nanoparticle with SARS-CoV-2 S ectodomain trimers extending radially outward from an outer surface of the HBsAg nanoparticle.

Exemplary DNA sequences encoding fusion protein sequences as disclosed herein are provided as the following:

S6P-8-HBsAg
(SEQ ID NO: 13)
ATGTTCGTGTTTCTGGTGCTGCTGCCCCTGGTGAGCTCCCAGTGCGTGAACCTGACCACAAGGACCCAGCTG
CCCCCTGCCTATACCAATTCCTTTACAAGGGGCGTGTACTATCCAGACAAGGTGTTCCGCTCTAGCGTGCTG
CACTCTACACAGGATCTGTTCCTGCCCTTCTTTAGCAACGIGACCTGGTTTCACGCCATCCACGTGAGCGGC
ACCAATGGCACAAAGCGGTTTGACAATCCTGTGCTGCCATTCAACGATGGCGTGTACTTTGCCTCCACCGAG
AAGTCTAACATCATCAGAGGCTGGATCTTCGGCACCACACTGGACAGCAAGACACAGTCCCTGCTGATCGTG
AACAATGCCACCAACGTGGTCATCAAGGTGTGCGAGTTCCAGTTTTGCAATGATCCCTTTCTGGGCGTGTAC
TATCACAAGAACAATAAGICTTGGATGGAGAGCGAGTTCCGCGTGTATTCCICTGCCAACAATTGCACATTC
GAGTACGTGAGCCAGCCTTTTCTGATGGACCTGGAGGGCAAGCAGGGCAACTTCAAGAACCTGAGGGAGTTC
GTGTTTAAGAATATCGATGGCTATTICAAGATCTACTCTAAGCACACCCCAATCAACCTGGTGCGCGACCTG
CCACAGGGCTTTAGCGCCCTGGAGCCCCTGGTGGATCTGCCTATCGGCATCAACATCACCCGGTTCCAGACA
CTGCTGGCCCTGCACAGAAGCTACCTGACACCCGGCGACAGCTCCTCTGGATGGACCGCAGGAGCAGCAGCC
TACTATGTGGGCTATCTGCAGCCTCGGACCTTTCTGCTGAAGTACAACGAGAATGGCACCATCACAGACGCC
GIGGATTGTGCCCTGGACCCCCTGAGCGAGACCAAGTGCACACTGAAGTCCTTCACCGTGGAGAAGGGCATC
TATCAGACATCCAACTTCCGGGTGCAGCCAACCGAGTCTATCGTGCGCTTCCCAAATATCACAAACCTGTGC
CCCTTTGGCGAGGIGTTCAACGCCACCAGATTCGCCAGCGTGTACGCCTGGAATAGGAAGCGCATCTCTAAC
TGCGTGGCCGACTATAGCGTGCTGTACAACTCCGCCTCTTTCAGCACCTTTAAGTGTTATGGCGTGTCCCCC
ACAAAGCTGAATGATCTGTGCTTCACCAACGTGTACGCCGATTCTTTTGTGATCAGGGGCGACGAGGIGCGC
CAGATCGCACCAGGACAGACAGGCAAGATCGCCGACTACAATTATAAGCTGCCCGACGATTTCACCGGCTGC
GTGATCGCCTGGAACTCCAACAATCIGGATTCTAAAGTGGGCGGCAACTACAATTATCTGTACCGGCTGTTC
AGAAAGTCCAATCTGAAGCCTTTTGAGAGGGACATCTCTACAGAGATCTACCAGGCCGGCAGCACCCCATGT
AATGGCGTGGAGGGCTTTAACTGCTATTTCCCTCTGCAGAGCTACGGCTTTCAGCCAACCAACGGCGTGGGC
TATCAGCCCTACCGCGTGGIGGTGCTGAGCTTCGAGCTGCTGCACGCACCTGCAACCGTGTGCGGACCAAAG
AAGTCCACCAATCTGGTGAAGAACAAGTGCGTGAACTTCAACTTCAACGGACTGACCGGAACAGGCGIGCTG
ACCGAGTCCAACAAGAAGTTCCTGCCATTTCAGCAGTTCGGCCGGGACATCGCCGATACCACAGACGCCGTG
AGAGACCCTCAGACCCTGGAGATCCTGGATATCACACCATGTAGCTTCGGCGGCGTGTCCGTGATCACACCA
GGAACCAATACAAGCAACCAGGTGGCCGTGCTGTATCAGGACGTGAATTGCACCGAGGTGCCAGTGGCAATC
CACGCAGATCAGCTGACCCCTACATGGAGGGTGTACTCCACCGGCTCTAACGTGTTCCAGACAAGGGCAGGA
TGCCTGATCGGAGCAGAGCACGTGAACAATTCCTATGAGTGTGACATCCCAATCGGAGCAGGAATCTGCGCA
TCTTACCAGACCCAGACAAACAGCCCTGGCTCCGCCAGCTCCGTGGCATCCCAGTCTATCATCGCCTATACC
ATGAGCCTGGGCGCCGAGAATTCCGTGGCCTACTCTAACAATAGCATCGCCATCCCCACCAACTTTACAATC
TCCGTGACCACAGAGATCCTGCCCGTGAGCATGACCAAGACAAGCGTGGACTGTACAATGTATATCTGCGGC
GATTCCACCGAGTGTTCTAACCTGCTGCTGCAGTACGGCAGCTTCTGCACCCAGCTGAATAGGGCCCTGACA
GGAATCGCAGTGGAGCAGGACAAGAACACACAGGAGGTGTTTGCCCAGGTGAAGCAGATCTACAAGACCCCA
CCCATCAAGGACTTCGGCGGCTTCAACTTCAGCCAGATCCTGCCTGATCCATCCAAGCCATCTAAGCGGAGC
CCCATCGAGGACCTGCTGTTCAACAAGGTGACCCTGGCCGATGCCGGCTTTATCAAGCAGTATGGCGAITGT
CTGGGCGACATCGCCGCCAGAGACCTGATCTGCGCCCAGAAGTTTAATGGCCTGACCGTGCTGCCTCCACTG
CTGACAGATGAGAIGATCGCACAGTACACAAGCGCCCTGCTGGCAGGAACCATCACATCCGGATGGACCTTC
GGCGCAGGACCTGCCCTGCAGATCCCCTTCCCTATGCAGATGGCCTATCGGITCAACGGCATCGGCGTGACC
CAGAATGTGCTGTACGAGAACCAGAAGCTGATCGCCAATCAGITTAACTCCGCCATCGGCAAGATCCAGGAC
TCTCTGTCTAGCACACCAAGCGCCCTGGGCAAGCTGCAGGATGTGGTGAATCAGAACGCCCAGGCCCTGAAT
ACCCTGGTGAAGCAGCTGTCCTCTAACTTCGGCGCCATCAGCTCCGTGCTGAATGATATCCTGAGCCGGCTG
GACCCCCCTGAGGCAGAGGTGCAGATCGACCGGCTGATCACAGGCAGACTGCAGTCTCTGCAGACCTACGTG
ACACAGCAGCTGATCAGGGCAGCAGAGATCAGGGCAAGCGCCAATCTGGCAGCAACCAAGATGTCCGAGTGC
GTGCTGGGCCAGTCTAAGAGAGTGGACTTCTGCGGCAAGGGCTAICACCTGATGAGCTTTCCTCAGTCCGCC
CCACACGGAGTGGTGTTCCTGCACGTGACCTACGTGCCAGCCCAGGAGAAGAACTTCACCACAGCCCCCGCC
ATCTGTCACGATGGCAAGGCACACTTCCCAAGGGAGGGCGTGTTTGTGAGCAACGGCACCCACTGGTTCGTG
ACACAGAGAAACTTCTACGAGCCCCAGATCATCACCACAGACAATACATTCGTGTCCGGCAACTGCGACGTG
GICATCGGCATCGTGAACAATACCGTGTATGATCCCCTGCAGCCTGAGCTGGACAGCTICAAGGAGGAGCTG
GATAAGTACTTTAAGAATCACACCTCCCCTGACGTGGATCTGGGCGACATCTCCGGCATCAATGCCTCTGTG
GTGAACATCCAGAAGGAGATCGACAGGCTGAACGAGGTGGCCAAGAATCTGAACGAGTCTCTGATCGATCTG
CAGGAGCTGGGCAAGTACGGCAGCGGCTCCGGCTCTGGCAGCGGCTCCGGCTCTGGCAGCGGCTCCATGGAG
AATACCACATCCGGCTTTCTGGGACCTCTGCTGGTGCTGCAGGCAGGCTTCITTCTGCTGACCCGCATCCTG
ACAATCCCACAGTCCCTGGATTCTTGGTGGACAAGCCTGAACTTCCTGGGAGGAGCACCAACCTGTCCAGGA
CAGAATTCTAGGAGCCCCACCAGCAACCACTCCCCTACATCTTGTCCACCCATCTGCCCAGGATATAGGIGG
ATGTGCCTGCGGCGGTTCATCATCTTCCTGITTATCCTGCTGCTGTGCCTGATCTTCCTGCTGGTGCTGCTG
GACTACCAGGGCATGCTGCCCGTGTGCCCACTGCTGCCAGGCACCAGCACCACATCCACAGGACCTTGTAAG
ACCTGCACAAATCCAGCCAACGGCACCTCTATGTTTCCCAGCTGCTGTTGCACAAAGCCTTCCGATGGCAAT
TGTACCTGCATCCCAATCCCCTCTAGCTGGGCCTTTGCCCGGTTCCTGTGGGAGTGGGCCTCTGTGAGATTC
TCTTGGCTGAGCTTTCTGGTGCCTTTCGTGCAGTGGTTTGTGGGCCTGTCCCCAACCGTGTGGCTGTCTGTG
ATGTGGATGATGTGGTATTGGGGCCCCAGCCTGTACAACATCCTGTCCCCTTTCCTGCCACTGCTGCCCATC
TTCTTTTGCCTGTGGGTGTACATCTGA
S6P-12-HBsAg
(SEQ ID NO: 14)
ATGTTCGTGTTTCTGGTGCTGCTGCCCCTGGTGAGCTCCCAGTGCGTGAACCTGACCACAAGGACCCAGCTG
CCCCCTGCCTATACCAATAGCTTTACAAGGGGCGTGTACTATCCAGACAAGGTGTTCCGCTCTAGCGTGCTG
CACAGCACACAGGATCTGTTCCTGCCCTTCTTTTCCAACGTGACCTGGTTTCACGCCATCCACGTGTCCGGC
ACCAATGGCACAAAGCGGTTTGACAATCCTGTGCTGCCATTCAACGATGGCGTGTACTTTGCCTCTACCGAG
AAGAGCAACATCATCAGAGGCTGGATCTTCGGCACCACACTGGACTCCAAGACACAGTCTCTGCTGATCGTG
AACAATGCCACCAACGTGGTCATCAAGGTGTGCGAGTTCCAGTTTTGCAATGATCCCTTTCTGGGCGTGTAC
TATCACAAGAACAATAAGAGCTGGATGGAGTCCGAGTTCCGCGTGTATTCCTCTGCCAACAATTGCACATTC
GAGTACGTGAGCCAGCCTTTTCTGATGGACCTGGAGGGCAAGCAGGGCAACTTCAAGAACCTGAGGGAGTTC
GTGTTTAAGAATATCGATGGCTATTTCAAGATCTACAGCAAGCACACCCCAATCAACCTGGTGCGCGACCTG
CCACAGGGCTTTTCCGCCCTGGAGCCCCTGGTGGATCTGCCTATCGGCATCAACATCACCCGGTTCCAGACA
CTGCTGGCCCTGCACAGATCCTACCTGACACCCGGCGACAGCTCCTCTGGATGGACCGCAGGAGCAGCAGCC
TACTATGTGGGCTATCTGCAGCCTCGGACCTTTCTGCTGAAGTACAACGAGAATGGCACCATCACAGACGCC
GTGGATTGTGCCCTGGACCCCCTGTCCGAGACCAAGTGCACACTGAAGTCTTTCACCGTGGAGAAGGGCATC
TATCAGACATCTAACTTCCGGGTGCAGCCAACCGAGAGCATCGTGCGCTTCCCAAATATCACAAACCTGTGC
CCCTTTGGCGAGGIGTTCAACGCCACCAGATTCGCCAGCGTGTACGCCTGGAATAGGAAGCGCATCAGCAAC
TGCGTGGCCGACTATTCCGTGCTGTACAACTCCGCCTCTTTCAGCACCTTTAAGTGTTATGGCGTGTCTCCC
ACAAAGCTGAATGATCTGTGCTTCACCAACGTGTACGCCGATAGCTTTGTGATCAGGGGCGACGAGGTGCGC
CAGATCGCACCAGGACAGACAGGCAAGATCGCCGACTACAATTATAAGCTGCCCGACGATTTCACCGGCTGC
GTGATCGCCTGGAACTCTAACAATCTGGATAGCAAAGTGGGCGGCAACTACAATTATCTGTACCGGCTGTTC
AGAAAGAGCAATCTGAAGCCTTTTGAGAGGGACATCAGCACAGAGATCTACCAGGCCGGCTCCACCCCATGT
AATGGCGTGGAGGGCTTTAACTGCTATTTCCCTCTGCAGTCCTACGGCITTCAGCCAACCAACGGCGTGGGC
TATCAGCCCTACCGCGTGGTGGTGCTGTCCTTCGAGCTGCTGCACGCACCTGCAACCGTGTGCGGACCAAAG
AAGTCTACCAATCTGGTGAAGAACAAGTGCGTGAACTTCAACTTCAACGGACTGACCGGAACAGGCGTGCTG
ACCGAGAGCAACAAGAAGTTCCTGCCATTTCAGCAGTTCGGCCGGGACATCGCCGATACCACAGACGCCGTG
AGAGACCCTCAGACCCTGGAGATCCTGGATATCACACCATGTTCCTTCGGCGGCGTGTCTGTGATCACACCC
GGCACCAATACATCCAACCAGGTGGCCGTGCTGTATCAGGACGTGAATTGCACCGAGGTGCCAGTGGCAATC
CACGCAGATCAGCIGACCCCTACATGGAGGGTGTACTCTACCGGCAGCAACGTGTTCCAGACAAGGGCAGGA
TGCCTGATCGGAGCAGAGCACGTGAACAATTCTTATGAGTGTGACATCCCAATCGGAGCAGGAATCTGCGCA
AGCTACCAGACCCAGACAAACTCCCCTGGCTCTGCCAGCTCCGTGGCATCCCAGTCTATCATCGCCTATACC
ATGTCCCTGGGCGCCGAGAATTCTGTGGCCTACAGCAACAATTCCATCGCCATCCCCACCAACTTTACAATC
AGCGTGACCACAGAGATCCTGCCTGTGAGCATGACCAAGACATCCGTGGACTGTACAATGTATATCTGCGGC
GATTCTACCGAGTGTAGCAACCTGCTGCTGCAGTACGGCTCCTTCTGCACCCAGCTGAATAGGGCCCTGACA
GGAATCGCAGTGGAGCAGGACAAGAACACACAGGAGGTGTTTGCCCAGGTGAAGCAGATCTACAAGACCCCA
CCCATCAAGGACTICGGCGGCTTCAACTTCAGCCAGATCCTGCCTGATCCATCTAAGCCAAGCAAGCGGTCC
CCCATCGAGGACCTGCTGTTCAACAAGGTGACCCTGGCCGATGCCGGCTTTATCAAGCAGTATGGCGATTGT
CTGGGCGACATCGCCGCCAGAGACCTGATCTGCGCCCAGAAGTTTAATGGCCTGACCGTGCTGCCTCCACTG
CTGACAGATGAGAIGATCGCACAGTACACATCCGCCCTGCTGGCAGGCACCATCACATCTGGATGGACCTTC
GGCGCAGGACCTGCCCTGCAGATCCCCTTCCCTATGCAGATGGCCTATCGGTTCAACGGCATCGGCGTGACC
CAGAATGTGCTGTACGAGAACCAGAAGCTGATCGCCAATCAGTTTAACAGCGCCATCGGCAAGATCCAGGAC
AGCCTGTCTAGCACACCATCCGCCCTGGGCAAGCTGCAGGATGTGGTGAATCAGAACGCCCAGGCCCTGAAT
ACCCTGGTGAAGCAGCTGTCCTCTAACTTCGGCGCCATCAGCTCCGTGCTGAATGATATCCTGTCCCGGCTG
GACCCCCCTGAGGCAGAGGTGCAGATCGACCGGCTGATCACAGGCAGACTGCAGAGCCTGCAGACCTACGTG
ACACAGCAGCTGATCAGGGCAGCAGAGATCAGGGCATCCGCCAATCTGGCAGCAACCAAGATGTCTGAGTGC
GTGCTGGGCCAGAGCAAGAGAGTGGACTTCTGCGGCAAGGGCTATCACCTGATGTCCTTTCCTCAGTCTGCC
CCACACGGCGTGGTGTTCCTGCACGIGACCTACGTGCCAGCCCAGGAGAAGAACTTCACCACAGCCCCCGCC
ATCTGTCACGATGGCAAGGCACACTTCCCAAGGGAGGGCGTGTTCGTGAGCAACGGCACCCACTGGTTCGTG
ACACAGAGAAACTICTACGAGCCCCAGATCATCACCACAGACAATACATTCGTGTCTGGCAACTGCGACGTG
GTCATCGGCATCGTGAACAATACCGTGTATGATCCCCTGCAGCCTGAGCTGGACTCCTTCAAGGAGGAGCTG
GATAAGTACTTTAAGAATCACACCTCTCCCGACGTGGATCTGGGCGACATCTCTGGCATCAATGCCAGCGTG
GTGAACATCCAGAAGGAGATCGACAGGCTGAACGAGGIGGCCAAGAATCTGAACGAGAGCCTGATCGATCTG
CAGGAGCTGGGCAAGTACGGCAGCGGCTCCGGCTCTGGCAGCGGCTCCGGCTCTGGCAGCGGCTCCGGCTCT
GGCAGCGGCTCCGGCTCTATGGAGAATACCACAAGCGGCTTTCTGGGACCTCTGCTGGTGCTGCAGGCAGGC
TTCTTTCTGCTGACCCGCATCCTGACAATCCCACAGTCTCTGGATAGCTGGIGGACATCCCTGAACTTCCTG
GGAGGAGCACCAACCTGTCCAGGACAGAATAGCAGGTCCCCCACCTCCAACCACTCTCCTACAAGCTGTCCA
CCCATCTGCCCTGGCTATCGCTGGATGTGCCTGCGGCGGTTCATCATCTTCCTGTTTATCCTGCTGCTGTGC
CTGATCTTCCTGCTGGTGCTGCTGGACTACCAGGGCATGCTGCCCGTGTGCCCACTGCTGCCAGGCACCTCC
ACCACATCTACAGGCCCTTGTAAGACCTGCACAAATCCAGCCAACGGCACCAGCATGTTTCCCTCCTGCTGT
TGCACAAAGCCTTCTGATGGCAATTGTACCTGCATCCCAATCCCCTCTAGCTGGGCCTTTGCCCGGTTCCTG
TGGGAGIGGGCAAGCGTGAGATTCAGCTGGCTGTCCTITCTGGTGCCTTTCGTGCAGTGGTTTGTGGGCCTG
TCTCCAACCGTGTGGCTGAGCGTGATGTGGATGATGTGGTATTGGGGCCCCTCCCTGTACAACATCCTGTCT
CCTTTCCTGCCACTGCTGCCCATCTTCTTTTGCCTGTGGGTGTACATCTGA
S6P-16-HBsAg
(SEQ ID NO: 15)
ATGTTCGTGTTTCTGGTGCTGCTGCCCCTTGTGAGCTCCCAGTGTGTGAACCTGACCACAAGGACCCAGCTG
CCCCCTGCCTATACCAATAGCTTTACAAGGGGCGTGTACTATCCAGACAAGGTGTTCCGCTCTAGCGTGCTG
CACTCCACACAGGATCTGTTCCTGCCCTTCTTTTCTAACGTGACCTGGTTTCACGCCATCCACGTGTCCGGC
ACCAATGGCACAAAGCGGTTTGACAATCCTGTGCTGCCATTCAACGATGGCGTGTACTTTGCCAGCACCGAG
AAGTCCAACATCATCAGAGGCTGGATCTTCGGCACCACACTGGACTCTAAGACACAGAGCCTGCTGATCGTG
AACAATGCCACCAACGTGGTGATCAAAGTGTGTGAGTTCCAGTTTTGCAATGATCCCTTTCTGGGCGTGTAC
TATCACAAGAACAATAAGTCCTGGATGGAGTCTGAGTTCCGCGTGTATTCCTCTGCCAACAATTGCACATTC
GAGTACGTGAGCCAGCCTTTTCTGATGGACCTGGAGGGCAAGCAGGGCAATTTTAAGAACCTGAGGGAGTTC
GTGTTTAAGAATATCGATGGCTATTICAAGATCTACTCCAAGCACACCCCAATCAACCTGGTGCGCGACCTG
CCCCAGGGCTTTTCTGCCCTGGAGCCCCTGGTGGATCTGCCTATCGGCATCAACATCACCCGGTTCCAGACA
CTGCTGGCCCTGCACAGAAGCTACCTGACACCCGGCGACAGCTCCTCTGGATGGACCGCAGGAGCAGCAGCC
TACTATGTGGGCTATCTGCAGCCTAGGACCTTTCTGCTGAAGTACAACGAGAATGGCACCATCACAGACGCC
GTGGATTGTGCCCTGGACCCCCTGTCTGAGACCAAGTGCACACTGAAGAGCTTCACCGTGGAGAAGGGCATC
TATCAGACAAGCAATTTTAGGGTGCAGCCAACCGAGTCCATCGTGCGCTTCCCAAATATCACAAACCTGTGT
CCCTTTGGCGAGGIGTTCAACGCAACCAGGTTCGCAAGCGTGTACGCATGGAATAGGAAGCGCATCTCCAAC
TGCGTGGCCGACTATTCTGTGCTGTACAACTCCGCCTCTTTCAGCACCTTTAAGTGTTATGGCGTGAGCCCC
ACAAAGCTGAATGATCTGTGCTTCACCAACGTGTACGCCGATTCCTTTGTGATCAGGGGAGACGAAGTGAGG
CAGATCGCACCAGGACAGACAGGCAAGATCGCCGACTACAATTATAAGCTGCCCGACGATTTCACCGGCTGC
GTGATCGCCTGGAACAGCAACAATCIGGATTCCAAAGTGGGCGGCAACTACAATTATCTGTACCGGCTGTTC
AGAAAGAGCAATCTGAAGCCTTTTGAGAGGGACATCTCCACAGAGATCTACCAGGCCGGCTCTACCCCATGT
AATGGCGTGGAGGGCTTTAACTGCTATTTCCCTCTGCAGICCTACGGCTTTCAGCCAACCAACGGCGTGGGC
TATCAGCCCTACCGCGTGGTGGTGCTGTCTTTCGAGCTGCTGCACGCACCTGCAACAGTGTGTGGACCAAAG
AAGAGCACCAATCTGGTGAAGAACAAGIGCGTGAACTTCAATTTTAACGGACTGACCGGAACAGGAGTGCTG
ACCGAGAGCAACAAGAAGTTCCTGCCATTTCAGCAGTTCGGCCGGGACATCGCCGATACCACAGACGCCGTG
AGAGACCCTCAGACCCTGGAGATCCTGGATATCACACCATGTTCTTTCGGAGGAGTGAGCGTGATCACACCA
GGAACCAATACATCCAACCAGGTGGCCGTGCTGTATCAGGACGTGAATTGCACCGAGGTGCCAGTGGCAATC
CACGCAGATCAGCTGACCCCTACATGGAGGGTGTACAGCACCGGCTCCAACGTGTTCCAGACAAGGGCAGGA
TGCCTGATCGGAGCAGAGCACGTGAACAATAGCTATGAGTGTGACATCCCAATCGGAGCAGGAATCTGCGCA
TCCTACCAGACCCAGACAAACTCTCCTGGCAGCGCAAGCTCCGTGGCATCCCAGTCTATCATCGCCTATACC
ATGTCTCTGGGCGCCGAGAATAGCGTGGCCTACTCCAACAATTCTATCGCCATCCCCACCAACTTTACAATC
AGCGTGACCACAGAGATCCTGCCTGTGTCCATGACCAAGACATCTGTGGACTGTACAATGTATATCTGCGGC
GATAGCACCGAGTGTTCCAACCTGCTGCTGCAGTACGGCTCCTTCTGCACCCAGCTGAATAGGGCACTGACA
GGAATCGCAGTGGAGCAGGACAAGAACACACAGGAGGTGTTTGCCCAGGTGAAGCAGATCTACAAGACCCCA
CCCATCAAGGACTTCGGCGGCTTCAATTTTAGCCAGATCCTGCCTGATCCAAGCAAGCCATCCAAGCGGTCT
CCCATCGAGGACCTGCTGTTCAACAAGGTGACCCTGGCCGATGCCGGCTTTATCAAGCAGTATGGCGATTGT
CTGGGCGACATCGCCGCCAGAGACCTGATCTGCGCCCAGAAGTTTAATGGCCTGACCGTGCTGCCTCCACTG
CTGACAGATGAGATGATCGCCCAGTACACATCTGCACTGCTGGCAGGAACCATCACAAGCGGATGGACCTTC
GGAGCAGGACCTGCACTGCAGATCCCCTTCCCTATGCAGATGGCCTATAGATTTAACGGCATCGGCGTGACC
CAGAATGTGCTGTACGAGAACCAGAAGCTGATCGCCAATCAGTTTAACAGCGCCATCGGCAAGATCCAGGAC
TCCCTGTCTAGCACACCATCTGCCCTGGGCAAGCTGCAGGATGTGGTGAATCAGAACGCCCAGGCCCTGAAT
ACCCTGGTGAAGCAGCTGTCCTCTAACTTCGGCGCCATCAGCTCCGTGCTGAATGATATCCTGTCCCGGCTG
GACCCCCCTGAGGCAGAGGTGCAGATCGACCGGCTGATCACAGGCAGACTGCAGTCCCTGCAGACCTACGTG
ACACAGCAGCTGAICAGGGCAGCAGAGATCAGGGCATCTGCAAATCTGGCCGCCACCAAGATGAGCGAGTGT
GTGCTGGGCCAGTCCAAGAGAGTGGACTTCTGCGGCAAGGGCTATCACCTGATGTCTTTTCCTCAGAGCGCA
CCACACGGAGTGGTGTTCCTGCACGIGACCTACGIGCCAGCCCAGGAGAAGAACTTCACCACAGCCCCCGCC
ATCTGTCACGATGGAAAGGCACACTTCCCAAGGGAGGGAGTGTTTGTGTCTAACGGCACCCACIGGITCGTG
ACACAGAGAAATTITTATGAGCCCCAGATCATCACCACAGACAATACATTCGTGTCCGGCAACTGCGATGTG
GTGATCGGCATCGIGAACAATACCGTGTATGATCCCCTGCAGCCTGAGCTGGACTCITTCAAGGAGGAGCTG
GATAAGTACTTTAAGAATCACACCAGCCCCGACGTGGATCTGGGCGACATCAGCGGCATCAATGCCTCCGTG
GTGAACATCCAGAAGGAGATCGACCGGCTGAACGAGGTGGCCAAGAATCTGAACGAGAGCCTGATCGATCTG
CAGGAGCTGGGAAAGTACGGCAGCGGCTCCGGCTCTGGCAGCGGCTCCGGCTCTGGCAGCGGCTCCGGCTCT
GGCAGCGGCTCCGGCTCTGGCAGCGGCTCCGGCTCTGGCAGCATGGAGAATACCACAAGCGGCTTTCTGGGA
CCTCTGCTGGTGCTGCAGGCAGGCTTCTTTCTGCTGACCAGAATCCTGACAATCCCACAGAGCCTGGATTCC
TGGTGGACATCCCTGAACTTCCTGGGAGGAGCACCAACCTGTCCAGGACAGAATTCCAGGTCTCCCACCTCT
AACCACAGCCCTACATCCTGTCCACCCATCTGCCCTGGCTATCGCTGGATGTGTCTGCGGAGATTTATCATC
TTCCTGITTATCCTGCTGCTGTGCCTGATCTTCCTGCIGGTGCTGCTGGACTACCAGGGCATGCTGCCTGTG
TGCCCACTGCTGCCAGGAACCTCTACCACAAGCACAGGCCCTTGTAAGACCTGCACAAATCCAGCCAACGGC
ACCTCCATGTTTCCCTCTTGCTGTTGCACAAAGCCTAGCGATGGCAATTGTACCTGCATCCCAATCCCCICT
AGCTGGGCCTTTGCACGGTTCCTGTGGGAGTGGGCATCCGTGAGATTCTCCIGGCTGTCTTTTCTGGTGCCT
TTCGTGCAGTGGTTTGTGGGCCTGAGCCCAACCGTGTGGCTGTCCGTGATGIGGATGATGTGGTATTGGGGC
CCCTCTCTGTACAACATCCTGAGCCCTTTCCTGCCACTGCTGCCCATCTTCTTTTGCCTGTGGGTGTACATC
TGA

In some aspects, the nucleic acid molecule encoding the fusion protein as described herein comprises or consists of a DNA sequence at least 80% (such as at least 90%, at least 95%, at least 96%, at least 97%, at least 98%, or at least 99%) identical to SEQ ID NO: 13 that encodes a fusion protein set forth as SEQ ID NO: 10, or the complement thereof, or a corresponding RNA sequence or the complement thereof. In some aspects, the nucleic acid encoding the fusion protein as described herein comprises or consists of a DNA sequence set forth as SEQ ID NO: 13, or the complement thereof, or a corresponding RNA sequence or the complement thereof.

In some aspects, the nucleic acid molecule encoding the fusion protein as described herein comprises or consists of a DNA sequence at least 80% (such as at least 90%, at least 95%, at least 96%, at least 97%, at least 98%, or at least 99%) identical to SEQ ID NO: 14 that encodes a fusion protein set forth as SEQ ID NO: 11, or the complement thereof, or a corresponding RNA sequence or the complement thereof. In some aspects, the nucleic acid encoding the fusion protein as described herein comprises or consists of a DNA sequence set forth as SEQ ID NO: 14, or the complement thereof, or a corresponding RNA sequence or the complement thereof.

In some aspects, the nucleic acid molecule encoding the fusion protein as described herein comprises or consists of a DNA sequence at least 80% (such as at least 90%, at least 95%, at least 96%, at least 97%, at least 98%, or at least 99%) identical to SEQ ID NO: 15 that encodes a fusion protein set forth as SEQ ID NO: 12, or the complement thereof, or a corresponding RNA sequence or the complement thereof. In some aspects, the nucleic acid encoding the fusion protein as described herein comprises or consists of a DNA sequence set forth as SEQ ID NO: 15, or the complement thereof, or a corresponding RNA sequence or the complement thereof.

### B. Nucleic Acid Molecules Encoding Coronavirus S Ectodomain—HBsAg Fusion Proteins

Also disclosed herein are nucleic acid molecules encoding a fusion protein comprising a recombinant Coronavirus S ectodomain other than a SARS-CoV-2 S ectodomain, such as SARS-CoV-1, MERS-CoV, 229E, NL63, OC43, or HKU1 S ectodomain, fused via a peptide linker to a HBsAg protein. Briefly, the recombinant Coronavirus S ectodomain is swapped with the SARS-CoV-2 S ectodomain in the fusion protein described above. When expressed in mammalian cells (for example, by administration to a mammalian subject), the fusion protein self-assembles to form a HBsAg nanoparticle with Coronavirus S ectodomain trimers (e.g., SARS-CoV-1, MERS-CoV, 229E, NL63, OC43, or HKU1 S ectodomain trimers) extending radially outward from an outer surface of the HBsAg nanoparticle. The Coronavirus S ectodomain (such as SARS-CoV-1, MERS-CoV, 229E, NL63, OC43, or HKU1 S ectodomain) can be constructed by swapping the Coronavirus S ectodomain (such as SARS-CoV-1, MERS-CoV, 229E, NL63, OC43, or HKU1 S ectodomain) for the SARS-CoV-2 ectodomain in the SARS-CoV-2-HBsAg fusion proteins described herein.

The recombinant Coronavirus S ectodomain (such as SARS-CoV-1, MERS-CoV, 229E, NL63, OC43, or HKU1 S ectodomain) included in the fusion protein is stabilized in a prefusion conformation by one or more amino acid substitutions. In some aspects, the recombinant Coronavirus S ectodomain (such as SARS-CoV-1, MERS-CoV, 229E, NL63, OC43, or HKU1 S ectodomain) included in the fusion protein is stabilized in a prefusion conformation by the “6P” mutations (e.g., for SARS-CoV-1, MERS-CoV S) or “5P” mutations (e.g., for 229E, NL63, OC43 and HKU1 S). The “6P” proline mutations are equivalent to F817P, A892P, A899P, A942P, K986P, and V987P relative to the SARS-CoV-2 sequence provided as SEQ ID NO: 1. The “5P” proline mutations are equivalent to F817P, A899P, A942P, K986P, and V987P relative to the SARS-CoV-2 sequence provided as SEQ ID NO: 1. The locations of the relevant positions for the substitutions can be identified by sequence and structure alignment of the Coronavirus S ectodomain (e.g., SARS-CoV-1, MERS-CoV, 229E, NL63, OC43, or HKU1 S ectodomain) with the corresponding SARS-CoV-2 S ectodomain set forth as SEQ ID NO: 1; further, examples of SARS-CoV-1, MERS-CoV, 229E, NL63, OC43, and HKU1 S ectodomain sequences with the “6P” or “5P” mutations are provided herein.

In some aspects, the recombinant SARS-CoV-1 S ectodomain included in the fusion protein is a single-chain SARS-CoV-1 S ectodomain including a mutation of the S1/S2 protease cleavage site to prevent cleavage and formation of distinct S1 and S2 polypeptide chains. Examples of the mutation can be but are not limited to glycine, serine or alanine substitution. In some aspects, the S1 and S2 polypeptides in the single chain SARS-CoV-1 S ectodomain are joined by a linker, such as a peptide linker. Examples of peptide linkers that can be used include glycine, serine, and glycine-serine linkers. Examples of SARS-CoV-1, MERS-CoV, 229E, NL63, OC43, and HKU1 S ectodomain sequences containing a mutated S1/S2 with or without a mutated S2 cleavage site are provided herein. Any of the prefusion stabilizing mutations (or combinations thereof) disclosed herein can be included in the single chain SARS-CoV-2 S ectodomain.

>SARS S6P ectodomain,
SEQ ID NO: 322
MFIFLLFLTLTSGSDLDRCTTEDDVQAPNYTQHTSSMRGVYYPDEIFRSDTLYLTQDLFLPFYSNVTGEHTI
NHTFGNPVIPFKDGIYFAATEKSNVVRGWVFGSTMNNKSQSVIIINNSTNVVIRACNFELCDNPFFAVSKPM
GTQTHTMIFDNAFNCTFEYISDAFSLDVSEKSGNFKHLREFVEKNKDGFLYVYKGYQPIDVVRDLPSGENTL
KPIFKLPLGINITNFRAILTAFSPAQDIWGTSAAAYFVGYLKPTTFMLKYDENGTITDAVDCSQNPLAELKC
SVKSFEIDKGIYQTSNFRVVPSGDVVRFPNITNLCPFGEVENATKFPSVYAWERKKISNCVADYSVLYNSTE
FSTFKCYGVSATKLNDLCFSNVYADSFVVKGDDVRQIAPGQTGVIADYNYKLPDDEMGCVLAWNTRNIDATS
TGNYNYKYRYLRHGKLRPFERDISNVPFSPDGKPCTPPALNCYWPLNDYGFYTTTGIGYQPYRVVVLSFELL
NAPATVCGPKLSTDLIKNQCVNFNENGLIGTGVLTPSSKRFQPFQQFGRDVSDFTDSVRDPKTSEILDISPC
AFGGVSVITPGTNASSEVAVLYQDVNCTNVSAAIHADQLTPAWRIYSTGNNVFQTQAGCLIGAEHVDTSYEC
DIPIGAGICASYHTVSLLRSTSQKSIVAYTMSLGADSSIAYSNNTIAIPTNFSISITTEVMPVSMAKTSVDC
NMYICGDSTECANLLLQYGSFCTQLNRALSGIAAEQDRNTREVFAQVKQMYKTPTLKYFGGFNFSQILPDPL
KPTKRSPIEDLLFNKVTLADAGEMKQYGECLGDINARDLICAQKENGLTVLPPLLIDDMIAAYTAALVSGTA
TAGWTFGAGPALQIPFPMQMAYRENGIGVTQNVLYENQKQIANQFNKAISQIQESLITTPTALGKLQDVVNQ
NAQALNTLVKQLSSNFGAISSVLNDILSRLDPPEAEVQIDRLITGRLQSLQTYVTQQLIRAAEIRASANLAA
TKMSECVLGQSKRVDFCGKGYHLMSFPQAAPHGVVFLHVTYVPSQERNFTTAPAICHEGKAYFPREGVEVEN
GTSWFITQRNFFSPQIITTDNTFVSGNCDVVIGIINNTVYDPLQPELDSFKGELDKYFKNHTSPDVDLGDIS
GINASVVNIQKEIDRLNEVAKNLNESLIDLQELGKY
>MERS S6P ectodomain,
SEQ ID NO: 323
MIHSVFLLMFLLTPTESYVDVGPDSVKSACIEVDIQQTFFDKTWPRPIDVSKADGIIYPQGRTYSNITITYQ
GLFPYQGDHGDMYVYSAGHATGTTPQKLFVANYSQDVKQFANGFVVRIGAAANSIGTVIISPSTSATIRKIY
PAFMLGSSVGNFSDGKMGRFFNHTLVLLPDGCGTLLRAFYCILEPRSGNHCPAGNSYTSFATYHTPATDCSD
GNYNRNASLNSFKEYFNLRNCTFMYTYNITEDEILEWFGITQTAQGVHLFSSRYVDLYGGNMFQFATLPVYD
TIKYYSIIPHSIRSIQSDRKAWAAFYVYKLQPLTELLDESVDGYIRRAIDCGENDLSQLHCSYESEDVESGV
YSVSSFEAKPSGSVVEQAEGVECDESPLLSGTPPQVYNFKRLVFINCNYNLTKLLSLFSVNDFTCSQISPAA
IASNCYSSLILDYFSYPLSMKSDLSVSSAGPISQFNYKQSFSNPTCLILATVPHNLTTITKPLKYSYINKCS
RLLSDDRTEVPQLVNANQYSPCVSIVPSTVWEDGDYYRKQLSPLEGGGWLVASGSTVAMTEQLQMGFGITVQ
YGTDTNSVCPKLEFANDTKIASQLGNCVEYSLYGVSGRGVFQNCTAVGVRQQRFVYDAYQNLVGYYSDDGNY
YCLRACVSVPVSVIYDKETKTHATLFGSVACEHISSTMSQYSRSTRSMLKRRDSTYGPLQTPVGCVLGLVNS
SLFVEDCKLPLGQSLCALPDTPSTLTPRSVRVPGEMRLASIAFNHPIQVDQLNSSYFKLSIPTNFSFGVTQE
YIQTTIQKVTVDCKQYVCNGFQKCEQLLREYGQFCSKINQALHGANLRQDDSVRNLFASVKSSQSSPIIPGE
GGDENLILLEPVSISTGSRSAPSAIEDLLEDKVTIADPGYMQGYDDCMQQGPASARDLICAQYVAGYKVLPP
LMDVNMEAAYTSSLLGSIAGVGWPAGLSSFAAPPFAQSIFYRLNGVGITQQVLSENQKLIANKENQALGAMQ
TGFTTTPEAFQKVQDAVNNNAQALSKLASELSNTFGAISASIGDIIQRLDPPEQDAQIDRLINGRLTTLNAF
VAQQLVRSESAALSAQLAKDKVNECVKAQSKRSGFCGQGTHIVSEVVNAPNGLYFMHVGYYPSNHIEVVSAY
GLCDAANPTNCIAPVNGYFIKTNNTRIVDEWSYTGSSFYAPEPITSLNTKYVAPQVTYQNISTNLPPPLLGN
STGIDFQDELDEFFKNVSTSIPNFGSLTQINTILLDLTYEMLSLQQVVKALNESYIDLKELGNYTYYNK
>229E (GenBank: ABB90529.1) S5P ectodomain,
SEQ ID NO: 324
MFVLLVAYALLHIAGCQTTNGMNTSHSVCNGCVGHSENVFAVESGGYIPSNFAFNNWELLINTSSVVDGVVR
SFQPLLLNCLWSVSGSRLTTGFVYFNGTGRGACKGFYSNASSDVIRYNINFEENLRRGTILFKTSYGAVVFY
CTNNTLVSGDAHIPSGTVLGNFYCFVNITIGNETTSAFVGALPKIVREFVISRTGHFYINGYRYFSLGNVEA
VNFNVTNAATTVCTVALASYADVLVNVSQTAIANIIYCNSVINRLRCDQLSFDVPDGFYSTSPIQSVELPVS
IVSLPVYHKHTFIVLYVNFELRRGPGRCYNCRPAVVNITLANFNETKGPLCVDTSHFTTQFFGVKFDRWSAS
INTGNCPFSFGKVNNFVKFGSVCFSLKDIPGGCAMPIMANLANLNSHNIGSLYVSWSDGDVITGVPKPVEGV
SSFMNVTLNKCTKYNIYDVSGVGVIRISNDTFLNGITYTSTSGNLLGFKDVINGTIYSITPCNPPDQLVVYQ
QAVVGAMLSENFTSYGESNVVEMPKFFYASNGTYNCTDAVLTYSSFGVCADGSIIAVQPRNVSYDSVSAIVT
ANLSIPSNWTTSVQVEYLQITSTPIVVDCSTYVCNGNVRCVELLKQYTSACKTIEDALRNSAMLESADVSEM
LTFDKKAFTLANVSSFGDYNLSSVIPSLPRSGSRVPGRSAIEDILFSKLVTSGLGTVDADYKKCTKGLSIAD
LACAQYYNGIMVLPGVADAERMAMYTGSLIGGIALGGLTSPASIPFSLAIQSRLNYVALQTDVLQENQKILA
ASFNKAMTNIVDAFTGVNDPITQTSQALQTVATALNKIQDVVNQQGNSLNHLTSQLRQNFQAISSSIQAIYD
RLDPPQADQQVDRLITGRLAALNVFVSHTLTKYTEVRASRQLAQQKVNECVKSQSKRYGFCGNGTHIFSLVN
AAPEGLVFLHTVLLPTQYKDVEAWSGLCVDGRNGYVLRQPNLALYKEGNYYRITSRIMFEPRIPTIADEVQI
ENCNVTFVNISRSELQTIVPEYIDVNKTLQELSYKLPNYTVPDLVVEQYNQTILNLTSEISTLENKSAELNY
TVQKLQTLIDNINSTLVDLKWLNRVETYIKWP
>NL63 (GenBank: APF29063.1) S5P ectodomain,
SEQ ID NO: 325
MKLFLILLVLPLASCFFTCNSNANLSMLQLGVPDNSSTIVTGLLPTHWECANQSTSVYSANGFFYIDVGNHR
SAFALHTGYYDVNQYYIYVINEIGLNASVTLKICKFGINTTFDFLSNSSSSFDCIVNLLFTEQLGAPLGITI
SGETVRLHLYNVTRTFYVPAAYKLIKLSVKCYFNYSCVFSVVNATVTVNVTTHNGRVVNYTVCDDCNGYTDN
IFSVQQDGRIPNGFPFNNWFLLINGSTLVDGVSRLYQPLRLTCLWPVPGLKSSTGFVYFNATGSDVNCNGYQ
HNSVADVMRYNLNFSANSVDNLKSGVIVEKTLQYDVLFYCSNSSSGVLDTTIPFGPSSQPYYCFINSTINTT
HVSTFVGILPPTVREIVVARTGQFYINGFKYFDLGFIEAVNENVITASATDFWTVAFATFVDVLVNVSATKI
QNLLYCDSPFEKLQCEHLQFGLQDGEYSANFLDDNVLPETYVALPIYYQHTDINFTATASFGGSCYVCKPHQ
VNISLNGNTSVCVRTSHFSIRYIYNRVKSGSPGDSSWHIYLKSGTCPFSFSKLNNFQKFKTICESTVEVPGS
CNFPLEATWHYTSYTIVGALYVTWSEGNSITGVPYPVSGIREFSNLVLNNCTKYNIYDYVGTGIIRSSNQSL
AGGITYVSNSGNLLGFKNVSTGNIFIVTPCNQPDQVAVYQQSIIGAMTAVNESRYGLQNLLQLPNFYYVSNG
GNNCTTAVMTYSNFGICADGSLIPVRPRNSSDNGISAIITANLSIPSNWTTSVQVEYLQITSTPIVVDCATY
VCNGNPRCKNLLKQYTSACKTIEDALRLSAHLETNDVSSMLTFDSNAFSLANVTSFGDYNLSSVLPQRNIRS
SRIAGRSPLEDLLESKVVTSGLGTVDVDYKSCTKGLSIADLACAQYYNGIMVLPGVADAERMAMYTGSLIGG
MVLGGLISAPAIPFSLALQARLNYVALQTDVLQENQKILAASENKAINNIVASFSSVNDPITQTAEAIHTVT
IALNKIQDVVNQQGSALNHLTSQLRHNFQAISNSIQAIYDRLDPPQADQQVDRLITGRLAALNAFVSQVLNK
YTEVRSSRRLAQQKVNECVKSQSNRYGFCGNGTHIFSIVNSAPDGLLFLHTVLLPTDYKNVKAWSGICVDGI
YGYVLRQPNLVLYSDNGVFRVTSRIMFQPRLPVLSDFVQIYNCNVTFVNISRVELHTVIPDYVDVNKTLQEF
AQNLPKYVKPNFDLTPFNLTYLNLSSELKQLEAKTASLFQTTVELQGLIDQINSTYVDLKLLNRFENYIKWP
>OC43 (GenBank: AIX10763.1) S5P ectodomain,
SEQ ID NO: 326
MFLILLISLPTAFAVIGDLNCPLDPKLKGSENNRDIGSPSISTDTVDVINGLGTYYVLDRVYLNTTLFLNGY
YPTSGSTYRNMALKGTDLLSTLWFKPPFLSDFINGIFAKVKNTKVFKDGVMYSEFPAITIGSTEVNTSYSVV
VQPRTINSTQDGVNKLQGLLEVSVCQYNMCEHPHTICHPNLGNHEKELWHLDTGVVSCLYKRNETYDVNATY
LYFHFYQEGGTFYAYFTDTGFVTKFLFNVYLGMALSHYYVMPLTCISRRDIGFTLEYWVTPLTPRQYLLAEN
QDGIIFNAVDCMSDFMSEIKCKTQSIAPPTGVYELNGYTVQPIADVYRRKPDLPNCNIEAWLNDKSVPSPLN
WERKTFSNCNFNMSSLMSFIQADSFTCNNIDAAKIYGMCFSSITIDKFAIPNRRKVDLQLGNLGYLQSSNYR
IDTTATSCQLYYNLPAANVSVSRENPSTWNKRFGFIEDSVFVPQPTGVFTNHSVVYAQHCFKAPKNFCPCSS
CPGKNNGIGTCPAGINSLTCDNLCTLDPITFKAPDTYKCPQTKSLVGIGEHCSGLAVKSDYCGNNSCTCQPQ
AFLGWSADSCLQGDKCNIFANFILHDVNNGLTCSTDLQKANTEIELGVCVNYDLYGISGQGIFVEVNATYYN
SWQNLLYDSNGNLYGFRDYITNRTFMIHSCYSGRVSAAYHANSPEPALLFRNIKCNYVENNSLTRQLQPINY
SFDSYLGCVVNAYNSTAISVQTCDLTVGSGYCVDYSKNRRSRRAITTGYRFTNFEPFTVNSVNDSLEPVGGL
YEIQIPSEFTIGNMEEFIQTSSPKVTIDCAAFVCGDYAACKLQLVEYGSFCDNINAILTEVNELLDTTQLQV
ANSLMNGVTLSTKLKDGVNFNVDDINFSPVLGCLGSECSKPSSRSAIEDLLEDKVKLSDVGFVEAYNNCTGG
AEIRDLICVQSYKGIKVLPPLLSENQISGYTLAATSASLFPPWTAPAGVPFYLNVQYRINGLGVTMDVLSQN
QKLIANAFNNALHAIQQGEDPTNSALVKIQAVVNANSEALNNLLQQLSNRFGAISASLQEILSRLDPPEAEA
QIDRLINGRLTALNAYVSQQLSDSTLVKFSAAQAMEKVNECVKSQSSRINFCGNGNHIISLVQNAPYGLYFI
HFNYVPTKYVTAKVSPGLCIAGNRGIAPKSGYFVNVNNTWMYTGSGYYYPEPITENNVVVMSTCAVNYTKAP
YVMLNTSIPNLPDFKEELGQWFKNQTSVAPDLSLDYINVTFLDLQVEMNRLQEAIKVLNHSYINLKDIGTYE
YYVKWP
>HKU1 (GenBank: AGW27881.1) S5P ectodomain,
SEQ ID NO: 327
MLLIIFILPTTLAVIGDENCTNFAINDKNTTVPRISEYVVDVSYGLGTYYILDRVYLNTTILFTGYFPKSGA
NERDLSLKGTTYLSTLWYQKPFLSDENNGIFSRVKNTKLYVNKTLYSEFSTIVIGSVFINNSYTIVVQPHNG
VLEITACQYTMCEYPHTICKSKGSSRNESWHFDKSEPLCLFKKNFTYNVSTDFLYFHFYQERGTFYAYYADS
GMPTTFLESLYLGILLSHYYVLPLTCNAISSNTDNETLQYWVTPLSKRQYLLKFDNRGVITNAVDCSSSFFS
EIQCKTKSLLPNTGVYDLSGFTVKPVATVHRRIPDLPDCDIDKWLNNENVPSPLNWERKIFSNCNENLSTLL
RLVHTDSFSCNNFDESKIYGSCFKSIVLDKFAIPNSRRSDLQLGSSGFLQSSNYKIDTTSSSCQLYYSLPAI
NVTINNYNPSSWNRRYGENNENLSSHSVVYSRYCFSVNNTFCPCAKPSFASSCKSHKPPSASCPIGINYRSC
ESTTVLDHTDWCRCSCLPDPITAYDPRSCSQKKSLVGVGEHCAGFGVDEEKCGVLDGSYNVSCLCSTDAFLG
WSYDTCVSNNRCNIFSNFILNGINSGTTCSNDLLQPNTEVYTDVCVDYDLYGITGQGIFKEVSAVYYNSWQN
LLYDSNGNIIGFKDFVTNKTYNIFPCYAGRVSAAFHQNASSLALLYRNLKCSYVLNNISLATQPYEDSYLGC
VFNADNLTDYSVSSCALRMGSGFCVDYNSPSESSSRRKRRSISASYRFVTFEPFNVSFVNDSIESVGGLYEI
KIPTNFTIVGQEEFIQTNSPKVTIDCSLFVCSNYAACHDLLSEYGTFCDNINSILDEVNGLLDITQLHVADT
LMQGVTLSSNLNTNLHEDVDNINFKSLVGCLGPHCGSPSRSFFEDLLEDKVKLSDVGFVEAYNNCTGGSEIR
DLLCVQSENGIKVLPPILSESQISGYTTAATVAAMFPPWSAPAGIPFSLNVQYRINGLGVTMDVLNKNQKLI
ATAFNNALLSIQNGFSPINSALAKIQSVVNSNAQALNSLLQQLENKFGAISSSLQEILSRLDPPEAQVQIDR
LINGRLTALNAYVSQQLSDISLVKFGAALAMEKVNECVKSQSPRINFCGNGNHILSLVQNAPYGLLEMHESY
KPISFKTVLVSPGLCISGDVGIAPKQGYFIKHNDHWMFTGSSYYYPEPISDKNVVEMNTCSVNFTKAPLVYL
NHSVPKLSDFESELSHWFKNQTSIAPNLILNLHTINATELDLYYEMNLIQESIKSLNNSYINLKDIGTYEMY
VKWP

In some aspects, the Coronavirus S ectodomain further includes one or more mutations to eliminate protease cleavage sites. Exemplary sequences of recombinant SARS-CoV-1, MERS-CoV, 229E, NL63, OC43, and HKU′1 S proteins including the 6P or 5P substitutions for prefusion stabilization and S1/S2 with or without S2 protease cleavage site mutations for inclusion in the fusion protein are provided as:

>SARS-COV-1 S6P,
SEQ ID NO: 328
MFIFLLFLTLTSGSDLDRCTTFDDVQAPNYTQHTSSMRGVYYPDEIFRSDTLYLTQDLFLPFYSNVTGEHTI
NHTFGNPVIPFKDGIYFAATEKSNVVRGWVFGSIMNNKSQSVIIINNSINVVIRACNFELCDNPFFAVSKPM
GTQTHTMIFDNAFNCTFEYISDAFSLDVSEKSGNEKHLREFVEKNKDGFLYVYKGYQPIDVVRDLPSGENTL
KPIFKLPLGINIINFRAILTAFSPAQDIWGTSAAAYFVGYLKPTTFMLKYDENGTITDAVDCSQNPLAELKC
SVKSFEIDKGIYQTSNERVVPSGDVVRFPNITNLCPFGEVENATKFPSVYAWERKKISNCVADYSVLYNSTE
FSTFKCYGVSATKLNDLCFSNVYADSFVVKGDDVRQIAPGQTGVIADYNYKLPDDEMGCVLAWNTRNIDATS
TGNYNYKYRYLRHGKLRPFERDISNVPFSPDGKPCTPPALNCYWPLNDYGFYTTTGIGYQPYRVVVLSFELL
NAPATVCGPKLSTDLIKNQCVNFNFNGLTGTGVLTPSSKRFQPFQQFGRDVSDFTDSVRDPKISEILDISPC
AFGGVSVITPGTNASSEVAVLYQDVNCTNVSAAIHADQLTPAWRIYSTGNNVFQTQAGCLIGAEHVDTSYEC
DIPIGAGICASYHTVSLLSSTSQKSIVAYTMSLGADSSIAYSNNIIAIPINESISITTEVMPVSMAKTSVDC
NMYICGDSTECANLLLQYGSFCTQLNRALSGIAAEQDRNTREVFAQVKQMYKTPTLKYFGGENFSQILPDPL
KPTKRSPIEDLLFNKVTLADAGFMKQYGECLGDINARDLICAQKENGLIVLPPLLIDDMIAAYTAALVSGTA
TAGWTFGAGPALQIPFPMQMAYRENGIGVTQNVLYENQKQIANQFNKAISQIQESLITTPTALGKLQDVVNQ
NAQALNTLVKQLSSNFGAISSVLNDILSRLDPPEAEVQIDRLITGRLQSLQTYVTQQLIRAAEIRASANLAA
TKMSECVLGQSKRVDFCGKGYHLMSFPQAAPHGVVFLHVTYVPSQERNETTAPAICHEGKAYFPREGVEVEN
GTSWFITQRNFFSPQIITTDNTFVSGNCDVVIGIINNTVYDPLQPELDSEKGELDKYFKNHTSPDVDLGDIS
GINASVVNIQKEIDRLNEVAKNLNESLIDLQELGKY
>MERS-COV S6P,
SEQ ID NO: 329
MIHSVFLLMFLLTPTESYVDVGPDSVKSACIEVDIQQTFFDKTWPRPIDVSKADGIIYPQGRTYSNITITYQ
GLFPYQGDHGDMYVYSAGHATGTTPQKLFVANYSQDVKQFANGFVVRIGAAANSIGTVIISPSTSATIRKIY
PAFMLGSSVGNFSDGKMGRFFNHTLVLLPDGCGTLLRAFYCILEPRSGNHCPAGNSYTSFATYHTPATDCSD
GNYNRNASLNSFKEYFNLRNCTFMYTYNITEDEILEWFGITQTAQGVHLESSRYVDLYGGNMFQFATLPVYD
TIKYYSIIPHSIRSIQSDRKAWAAFYVYKLQPLTFLLDESVDGYIRRAIDCGENDLSQLHCSYESEDVESGV
YSVSSFEAKPSGSVVEQAEGVECDFSPLLSGTPPQVYNFKRLVFINCNYNLIKLLSLFSVNDFTCSQISPAA
IASNCYSSLILDYFSYPLSMKSDLSVSSAGPISQFNYKQSFSNPICLILATVPHNLITITKPLKYSYINKCS
RLLSDDRTEVPQLVNANQYSPCVSIVPSTVWEDGDYYRKQLSPLEGGGWLVASGSTVAMTEQLQMGFGITVQ
YGTDTNSVCPKLEFANDTKIASQLGNCVEYSLYGVSGRGVFQNCTAVGVRQQRFVYDAYQNLVGYYSDDGNY
YCLRACVSVPVSVIYDKETKTHATLEGSVACEHISSTMSQYSRSTRSMLKRRDSTYGPLQTPVGCVLGLVNS
SLFVEDCKLPLGQSLCALPDTPSTLTPASVGSVPGEMRLASIAFNHPIQVDQLNSSYFKLSIPTNFSFGVTQ
EYIQTTIQKVTVDCKQYVCNGFQKCEQLLREYGQFCSKINQALHGANLRQDDSVRNLFASVKSSQSSPIIPG
FGGDENLILLEPVSISTGSASAPSAIEDLLFDKVTIADPGYMQGYDDCMQQGPASARDLICAQYVAGYKVLP
PLMDVNMEAAYTSSLLGSIAGVGWPAGLSSFAAPPFAQSIFYRLNGVGITQQVLSENQKLIANKENQALGAM
QTGFTTTPEAFQKVQDAVNNNAQALSKLASELSNIFGAISASIGDIIQRLDPPEQDAQIDRLINGRLTTLNA
FVAQQLVRSESAALSAQLAKDKVNECVKAQSKRSGFCGQGTHIVSFVVNAPNGLYFMHVGYYPSNHIEVVSA
YGLCDAANPTNCIAPVNGYFIKINNTRIVDEWSYTGSSFYAPEPITSLNTKYVAPQVTYQNISINLPPPLLG
NSTGIDFQDELDEFFKNVSTSIPNFGSLTQINTTLLDLTYEMLSLQQVVKALNESYIDLKELGNYTYYNK
>229E S5P (Based on GenBank: ABB90529.1),
SEQ ID NO: 330
MFVLLVAYALLHIAGCQTTNGMNTSHSVCNGCVGHSENVFAVESGGYIPSNFAFNNWFLLTNTSSVVDGVVR
SFQPLLLNCLWSVSGSRLTTGFVYFNGTGRGACKGFYSNASSDVIRYNINFEENLRRGTILFKTSYGAVVFY
CTNNTLVSGDAHIPSGTVLGNFYCFVNTTIGNETTSAFVGALPKTVREFVISRTGHFYINGYRYFSLGNVEA
VNFNVTNAATTVCTVALASYADVLVNVSQTAIANIIYCNSVINRLRCDQLSEDVPDGFYSTSPIQSVELPVS
IVSLPVYHKHIFIVLYVNFELRRGPGRCYNCRPAVVNITLANFNETKGPLCVDTSHFTTQFFGVKFDRWSAS
INTGNCPFSFGKVNNFVKFGSVCFSLKDIPGGCAMPIMANLANLNSHNIGSLYVSWSDGDVITGVPKPVEGV
SSFMNVTLNKCTKYNIYDVSGVGVIRISNDTFLNGITYTSTSGNLLGFKDVINGTIYSITPCNPPDQLVVYQ
QAVVGAMLSENFTSYGFSNVVEMPKFFYASNGTYNCTDAVLTYSSFGVCADGSIIAVQPGNVSYDSVSAIVT
ANLSIPSNWTTSVQVEYLQITSTPIVVDCSTYVCNGNVRCVELLKQYTSACKTIEDALRNSAMLESADVSEM
LTFDKKAFTLANVSSFGDYNLSSVIPSLPRSGSGVPGSSAIEDILFSKLVTSGLGTVDADYKKCTKGLSIAD
LACAQYYNGIMVLPGVADAERMAMYTGSLIGGIALGGLISPASIPESLAIQSRLNYVALQTDVLQENQKILA
ASFNKAMTNIVDAFTGVNDPITQTSQALQTVATALNKIQDVVNQQGNSLNHLTSQLRQNFQAISSSIQAIYD
RLDPPQADQQVDRLITGRLAALNVFVSHTLTKYTEVRASRQLAQQKVNECVKSQSKRYGFCGNGTHIFSLVN
AAPEGLVFLHTVLLPTQYKDVEAWSGLCVDGRNGYVLRQPNLALYKEGNYYRITSRIMFEPRIPTIADFVQI
ENCNVTEVNISRSELQTIVPEYIDVNKTLQELSYKLPNYTVPDLVVEQYNQTILNLTSEISTLENKSAELNY
TVQKLQTLIDNINSTLVDLKWLNRVETYIKWP
>NL63 (GenBank: APF29063.1),
SEQ ID NO: 331
MKLFLILLVLPLASCEFTCNSNANLSMLQLGVPDNSSTIVTGLLPTHWFCANQSTSVYSANGFFYIDVGNHR
SAFALHTGYYDVNQYYIYVTNEIGLNASVTLKICKFGINTTFDFLSNSSSSFDCIVNLLFTEQLGAPLGITI
SGETVRLHLYNVTRTFYVPAAYKLTKLSVKCYFNYSCVFSVVNATVTVNVTTHNGRVVNYTVCDDCNGYTDN
IFSVQQDGRIPNGFPFNNWELLINGSTLVDGVSRLYQPLRLTCLWPVPGLKSSTGFVYFNATGSDVNCNGYQ
HNSVADVMRYNLNFSANSVDNLKSGVIVEKTLQYDVLFYCSNSSSGVLDTTIPFGPSSQPYYCFINSTINTT
HVSTFVGILPPTVREIVVARTGQFYINGFKYFDLGFIEAVNENVITASATDEWTVAFATFVDVLVNVSATKI
QNLLYCDSPFEKLQCEHLQFGLQDGFYSANFLDDNVLPETYVALPIYYQHTDINFTATASFGGSCYVCKPHQ
VNISLNGNTSVCVRTSHESIRYIYNRVKSGSPGDSSWHIYLKSGTCPFSFSKLNNFQKFKTICESTVEVPGS
CNFPLEATWHYTSYTIVGALYVTWSEGNSITGVPYPVSGIREFSNLVLNNCTKYNIYDYVGTGIIRSSNQSL
AGGITYVSNSGNLLGFKNVSTGNIFIVTPCNQPDQVAVYQQSIIGAMTAVNESRYGLQNLLQLPNFYYVSNG
GNNCTTAVMTYSNFGICADGSLIPVGPGNSSDNGISAIITANLSIPSNWTTSVQVEYLQITSTPIVVDCATY
VCNGNPRCKNLLKQYTSACKTIEDALRLSAHLETNDVSSMLTEDSNAFSLANVTSFGDYNLSSVLPQRNIGS
SGIAGSSPLEDLLESKVVTSGLGTVDVDYKSCTKGLSIADLACAQYYNGIMVLPGVADAERMAMYTGSLIGG
MVLGGLISAPAIPFSLALQARLNYVALQTDVLQENQKILAASENKAINNIVASFSSVNDPITQTAEAIHTVT
IALNKIQDVVNQQGSALNHLTSQLRHNFQAISNSIQAIYDRLDPPQADQQVDRLITGRLAALNAFVSQVLNK
YTEVRSSRRLAQQKVNECVKSQSNRYGFCGNGTHIFSIVNSAPDGLLFLHTVLLPTDYKNVKAWSGICVDGI
YGYVLRQPNLVLYSDNGVERVTSRIMFQPRLPVLSDFVQIYNCNVTFVNISRVELHTVIPDYVDVNKTLQEF
AQNLPKYVKPNFDLTPFNLTYLNLSSELKQLEAKTASLFQTTVELQGLIDQINSTYVDLKLLNRFENYIKWP
>OC43 (GenBank: AIX10763.1),
SEQ ID NO: 332
MFLILLISLPTAFAVIGDLNCPLDPKLKGSENNRDTGSPSISTDTVDVINGLGTYYVLDRVYLNTTLFLNGY
YPTSGSTYRNMALKGTDLLSTLWFKPPFLSDFINGIFAKVKNTKVFKDGVMYSEFPAITIGSTEVNTSYSVV
VQPRTINSTQDGVNKLQGLLEVSVCQYNMCEHPHTICHPNLGNHFKELWHLDTGVVSCLYKRNFTYDVNATY
LYFHFYQEGGTFYAYFTDTGFVTKFLFNVYLGMALSHYYVMPLTCISRRDIGFTLEYWVTPLTPRQYLLAEN
QDGIIFNAVDCMSDFMSEIKCKTQSIAPPTGVYELNGYTVQPIADVYRRKPDLPNCNIEAWLNDKSVPSPLN
WERKTFSNCNFNMSSLMSFIQADSFTCNNIDAAKIYGMCFSSITIDKFAIPNRRKVDLQLGNLGYLQSSNYR
IDTTATSCQLYYNLPAANVSVSRENPSTWNKRFGFIEDSVFVPQPTGVFTNHSVVYAQHCFKAPKNFCPCSS
CPGKNNGIGTCPAGTNSLTCDNLCTLDPITFKAPDTYKCPQTKSLVGIGEHCSGLAVKSDYCGNNSCTCQPQ
AFLGWSADSCLQGDKCNIFANFILHDVNNGLTCSTDLQKANTEIELGVCVNYDLYGISGQGIFVEVNATYYN
SWQNLLYDSNGNLYGFRDYITNRTFMIHSCYSGRVSAAYHANSPEPALLFRNIKCNYVENNSLTRQLQPINY
SFDSYLGCVVNAYNSTAISVQTCDLTVGSGYCVDYSKNSGSGSAITTGYRFTNFEPFTVNSVNDSLEPVGGL
YEIQIPSEFTIGNMEEFIQTSSPKVTIDCAAFVCGDYAACKLQLVEYGSFCDNINAILTEVNELLDTTQLQV
ANSLMNGVTLSTKLKDGVNFNVDDINFSPVLGCLGSECSKPSSASAIEDLLEDKVKLSDVGFVEAYNNCIGG
AEIRDLICVQSYKGIKVLPPLLSENQISGYTLAATSASLFPPWTAPAGVPFYLNVQYRINGLGVTMDVLSQN
QKLIANAFNNALHAIQQGFDPTNSALVKIQAVVNANSEALNNLLQQLSNRFGAISASLQEILSRLDPPEAEA
QIDRLINGRLTALNAYVSQQLSDSTLVKFSAAQAMEKVNECVKSQSSRINFCGNGNHIISLVQNAPYGLYFI
HFNYVPTKYVTAKVSPGLCIAGNRGIAPKSGYFVNVNNTWMYTGSGYYYPEPITENNVVVMSTCAVNYTKAP
YVMLNTSIPNLPDFKEELGQWFKNQTSVAPDLSLDYINVTELDLQVEMNRLQEAIKVLNHSYINLKDIGTYE
YYVKWP
>HKU1 (GenBank: AGW27881.1),
SEQ ID NO: 333
MLLIIFILPTTLAVIGDENCINFAINDKNTTVPRISEYVVDVSYGLGTYYILDRVYLNTTILFTGYFPKSGA
NERDLSLKGTTYLSTLWYQKPFLSDENNGIFSRVKNTKLYVNKTLYSEFSTIVIGSVFINNSYTIVVQPHNG
VLEITACQYTMCEYPHTICKSKGSSRNESWHEDKSEPLCLFKKNFTYNVSTDFLYFHFYQERGIFYAYYADS
GMPTTFLFSLYLGTLLSHYYVLPLICNAISSNIDNETLQYWVTPLSKRQYLLKFDNRGVITNAVDCSSSFFS
EIQCKTKSLLPNTGVYDLSGFTVKPVATVHRRIPDLPDCDIDKWLNNENVPSPLNWERKIFSNCNENLSTLL
RLVHTDSFSCNNFDESKIYGSCFKSIVLDKFAIPNSRRSDLQLGSSGFLQSSNYKIDTTSSSCQLYYSLPAI
NVTINNYNPSSWNRRYGENNENLSSHSVVYSRYCFSVNNTFCPCAKPSFASSCKSHKPPSASCPIGTNYRSC
ESTTVLDHTDWCRCSCLPDPITAYDPRSCSQKKSLVGVGEHCAGFGVDEEKCGVLDGSYNVSCLCSTDAFLG
WSYDTCVSNNRCNIFSNFILNGINSGTTCSNDLLQPNTEVYTDVCVDYDLYGITGQGIFKEVSAVYYNSWQN
LLYDSNGNIIGFKDFVINKTYNIFPCYAGRVSAAFHQNASSLALLYRNLKCSYVLNNISLATQPYFDSYLGC
VENADNLTDYSVSSCALRMGSGFCVDYNSPSFSSSGSKGSSISASYRFVTFEPFNVSFVNDSIESVGGLYEI
KIPTNFTIVGQEEFIQTNSPKVTIDCSLFVCSNYAACHDLLSEYGTFCDNINSILDEVNGLLDITQLHVADT
LMQGVTLSSNLNTNLHFDVDNINFKSLVGCLGPHCGSPSASFFEDLLEDKVKLSDVGFVEAYNNCTGGSEIR
DLLCVQSFNGIKVLPPILSESQISGYTTAATVAAMFPPWSAPAGIPFSLNVQYRINGLGVTMDVLNKNQKLI
ATAFNNALLSIQNGFSPINSALAKIQSVVNSNAQALNSLLQQLENKEGAISSSLQEILSRLDPPEAQVQIDR
LINGRLIALNAYVSQQLSDISLVKFGAALAMEKVNECVKSQSPRINFCGNGNHILSLVQNAPYGLLFMHFSY
KPISFKTVLVSPGLCISGDVGIAPKQGYFIKHNDHWMFTGSSYYYPEPISDKNVVEMNTCSVNETKAPLVYL
NHSVPKLSDFESELSHWFKNQTSIAPNLILNLHTINATELDLYYEMNLIQESIKSLNNSYINLKDIGTYEMY
VKWP

In some aspects, the recombinant SARS-CoV-1 S ectodomain included in the fusion protein comprises the “6P” mutations and an S1/S2 cleavage site mutation and an amino acid sequence at least 90% (such as at least 95%, at least 96%, at least 97%, at least 98%, or at least 99%) identical to residues 14-1188 of SEQ ID NO: 322 or 328. In some aspects, the recombinant SARS-CoV-1 S ectodomain included in the fusion protein comprises or consists of residues 14-1188 of SEQ ID NO: 322 or 328.

In some aspects, the recombinant MERS-CoV S ectodomain included in the fusion protein comprises the “6P” mutations and an S1/S2 cleavage site mutation and an amino acid sequence at least 90% (such as at least 95%, at least 96%, at least 97%, at least 98%, or at least 99%) identical to residues 18-1294 of SEQ ID NO: 323 or 329. In some aspects, the recombinant MERS-CoV S ectodomain included in the fusion protein comprises or consists of residues 18-1291 of SEQ ID NO: 323 or 329.

In some aspects, the recombinant 229E S ectodomain included in the fusion protein comprises the “5P” mutations and S1/S2 as well as S2 cleavage site mutations and an amino acid sequence at least 90% (such as at least 95%, at least 96%, at least 97%, at least 98%, or at least 99%) identical to residues 17-1112 of SEQ ID NO: 324 or 330. In some aspects, the recombinant 229E S ectodomain included in the fusion protein comprises or consists of residues 17-1112 of SEQ ID NO: 324 or 330.

In some aspects, the recombinant NL63 S ectodomain included in the fusion protein comprises the “5P” mutations and S1/S2 as well as S2 cleavage site mutations and an amino acid sequence at least 90% (such as at least 95%, at least 96%, at least 97%, at least 98%, or at least 99%) identical to residues 16-1296 of SEQ ID NO: 325 or 331. In some aspects, the recombinant NL63 S ectodomain included in the fusion protein comprises or consists of residues 16-1296 of SEQ ID NO: 325 or 331.

In some aspects, the recombinant OC43 S ectodomain included in the fusion protein comprises the “5P” mutations and S1/S2 as well as S2 cleavage site mutations and an amino acid sequence at least 90% (such as at least 95%, at least 96%, at least 97%, at least 98%, or at least 99%) identical to residues 15-1302 of SEQ ID NO: 326 or 332. In some aspects, the recombinant OC43 S ectodomain included in the fusion protein comprises or consists of residues 15-1302 of SEQ ID NO: 326 or 332.

In some aspects, the recombinant HKU1 S ectodomain included in the fusion protein comprises the “5P” mutations and S1/S2 as well as S2 cleavage site mutations and an amino acid sequence at least 90% (such as at least 95%, at least 96%, at least 97%, at least 98%, or at least 99%) identical to residues 14-1300 of SEQ ID NO: 327 or 333. In some aspects, the recombinant HKU1 S ectodomain included in the fusion protein comprises or consists of residues 14-1300 of SEQ ID NO: 327 or 333.

The recombinant SARS-CoV-1, MERS-CoV, 229E, NL63, OC43, or HKU1S ectodomain included in the fusion protein is fused to HBsAg via a peptide linker. Any suitable peptide linker may be used that allows for folding and trimerization of the SARS-CoV-1, MERS-CoV, 229E, NL63, OC43, or HKU1 S ectodomain on the surface of the HBsAg nanoparticle, for example, peptide linkers as described above for the SARS-CoV-2 S ectodomain.

Exemplary SARS-CoV-1, MERS-CoV, 229E, NL63, OC43, or HKU1 S fusion protein sequences encoded by the nucleic acid molecules are provided herein, including those listed inn Table 2:

TABLE 2
Exemplary SARS-CoV-1, MERS-CoV, 229E, NL63, OC43,
and HKU1 S ectodomain - HBsAg fusion proteins.

| SEQ ID | |
|---|---|
| NO | Sequence description |
| | S-8-HBsAg S sequences, with Spike ectodomain based on: |
| 297 | SARS-CoV-1 |
| 298 | MERS-CoV |
| 334 | 229E (Based on GenBank: ABB90529.1) |
| 335 | NL63 (Based on GenBank: APF29063.1) |
| 336 | OC43 (Based on GenBank: AIX10763.1) |
| 337 | HKU1 (Based on GenBank: AGW27881.1) |
| | S-12-HBsAg S sequences, with Spike ectodomain based on: |
| 299 | SARS-CoV-1 |
| 300 | MERS-CoV |
| 338 | 229E S5P (Based on GenBank: ABB90529.1) |
| 339 | NL63 (Based on GenBank: APF29063.1) |
| 340 | OC43 (Based on GenBank: AIX10763.1) |
| 341 | HKU1 (Based on GenBank: AGW27881.1) |
| | S-16-HBsAg S sequences, with Spike ectodomain based on: |
| 301 | SARS-CoV-1 |
| 302 | MERS-CoV |
| 342 | 229E S5P (Based on GenBank: ABB90529.1) |
| 343 | NL63 (Based on GenBank: APF29063.1) |
| 344 | OC43 (Based on GenBank: AIX10763.1) |
| 345 | HKU1 (Based on GenBank: AGW27881.1) |
| | S-8-HBsAg M sequences, with Spike ectodomain based on: |
| 303 | SARS-CoV-1 |
| 304 | MERS-CoV |
| 346 | 229E S5P (Based on GenBank: ABB90529.1) |
| 347 | NL63 (Based on GenBank: APF29063.1) |
| 348 | OC43 (Based on GenBank: AIX10763.1) |
| 349 | HKU1 (Based on GenBank: AGW27881.1) |
| | S-12-HBsAg M sequences, with Spike ectodomain based on: |
| 305 | SARS-CoV-1 |
| 306 | MERS-CoV |
| 350 | 229E S5P (Based on GenBank: ABB90529.1) |
| 351 | NL63 (Based on GenBank: APF29063.1) |
| 352 | OC43 (Based on GenBank: AIX10763.1) |
| 353 | HKU1 (Based on GenBank: AGW27881.1) |
| | S-16-HBsAg M sequences, with Spike ectodomain based on: |
| 307 | SARS-CoV-1 |
| 308 | MERS-CoV |
| 354 | 229E S5P (Based on GenBank: ABB90529.1) |
| 355 | NL63 (Based on GenBank: APF29063.1) |
| 356 | OC43 (Based on GenBank: AIX10763.1) |
| 357 | HKU1 (Based on GenBank: AGW27881.1) |
| | S-8-HBsAg L sequences, with Spike ectodomain based on: |
| 309 | SARS-CoV-1 |
| 310 | MERS-CoV |
| 358 | 229E S5P (Based on GenBank: ABB90529.1) |
| 359 | NL63 (Based on GenBank: APF29063.1) |
| 360 | OC43 (Based on GenBank: AIX10763.1) |
| 361 | HKU1 (Based on GenBank: AGW27881.1) |
| | S-12-HBsAg L sequences, with Spike ectodomain based on: |
| 311 | SARS-CoV-1 S6P |
| 312 | MERS-CoV S |
| 362 | 229E S5P (Based on GenBank: ABB90529.1) |
| 363 | NL63 (Based on GenBank: APF29063.1) |
| 364 | OC43 (Based on GenBank: AIX10763.1) |
| 365 | HKU1 (Based on GenBank: AGW27881.1) |
| | S-16-HBsAg L sequences, with Spike ectodomain based on: |
| 313 | SARS-CoV-1 |
| 314 | MERS-CoV |
| 366 | 229E S5P (Based on GenBank: ABB90529.1) |
| 367 | NL63 (Based on GenBank: APF29063.1) |
| 368 | OC43 (Based on GenBank: AIX10763.1) |
| 369 | HKU1 (Based on GenBank: AGW27881.1) |

The SARS-CoV-1 fusion protein sequences provided in Table 2 include a signal peptide (residues 1-13) that is not present in the protein as expressed in mammalian cells. In some aspects, the fusion protein encoded by the nucleic acid molecule comprises or consists of an amino acid sequence at least 90% (such as at least 95%, at least 96%, at least 97%, at least 98%, at least 99%) identical to residues 14 to the C-terminus of any one of the SARS-CoV-1-HBsAg fusion proteins provided in Table 2, such as any one of SEQ DD NOs: 297, 299, 301, 303, 305, 307, 309, 311, or 313. In some aspects, the fusion protein encoded by the nucleic acid molecule comprises or consists of the amino acid sequence set forth as residues 14 to the C-terminus of any one of the SARS-CoV-1 HBsAg fusion proteins provided in Table 2, such as any one of SEQ ID NOs: 297, 299, 301, 303, 305, 307, 309, 311, or 313.

The MERS-CoV fusion protein sequences provided in Table 2 include a signal peptide (residues 1-17) that is not present in the protein as expressed in mammalian cells. In some aspects, the fusion protein encoded by the nucleic acid molecule comprises or consists of an amino acid sequence at least 90% (such as at least 95%, at least 96%, at least 97%, at least 98%, at least 99%) identical to residues 18 to the C-terminus of any one of the MERS-CoV—HBsAg fusion proteins provided in Table 2, such as any one of SEQ ID NOs: 298, 300, 302, 304, 306, 308, 310, 312, or 314. In some aspects, the fusion protein encoded by the nucleic acid molecule comprises or consists of the amino acid sequence set forth as residues 14 to the C-terminus of any one of the SARS-CoV-1 HBsAg fusion proteins provided in Table 2, such as any one of SEQ ID NOs: 298, 300, 302, 304, 306, 308, 310, 312, or 314.

The 229E-CoV fusion protein sequences provided in Table 2 include a signal peptide (residues 1-16) that is not present in the protein as expressed in mammalian cells. In some aspects, the fusion protein encoded by the nucleic acid molecule comprises or consists of an amino acid sequence at least 90% (such as at least 95%, at least 96%, at least 97%, at least 98%, at least 99%) identical to residues 17 to the C-terminus of any one of the MERS-CoV—HBsAg fusion proteins provided in Table 2, such as any one of SEQ ID NOs: 334, 338, 342, 346, 350, 354, 358, 362, or 366. In some aspects, the fusion protein encoded by the nucleic acid molecule comprises or consists of the amino acid sequence set forth as residues 17 to the C-terminus of any one of the SARS-CoV-1 HBsAg fusion proteins provided in Table 2, such as any one of SEQ ID NOs: 334, 338, 342, 346, 350, 354, 358, 362, or 366.

The NL63-CoV fusion protein sequences provided in Table 2 include a signal peptide (residues 1-15) that is not present in the protein as expressed in mammalian cells. In some aspects, the fusion protein encoded by the nucleic acid molecule comprises or consists of an amino acid sequence at least 90% (such as at least 95%, at least 96%, at least 97%, at least 98%, at least 99%) identical to residues 16 to the C-terminus of any one of the MERS-CoV—HBsAg fusion proteins provided in Table 2, such as any one of SEQ ID NOs: 335, 339, 343, 347, 351, 355, 359, 363, or 367. In some aspects, the fusion protein encoded by the nucleic acid molecule comprises or consists of the amino acid sequence set forth as residues 16 to the C-terminus of any one of the SARS-CoV-1 HBsAg fusion proteins provided in Table 2, such as any one of SEQ ID NOs: 335, 339, 343, 347, 351, 355, 359, 363, or 367.

The OC43-CoV fusion protein sequences provided in Table 2 include a signal peptide (residues 1-14) that is not present in the protein as expressed in mammalian cells. In some aspects, the fusion protein encoded by the nucleic acid molecule comprises or consists of an amino acid sequence at least 90% (such as at least 95%, at least 96%, at least 97%, at least 98%, at least 99%) identical to residues 15 to the C-terminus of any one of the MERS-CoV-HBsAg fusion proteins provided in Table 2, such as any one of SEQ ID NOs: 336, 340, 344, 348, 352, 356, 360, 364, or 368. In some aspects, the fusion protein encoded by the nucleic acid molecule comprises or consists of the amino acid sequence set forth as residues 15 to the C-terminus of any one of the SARS-CoV-1 HBsAg fusion proteins provided in Table 2, such as any one of SEQ ID NOs: 336, 340, 344, 348, 352, 356, 360, 364, or 368.

The HKU1-CoV fusion protein sequences provided in Table 2 include a signal peptide (residues 1-13) that is not present in the protein as expressed in mammalian cells. In some aspects, the fusion protein encoded by the nucleic acid molecule comprises or consists of an amino acid sequence at least 90% (such as at least 95%, at least 96%, at least 97%, at least 98%, at least 99%) identical to residues 14 to the C-terminus of any one of the MERS-CoV-HBsAg fusion proteins provided in Table 2, such as any one of SEQ ID NOs: 337, 341, 345, 349, 353, 357, 361, 365, or 369. In some aspects, the fusion protein encoded by the nucleic acid molecule comprises or consists of the amino acid sequence set forth as residues 14 to the C-terminus of any one of the SARS-CoV-1 HBsAg fusion proteins provided in Table 2, such as any one of SEQ ID NOs: 337, 341, 345, 349, 353, 357, 361, 365, or 369.

Additional aspects of the disclosure are provided with the following clauses:

Clause 1. A nucleic acid molecule encoding a fusion protein comprising:
a recombinant Coronavirus spike(S) ectodomain comprising 817P, 892P, 899P, 942P, 986P, and 987P substitutions, or 817P, 899P, 942P, 986P, and 987P substitutions according to the reference Coronavirus S protein set forth as SEQ ID NO: 1, and fused via a peptide linker to a hepatitis B surface antigen (HBsAg) protein.

Clause 2. The nucleic acid molecule of Clause 1, wherein the fusion protein self-assembles when expressed in mammalian cells to form a HBsAg nanoparticle with Coronavirus S ectodomain trimers extending radially outward from an outer surface of the HBsAg nanoparticle.

Clause 3. The nucleic acid molecule of Clause 1 or Clause 2, wherein a S1/S2 protease cleavage site of the Coronavirus S ectodomain is mutated to inhibit protease cleavage.

Clause 4. The nucleic acid molecule of any one of the prior Clauses, wherein the Coronavirus S protein ectodomain is a SARS-CoV-1, MERS-CoV, 229E, NL63, OC43, or HKU1 S ectodomain.

Clause 5. The nucleic acid molecule of any one of the prior Clauses, wherein the Coronavirus S ectodomain comprises the 817P, 892P, 899P, 942P, 986P, and 987P substitutions, or the 817P, 899P, 942P, 986P, and 987P substitutions according to the reference Coronavirus S protein set forth as SEQ ID NO: 1;
the S1/S2 protease cleavage site of the Coronavirus S ectodomain is mutated to inhibit protease cleavage;
and the amino acid sequence of the Coronavirus S ectodomain is at least 90% identical, at least 95% identical, at least 99% identical, or identical, to:
residues 14-1188 of SEQ ID NO: 322 or 328;
residues 18-1291 of SEQ ID NO: 323 or 329;
residues 17-1112 of SEQ ID NO: 324 or 330;
residues 16-1296 of SEQ ID NO: 325 or 331;
residues 15-1302 of SEQ ID NO: 326 or 332; or
residues 14-1300 of SEQ ID NO: 327 or 333.

Clause 6. The nucleic acid molecule of any one of the prior Clauses, wherein the peptide linker is from 12-39 amino acid residues in length.

Clause 7. The nucleic acid molecule of Clause 6, wherein the peptide linker is from 12-32 residues in length.

Clause 8. The nucleic acid molecule of Clause 6, wherein the peptide linker is 24 or 32 residues in length.

Clause 9. The nucleic acid molecule of any one of the prior Clauses, wherein the peptide linker is a glycine-serine linker.

Clause 10. The nucleic acid molecule of Clause 9, wherein the glycine-serine peptide linker is a repeat of glycine-serine dipeptides.

Clause 11. The nucleic acid molecule of any one of the prior Clauses, wherein the sequence of the peptide linker is set forth as any one of SEQ ID NOs: 4-7.

Clause 12. The nucleic acid molecule of any one of the prior Clauses, wherein the HBsAg protein comprises or consists of a HBsAg S protein, a HBsAg M protein, or a HBsAg L protein.

Clause 13. The nucleic acid molecule of Clause 12, wherein the HBsAg protein comprises or consists of the HBsAg S protein.

Clause 14. The nucleic acid molecule of Clauses 12 or 13, wherein the HBsAg protein comprises or consists of an amino acid sequence at least 90% identical, at least 95% identical, at least 99% identical, or identical, to any one of SEQ ID NOs: 9 or 45-56.

Clause 15. The nucleic acid molecule of Clause 15 or Clause 16, wherein the HBsAg protein comprises or consists of an amino acid sequence at least 90% identical, at least 95% identical, at least 99% identical, or identical, to SEQ ID NO: 9.

Clause 16. The nucleic acid molecule of any one of the prior Clauses, wherein the fusion protein comprises or consists of an amino acid sequence at least 90% identical, at least 95% identical, at least 99% identical, or identical, to
residues 14 to the C-terminus of any one SEQ ID NOs: 297, 299, 301, 303, 305, 307, 309, 311, or 313;
residues 14 to the C-terminus of any one of SEQ ID NOs: 298, 300, 302, 304, 306, 308, 310, 312, or 314;
residues 17 to the C-terminus of any one of SEQ ID NOs: 334, 338, 342, 346, 350, 354, 358, 362, or 366;
residues 16 to the C-terminus of any one of SEQ ID NOs: 335, 339, 343, 347, 351, 355, 359, 363, or 367;
residues 15 to the C-terminus of any one of SEQ ID NOs: 336, 340, 344, 348, 352, 356, 360, 364, or 368; or
residues 14 to the C-terminus of any one of SEQ ID NOs: 337, 341, 345, 349, 353, 357, 361, 365, or 369.

Clause 17. The nucleic acid molecule of any one of the prior Clauses, wherein the nucleic acid molecule is DNA or RNA.

Clause 18. The nucleic acid molecule of any one of the prior Clauses, wherein the nucleic acid molecule is an mRNA or circular RNA.

Clause 19. A vector comprising the nucleic acid molecule of any one of the prior Clauses.

Clause 20. The vector of Clause 19, wherein the vector is a DNA vector, an RNA vector, or a viral vector.

Clause 21. A HBsAg protein nanoparticle encoded by the nucleic acid or vector of any one of the prior Clauses.

Clause 22. The HBsAg protein nanoparticle of Clause 21, comprising Coronavirus S ectodomain trimers extending radially outward from an outer surface of the nanoparticle.

Clause 23. An immunogenic composition comprising the nucleic acid molecule, vector, or HBsAg protein nanoparticle of any one of the prior Clauses and a pharmaceutically acceptable carrier.

Clause 24. The composition of Clause 23, wherein the nucleic acid molecule is an mRNA or circular RNA and the composition further comprises a lipid nanoparticle.

Clause 25. A method, comprising administering to a subject an amount of the nucleic acid molecule, the vector, HBsAg protein nanoparticle, or the composition of any one of the prior Clauses in an amount effective to elicit a neutralizing immune response to Coronavirus in the subject.

Clause 26. The method of Clause 25, wherein the subject was previously exposed to HBsAg by hepatitis B infection or vaccination.

Clause 27. The method of Clause 25 or Clause 26, wherein the immune response inhibits or prevents infection by the Coronavirus in the subject.

Clause 28. The method of any one of Clauses 25-27, wherein the immune response inhibits or prevents severe disease due to the Coronavirus in the subject.

### C. Additional Description

Analogs and variants of the recombinant Coronavirus S ectodomain (e.g., SARS-CoV-2, SARS-CoV-1, MERS-CoV, 229E, NL63, OC43, or HKU1 S ectodomain), peptide linker, or HBsAg included in the disclosed fusion protein may be used in the methods and systems of the present disclosure, as long as the resulting fusion protein self-assembles to form a HBsAg nanoparticle with Coronavirus S ectodomain (e.g., SARS-CoV-2, SARS-CoV-1, MERS-CoV, 229E, NL63, OC43, or HKU1 S ectodomain) trimers extending radially outward from an outer surface of the HBsAg nanoparticle, and wherein the Coronavirus S ectodomain (e.g., SARS-CoV-2, SARS-CoV-1, MERS-CoV, 229E, NL63, OC43, or HKU1 S ectodomain) trimer maintains binding to cell surface receptor. Through the use of recombinant nucleic acid technology, variants of the recombinant Coronavirus S ectodomain (e.g., SARS-CoV-2, SARS-CoV-1, MERS-CoV, 229E, NL63, OC43, or HKU1 S ectodomain), peptide linker, or HBsAg included in the disclosed fusion protein may be prepared by altering the encoding nucleic acid sequence. Non-limiting examples of such variants include insertions, substitutions, or deletions of one or more amino acid residues to further stabilize the prefusion conformation of the S ectodomain, to add mutations from variant Coronavirus strains, or to add or remove glycan sequon.

The nucleic acid molecule encoding the fusion protein can be any suitable type of nucleic acid molecule including DNA (such as cDNA) and RNA (such as mRNA, circular RNA), as well as modified forms thereof (such as but are not limited to modified mRNA with N1-methylpseudouridine in place of uridine), that encode the fusion protein, as well as vectors including the DNA, cDNA and RNA sequences, such as a DNA or RNA vector used for immunization. The genetic code may be used to construct a variety of functionally equivalent nucleic acids, such as nucleic acids which differ in sequence but which encode the same fusion protein sequence.

Exemplary nucleic acids can be prepared by cloning techniques. Examples of appropriate cloning and sequencing techniques, and instructions sufficient to direct persons of skill through many cloning exercises are known (see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013).

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

The polynucleotides can include a recombinant DNA which is incorporated into a vector (such as an expression vector) into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

Polynucleotide sequences can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

Nucleic acid molecules encoding the disclosed fusion proteins can be expressed in vitro by transfer into a suitable host cell. The cell may be prokaryotic or eukaryotic. The term also includes any progeny of the subject host cell. It is understood that all progenies may not be identical to the parental cell since there may be mutations that occur during replication. Methods of stable transfer, meaning that the foreign DNA is continuously maintained in the host, are known in the art.

### IV. Immunogenic Compositions

Immunogenic compositions comprising a disclosed nucleic acid molecule encoding a Coronavirus S ectodomain-HBsAg fusion protein (such as a SARS-CoV-2 S ectodomain-HBsAg fusion protein), or a HBsAg nanoparticle displaying the Coronavirus S ectodomain (such as SARS-CoV-2 S ectodomain) encoded by the nucleic acid molecule, and a pharmaceutically acceptable carrier are also provided. Such compositions can be administered to subjects by any suitable method, for example, intramuscular, intradermal, subcutaneous, intravenous, intra-arterial, intra-articular, intraperitoneal, intranasal, sublingual, tonsillar, oropharyngeal, or other parenteral and mucosal routes.

Thus, a nucleic acid molecule encoding a Coronavirus S ectodomain-HBsAg fusion protein (such as a SARS-CoV-2 S ectodomain-HBsAg fusion protein), or a HBsAg nanoparticle displaying the Coronavirus S ectodomain (such as SARS-CoV-2 S ectodomain) encoded by the nucleic acid molecule, as described herein can be formulated with pharmaceutically acceptable carriers to help retain biological activity while also promoting increased stability during storage within an acceptable temperature range. Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The immunogenic composition may optionally include an adjuvant to enhance an immune response of the host. Suitable adjuvants are, for example, toll-like receptor agonists, alum, AlPO4, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), among many other suitable adjuvants well known in the art, may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product.

In some instances it may be desirable to combine a disclosed immunogen with other pharmaceutical products (e.g., vaccines) which induce protective responses to other agents. For example, a composition including a nucleic acid molecule encoding a Coronavirus S ectodomain-HBsAg fusion protein (such as a SARS-CoV-2 S ectodomain-HBsAg fusion protein), or a HBsAg nanoparticle displaying the Coronavirus S ectodomain (such as SARS-CoV-2 S ectodomain) by the nucleic acid molecule, as described herein can be can be administered simultaneously (typically separately) or sequentially with other vaccines recommended by the Advisory Committee on Immunization Practices (ACIP; cdc.gov/vaccines/acip/index.html) for the targeted age group (e.g., infants from approximately one to six months of age), such as an influenza vaccine or a varicella zoster vaccine. As such, a disclosed immunogen including a nucleic acid molecule encoding a SARS-CoV-2 S ectodomain-HBsAg fusion protein described herein may be administered simultaneously or sequentially with vaccines against, for example, hepatitis B (HepB), diphtheria, tetanus and pertussis (DTaP), pneumococcal bacteria (PCV), Haemophilus influenzae type b (Hib), polio, influenza and rotavirus.

In some aspects, the composition can be provided as a sterile composition. Typically, the amount of immunogen in each dose of the immunogenic composition is selected as an amount which induces an immune response without significant, adverse side effects. In some aspects, the composition can be provided in unit dosage form for use to induce an immune response in a subject. A unit dosage form contains a suitable single preselected dosage for administration to a subject, or suitable marked or measured multiples of two or more preselected unit dosages, and/or a metering mechanism for administering the unit dose or multiples thereof. In other aspects, the composition further includes an adjuvant.

The immunogenic compositions provided herein can be formulated for mucosal vaccination, such as intranasal administration. Mucosal vaccination can be achieved by a number of routes including oral, intranasal, pulmonary, rectal and vaginal. In a specific example, this is achieved by intranasal administration. Thus, in some examples the disclosed compositions are formulated for intranasal administration.

For example, the disclosed compositions can include one or more biodegradable, mucoadhesive polymeric carriers. Polymers such as polylactide-co-glycolide (PLGA), chitosan, alginate and carbopol can be included. Hydrophilic polymers, like sodium alginate and carbopol, absorb to the mucus by forming hydrogen bonds, consequently enhancing nasal residence time, and thus can be included in the disclosed compositions.

In one example, the composition includes sodium alginate, which is a linear copolymer and consists of 1-4-linked β-d-mannuronic acid and 1-4-linked α-1-guluronic acid residues. In some examples, the composition includes alginate microspheres. In one example, the composition includes carbopol (a cross-linked polyacrylic acid polymer), for example in combination with starch. In some examples, the composition includes chitosan, a non-toxic linear polysaccharide that can be produced by chitin deacetylation. In one example the chitosan is in the form of chitosan nanoparticles, such as N-trimethyl chitosan (TMC)-based nanoparticles.

In one example, the composition is formulated as a particulate delivery system used for nasal administration. In one example the composition can include liposomes, immune-stimulating complexes (ISCOMs) and/or polymeric particles, such as virosomes. In one example, the liposome is surface-modified (e.g., glycol chitosan or oligomannose coated). In one example, the liposome is fusogenic or cationic-fusogenic.

The compositions can also include one or more lipopeptides of bacterial origin, or their synthetic derivatives. Examples of lipid moieties include tri-palmitoyl-S-glyceryl cysteine (Pam3Cys), di-palmitoyl-S-glyceryl cysteine (Pam2Cys), single/multiple-chain palmitic acids and lipoamino acids (LAAs).

The compositions can also include one or more adjuvants, for example a mucosal adjuvant, such as one or more of CpG oligodeoxynucleotides (CpG ODN), Flt3 ligand, and monophosphoryl lipid A (MLA). In one example, the adjuvant includes a clinical grade MLA formulation, such as MPL® (3-O-desacyl-4′-monophosphoryl lipid A) adjuvant.

### V. Methods of Eliciting an Immune Response

The disclosed nucleic acid molecule encoding a Coronavirus S ectodomain-HBsAg fusion protein (such as a SARS-CoV-2 S ectodomain-HBsAg fusion protein), or a HBsAg nanoparticle displaying the Coronavirus S ectodomain (such as SARS-CoV-2 S ectodomain) encoded by the nucleic acid molecule, can be administered to a subject to induce an immune response to the Coronavirus S protein in the subject. In a particular example, the subject is a human. Following administration of the nucleic acid molecule, the Coronavirus S ectodomain-HBsAg fusion protein (such as a SARS-CoV-2 S ectodomain-HBsAg fusion protein) is produced within cells of the subject, and self-assembles to form HBsAg nanoparticles with Coronavirus S ectodomain (such as SARS-CoV-2 S ectodomain) trimers extending radially outward from the outer surface of the HBsAg nanoparticle. The HBsAg nanoparticle displaying the Coronavirus S ectodomain trimers (such as SARS-CoV-2 S ectodomain trimers), whether administered as protein or encoding nucleic acid molecule, elicits the desired immune response to the Coronavirus S ectodomain (such as a SARS-CoV-2 S ectodomain) in the subject. The immune response can be a protective immune response, for example a response that inhibits subsequent infection with the Coronavirus (such as SARS-CoV-2). Elicitation of the immune response can also be used to treat or inhibit Coronavirus infection (such as SARS-CoV-2 infection) and illnesses associated with the infection.

A subject can be selected for treatment that has or is at risk for developing a Coronavirus (such as SARS-CoV-2) infection, for example because of exposure or the possibility of exposure to the Coronavirus. Following administration of a disclosed immunogen, the subject can be monitored for infection or symptoms associated with Coronavirus infection.

Typical subjects intended for vaccination with the nucleic acid molecule encoding a Coronavirus S ectodomain—HBsAg fusion protein (such as a SARS-CoV-2 S ectodomain-HBsAg fusion protein), or a HBsAg nanoparticle displaying the Coronavirus S ectodomain (such as SARS-CoV-2 S ectodomain) encoded by the nucleic acid molecule, of the present disclosure include humans, as well as non-human primates and other animals. To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize coronavirus infection. These and other routine methods allow the clinician to select patients in need of therapy using the methods and pharmaceutical compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

The administration of a disclosed nucleic acid molecule encoding a Coronavirus S ectodomain-HBsAg fusion protein (such as a SARS-CoV-2 S ectodomain-HBsAg fusion protein), or a HBsAg nanoparticle displaying the Coronavirus S ectodomain (such as SARS-CoV-2 S ectodomain) encoded by the nucleic acid molecule, can be for prophylactic or therapeutic purpose. When provided prophylactically, the immunogen is provided in advance of infection. The prophylactic administration serves to prevent or ameliorate any subsequent infection. When provided therapeutically, the immunogen is provided at or after the onset of a symptom of infection, for example, after development of a symptom of SARS-CoV-2 infection or after diagnosis with the SARS-CoV-2 infection. The nucleic acid molecule encoding a Coronavirus S ectodomain—HBsAg fusion protein (such as a SARS-CoV-2 S ectodomain—HBsAg fusion protein), or a HBsAg nanoparticle displaying the Coronavirus S ectodomain (such as SARS-CoV-2 S ectodomain) encoded by the nucleic acid molecule, can thus be provided prior to the anticipated exposure to the Coronavirus so as to attenuate the anticipated severity, duration or extent of an infection and/or associated disease symptoms, after exposure or suspected exposure to the Coronavirus, or after the actual initiation of an infection.

The nucleic acid molecule encoding a Coronavirus S ectodomain—HBsAg fusion protein (such as a SARS-CoV-2 S ectodomain—HBsAg fusion protein), or a HBsAg nanoparticle displaying the Coronavirus S ectodomain (such as SARS-CoV-2 S ectodomain) encoded by the nucleic acid molecule, and compositions thereof, are provided to a subject in an amount effective to induce or enhance an immune response against the Coronavirus S protein in the subject, preferably a human. The actual dosage of disclosed immunogen may vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

A composition including the nucleic acid molecule encoding a Coronavirus S ectodomain—HBsAg fusion protein (such as a SARS-CoV-2 S ectodomain—HBsAg fusion protein), or a HBsAg nanoparticle displaying the Coronavirus S ectodomain (such as SARS-CoV-2 S ectodomain) encoded by the nucleic acid molecule, can be used in coordinate (or prime-boost) vaccination protocols or combinatorial formulations. There can be several boosts. In some examples the boost may be the same nucleic acid molecule encoding a SARS-CoV-2 S ectodomain—HBsAg fusion protein or HBsAg nanoparticle displaying the SARS-CoV-2 ectodomain encoded by the nucleic acid molecule as another boost, or the prime. The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. For example a relatively large dose in a primary immunization and then a boost with relatively smaller doses.

In some aspects, the boost can be administered about two, about three to eight, or about four, weeks following the prime, or about several months after the prime. In some aspects, the boost can be administered about 5, about 6, about 7, about 8, about 10, about 12, about 18, about 24, months after the prime, or more or less time after the prime. Periodic additional boosts can also be used at appropriate time points to enhance the subject's “immune memory.” The adequacy of the vaccination parameters chosen, e.g., formulation, dose, regimen and the like, can be determined by taking aliquots of serum from the subject and assaying antibody titers during the course of the immunization program. In addition, the clinical condition of the subject can be monitored for the desired effect, e.g., prevention of infection or improvement in disease state (e.g., reduction in viral load). If such monitoring indicates that vaccination is sub-optimal, the subject can be boosted with an additional dose of immunogenic composition, and the vaccination parameters can be modified in a fashion expected to potentiate the immune response.

The amount of nucleic acid molecule encoding a Coronavirus S ectodomain—HBsAg fusion protein (such as a SARS-CoV-2 S ectodomain—HBsAg fusion protein), or a HBsAg nanoparticle displaying the Coronavirus S ectodomain (such as SARS-CoV-2 S ectodomain) encoded by the nucleic acid molecule, utilized in an immunogenic composition may be selected based on the subject population (e.g., infant or elderly). An optimal amount for a particular composition can be ascertained by standard studies involving observation of antibody titers and other responses in subjects. It is understood that an effective amount of a disclosed nucleic acid molecule encoding a Coronavirus S ectodomain—HBsAg fusion protein (such as a SARS-CoV-2 S ectodomain—HBsAg fusion protein), or a HBsAg nanoparticle displaying the Coronavirus S ectodomain (such as SARS-CoV-2 S ectodomain) encoded by the nucleic acid molecule, can include an amount that is ineffective at eliciting an immune response by administration of a single dose, but that is effective upon administration of multiple dosages, for example in a prime-boost administration protocol.

In some aspects, the antibody response of a subject will be determined in the context of evaluating effective dosages/immunization protocols. In most instances it will be sufficient to assess the antibody titer in serum or plasma obtained from the subject. Decisions as to whether to administer booster inoculations and/or to change the amount of the nucleic acid molecule encoding a Coronavirus S ectodomain—HBsAg fusion protein (such as a SARS-CoV-2 S ectodomain—HBsAg fusion protein), or a HBsAg nanoparticle displaying the Coronavirus S ectodomain (such as SARS-CoV-2 S ectodomain) encoded by the nucleic acid molecule, administered to the individual can be at least partially based on the antibody titer level. The antibody titer level can be based on, for example, an immunobinding assay which measures the concentration of antibodies in the serum which bind to an antigen including, for example, the recombinant SARS-CoV-2 S ectodomain included in the fusion protein.

The Coronavirus infection does not need to be completely eliminated or reduced or prevented for the methods to be effective. For example, elicitation of an immune response to Coronavirus with one or more of the disclosed nucleic acid molecule encoding a Coronavirus S ectodomain—HBsAg fusion protein, or a HBsAg nanoparticle displaying the Coronavirus ectodomain encoded by the nucleic acid molecule, can reduce or inhibit infection with the Coronavirus by a desired amount, for example, by at least 10%, at least 20%, at least 50%, at least 60%, at least 70%, at least 80%, at least 90%, at least 95%, at least 98%, or even at least 100% (elimination or prevention of detectable infected cells), as compared to infection in the absence of the immunogen. In additional examples, replication of the Coronavirus can be reduced or inhibited by the disclosed methods. Replication does not need to be completely eliminated for the method to be effective. For example, the immune response elicited using one or more of the disclosed immunogens can reduce replication of the Coronavirus by a desired amount, for example, by at least 10%, at least 20%, at least 50%, at least 60%, at least 70%, at least 80%, at least 90%, at least 95%, at least 98%, or even at least 100% (elimination or prevention of detectable replication), as compared to replication in the absence of the immunogen.

SARS-CoV-2 infection does not need to be completely eliminated or reduced or prevented for the methods to be effective. For example, elicitation of an immune response to SARS-CoV-2 with one or more of the disclosed nucleic acid molecule encoding a SARS-CoV-2 S ectodomain—HBsAg fusion protein, or a HBsAg nanoparticle displaying the SARS-CoV-2 ectodomain encoded by the nucleic acid molecule, can reduce or inhibit SARS-CoV-2 infection by a desired amount, for example, by at least 10%, at least 20%, at least 50%, at least 60%, at least 70%, at least 80%, at least 90%, at least 95%, at least 98%, or even at least 100% (elimination or prevention of detectable infected cells), as compared to SARS-CoV-2 infection in the absence of the immunogen. In additional examples, SARS-CoV-2 replication can be reduced or inhibited by the disclosed methods. SARS-CoV-2 replication does not need to be completely eliminated for the method to be effective. For example, the immune response elicited using one or more of the disclosed immunogens can reduce SARS-CoV-2 replication by a desired amount, for example, by at least 10%, at least 20%, at least 50%, at least 60%, at least 70%, at least 80%, at least 90%, at least 95%, at least 98%, or even at least 100% (elimination or prevention of detectable SARS-CoV-2 replication), as compared to SARS-CoV-2 replication in the absence of the immunogen.

In some aspects, the disclosed nucleic acid molecule encoding a Coronavirus S ectodomain-HBsAg fusion protein (such as a SARS-CoV-2 S ectodomain—HBsAg fusion protein), or a HBsAg nanoparticle displaying the Coronavirus S ectodomain (such as SARS-CoV-2 S ectodomain) encoded by the nucleic acid molecule, is administered to the subject simultaneously with the administration of the adjuvant. In other aspects, the disclosed immunogen is administered to the subject after the administration of the adjuvant and within a sufficient amount of time to induce the immune response.

One approach to administration of nucleic acids is direct immunization with plasmid DNA, such as with a mammalian expression plasmid. Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. Nos. 5,593,972 and 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil ATM (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In some aspects, a plasmid DNA vaccine is used to express a disclosed immunogen in a subject. For example, a nucleic acid molecule encoding a disclosed immunogen can be administered to a subject to induce an immune response to the Coronavirus S protein (such as SARS-CoV-2 S protein) included in the immunogen. In some aspects, the nucleic acid molecule can be included on a plasmid vector for DNA immunization, such as the pVRC8400 vector (described in Barouch et al., J. Virol, 79, 8828-8834, 2005, which is incorporated by reference herein). Additional non-limiting vector examples include CMVR8400 or pVRC8400, CMV8X, pcDNA™3.1 (+) 3.2, 4, 5, 6.2 series of vectors, pCAGGS, pCAGGS-G-Kan, pBOOST (InvivoGen), and pVAC (InvivoGen).

In another approach to using nucleic acids for immunization, the nucleic acid molecule can be expressed by attenuated viral hosts or vectors or bacterial vectors. Recombinant vaccinia virus, adeno-associated virus (AAV), herpes virus, retrovirus, cytomegalo virus or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In one aspect, a nucleic acid molecule may be introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

In another aspect, an mRNA-based immunization protocol can be used to deliver a nucleic acid encoding the Coronavirus S ectodomain—HBsAg fusion protein (such as a SARS-CoV-2 S ectodomain-HBsAg fusion protein) to elicit an immune response to the Coronavirus. mRNA vaccines preclude safety concerns about DNA integration into the host genome and can be directly translated in the host cell cytoplasm. Moreover, cell-free, in vitro synthesis of RNA avoids the manufacturing complications associated with viral vectors.

In some aspects, mRNA vaccination is achieved using mRNA encoding a Coronavirus S ectodomain—HBsAg fusion protein as described herein and formulated as a lipid nanoparticle according to known methods, such as those described in WO2021154763, US20210228707, WO2017070626 and US2019/0192646, which are incorporated by reference herein. See, also, Jackson et al., N Engl J Med., 383 (20): 1920-1921, 2020, incorporated by reference herein. For example the mRNA component is a modified mRNA with 1-methylpseudouridine in place of uridine and a 7 mG (5′) ppp (5′) N1mpNp cap. The mRNA sequence includes a 5′ untranslated region (UTR), the immunogen (Coronavirus S ectodomain—HBsAg fusion protein open reading frame), a 3′ UTR, and a polyA tail. In some aspects, the ORF sequence is codon optimized relative to native sequence for mRNA expression in a human and to increase stability. In several aspects, the mRNA is formulated in a lipid nanoparticle; for example, comprising a PEG-modified lipid, a non-cationic lipid, a sterol, an ionizable lipid, or any combination thereof. In some aspects, the lipid nanoparticle is composed of 50 mol % ionizable lipid ((2 hydroxyethyl) (6 oxo 6-(undecycloxy) hexyl)amino) octanoate, 10 mol % 1,2 distearoyl sn glycerol-3 phosphocholine (DSPC), 38.5 mol % cholesterol, and 1.5 mol % 1-monomethoxypolyethyleneglycol-2,3, dimyristylglycerol with polyethylene glycol of average molecular weight 2000 (PEG2000 DMG). The mRNA/lipid nanoparticle composition may be provided in any suitable carrier, such as a sterile liquid for injection at a concentration of 0.5 mg/mL in 20 mM trometamol (Tris) buffer containing 87 mg/mL sucrose and 10.7 mM sodium acetate, at pH 7.5 and with appropriate diluent.

Additional exemplary forms of RNA-based vaccination that can be used to deliver a nucleic acid encoding a Coronavirus S ectodomain—HBsAg fusion protein as described herein include conventional non-amplifying mRNA immunization (see, e.g., Petsch et al., “Protective efficacy of in vitro synthesized, specific mRNA vaccines against influenza A virus infection,” Nature biotechnology, 30 (12): 1210-6, 2012) and self-amplifying mRNA immunization (see, e.g., Geall et al., “Nonviral delivery of self-amplifying RNA vaccines,” PNAS, 109 (36): 14604-14609, 2012; Magini et al., “Self-Amplifying mRNA Vaccines Expressing Multiple Conserved Influenza Antigens Confer Protection against Homologous and Heterosubtypic Viral Challenge,” PLOS One, 11 (8): e0161193, 2016; and Brito et al., “Self-amplifying mRNA vaccines,” Adv Genet., 89:179-233, 2015). In another aspect, a circular RNA (circRNA)-based immunization protocol can be used to deliver a nucleic acid encoding the Coronavirus S ectodomain-HBsAg fusion protein (such as a SARS-CoV-2 S ectodomain—HBsAg fusion protein) to elicit an immune response to the Coronavirus. In contrast to linear RNA, circRNA is stable due to its covalently closed ring structure, which protects it from exonuclease-mediated degradation. Although circRNA lacks the essential elements for cap-dependent translation, it can be engineered to enable protein translation through internal ribosome entry site (IRES) or the m6A modification incorporated to its 5′ UTR region. (See, e.g., Wesselhoeft, P. S. Kowalski, D. G. Anderson, Engineering circular RNA for potent and stable translation in eukaryotic cells. Nat Commun. 9, 2629, 2018; Yang et al., Extensive translation of circular RNAs driven by N(6)-methyladenosine. Cell Res. 27, 626-641, 2017; Kristensen et al. The biogenesis, biology, and characterization or circular RNAs. Nat. Rev. Genetics, 20, 675-691, 2029).

In some aspects, administration of a therapeutically effective amount of one or more of the disclosed immunogens to a subject induces a neutralizing antibody response in the subject. To assess neutralization activity, following immunization of a subject, serum can be collected from the subject at appropriate time points, frozen, and stored for neutralization testing. Methods to assay for neutralization activity include, but are not limited to, plaque reduction neutralization (PRNT) assays, microneutralization assays, flow cytometry based assays, single-cycle infection assays. In some aspects, the serum neutralization activity can be assayed using a SARS-CoV-2 pseudovirus, for example, as described in Corbett et al., Nature 586, 567-571, 2020, which is incorporated by reference herein in its entirety.

### EXAMPLES

The following examples are provided to illustrate certain particular features and/or aspects. These examples should not be construed to limit the disclosure to the particular features or aspects described.

### Example 1

### Self-Assembling SARS-CoV-2 Spike—HBsAg Nanoparticles Elicit Potent and Durable Neutralizing Antibody Responses via Genetic Delivery

While several COVID-19 vaccines have been in use, more effective and durable vaccines are needed to combat the ongoing COVID-19 pandemic. In this example, we report highly immunogenic self-assembling SARS-CoV-2 spike-HBsAg nanoparticles displaying a six-proline-stabilized WA1 spike S6P on a HBsAg core. These S6P—HBsAgs bound diverse domain-specific SARS-CoV-2 monoclonal antibodies. In mice with and without a HBV pre-vaccination, nucleic acid immunization with S6P—HBsAgs elicited significantly more potent and durable neutralizing antibody (nAb) responses against diverse SARS-CoV-2 strains than that of soluble S2P or S6P, or full-length S2P with its coding sequence matching mRNA-1273. The nAb responses elicited by S6P—HBsAgs can persist 7 months longer than by soluble S2P or S6P and appeared to be enhanced by HBsAg pre-exposure. These data show that genetic delivery of SARS-CoV-2 S6P—HBsAg nanoparticles can elicit greater and more durable nAb responses than non-nanoparticle forms of stabilized spike.

### RESULTS

### SARS-CoV-2 S6P—HBsAg Constructs can Express Self-Assembling Nanoparticles

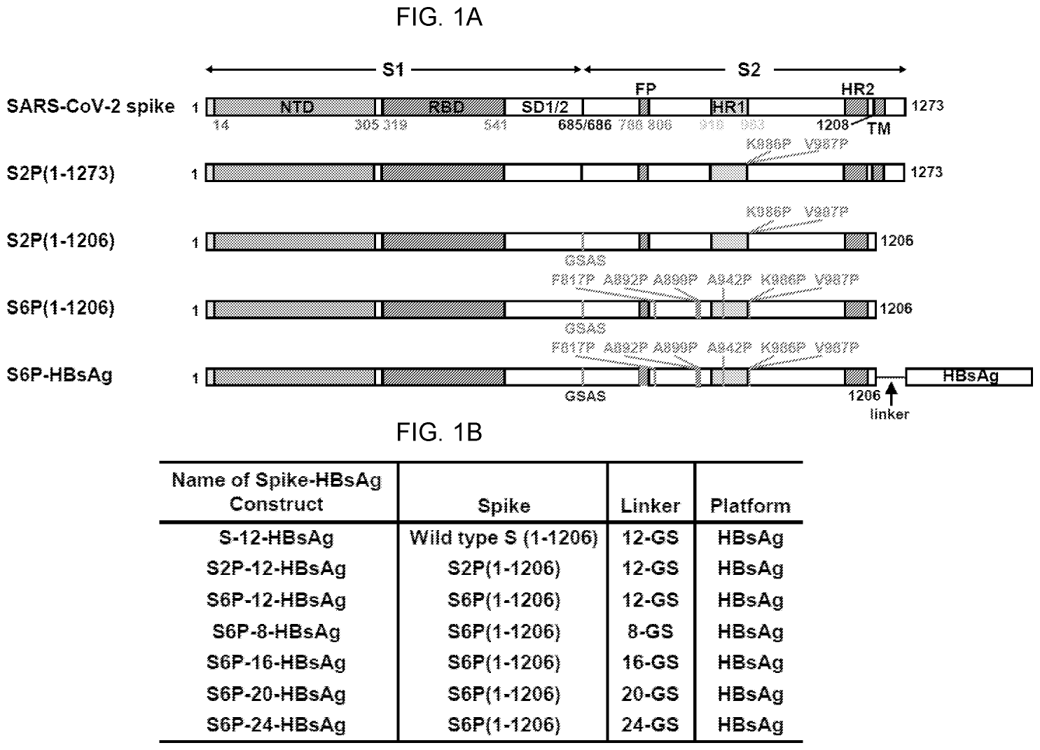

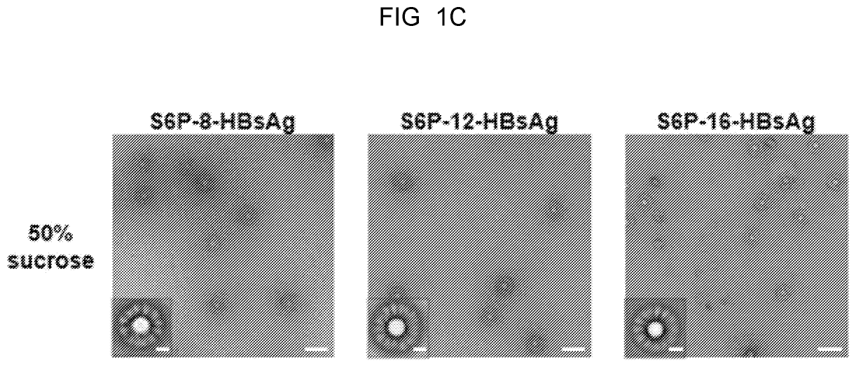

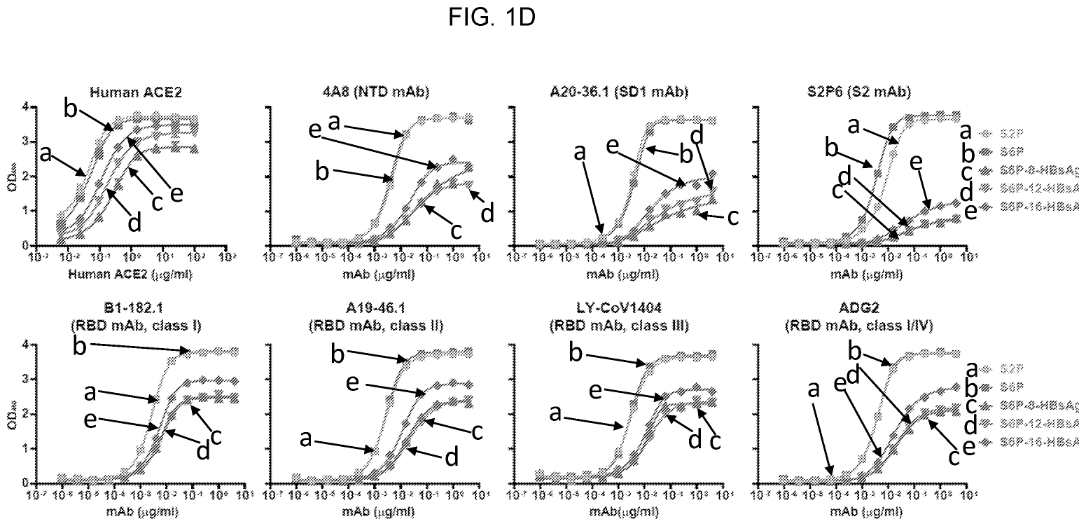

We designed plasmid DNAs encoding fusion proteins of SARS-CoV-2 WA1 spike, S2P and S6P, which consist of the corresponding ectodomain (amino acids (aa) 1-1206) fused to HBsAg (aa 1-226) by GS linkers of varying lengths (FIGS. 1A, 1B). After transfection into Expi293 cells, these constructs expressed proteins that can be detected in the 30% to 65% sucrose fractions after ultracentrifugation using a sucrose gradient. These fractions from all tested constructs showed binding to anti-HBsAg antibody and four mAbs specific to SARS-CoV-2 spike: NTD mAb S652-118, RBD mAbs A23-58.1 and LY-CoV555, and S2 mAb WS6 (FIG. 6, shown only for S6P—HBsAgs). Small fractions of WA1 spike and S2P formed well-defined nanoparticles. S6P-8-HBsAg from the 50% sucrose faction, and S6P-12-HBsAg and S6P-16-HBsAg from 50% and 65% sucrose fractions formed well-defined nanoparticles (FIG. 1C). These nanoparticles had diameters of ˜70 nm, with a yield of 12 to 16 mg/L for each construct. In these HBsAg nanoparticles, the S6P ectodomain in its prefusion conformation was displayed on the surface. Image analysis quantifying the numbers of spike trimers suggested up to 80 trimers per nanoparticle.

SARS-CoV-2 S6P—HBsAg Nanoparticles can Bind Human ACE2 and Diverse SARS-CoV-2 mAbs

To characterize these SARS-CoV-2 S6P—HBsAg nanoparticles, we tested the binding of purified S6P-8-HBsAg, S6P-12-HBsAg and S6P-16-HBsAg nanoparticles to human ACE2, as ACE2 is one of the primary receptors for SARS-CoV-2. WA1 S2P and S6P recombinant proteins were used as controls, as they adopt stabilized prefusion conformation (Wrapp et al., Science, 2020. 367 (6483): p. 4; Hsieh et al., Science, 2020. 369 (6510): p. 5). While S2P and S6P bound to human ACE2 with similar patterns (FIG. 1D), all three S6P—HBsAgs showed moderately weaker binding to human ACE2 than S2P and S6P. Among these three types of nanoparticles, S6P-8-HBsAg appeared to be the weakest binder and S6P-16-HBsAg was the strongest binder.

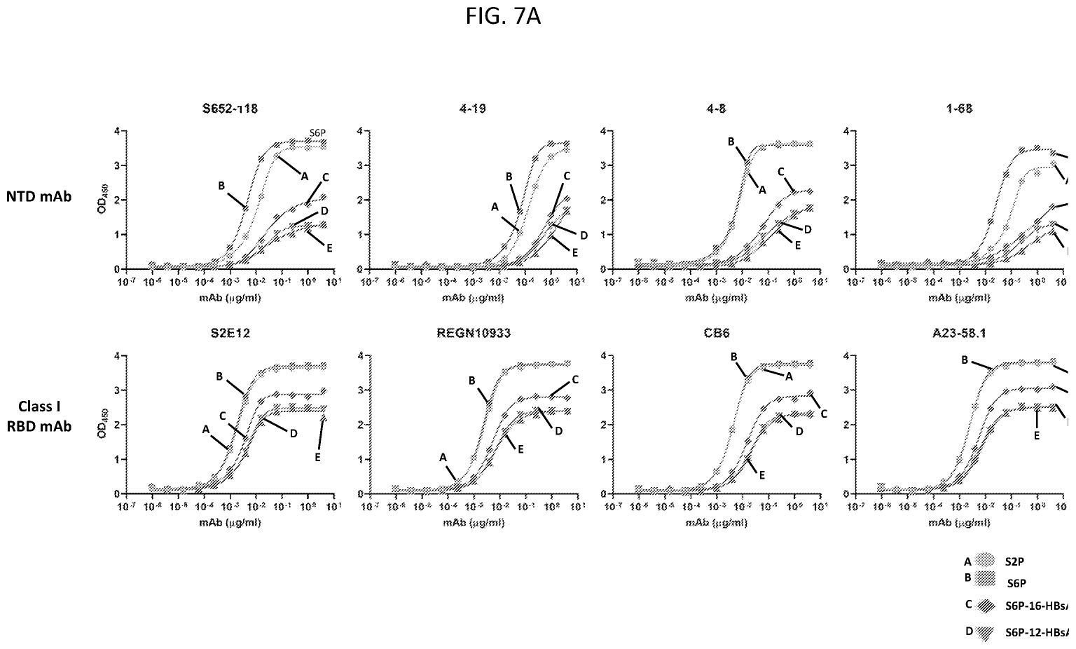

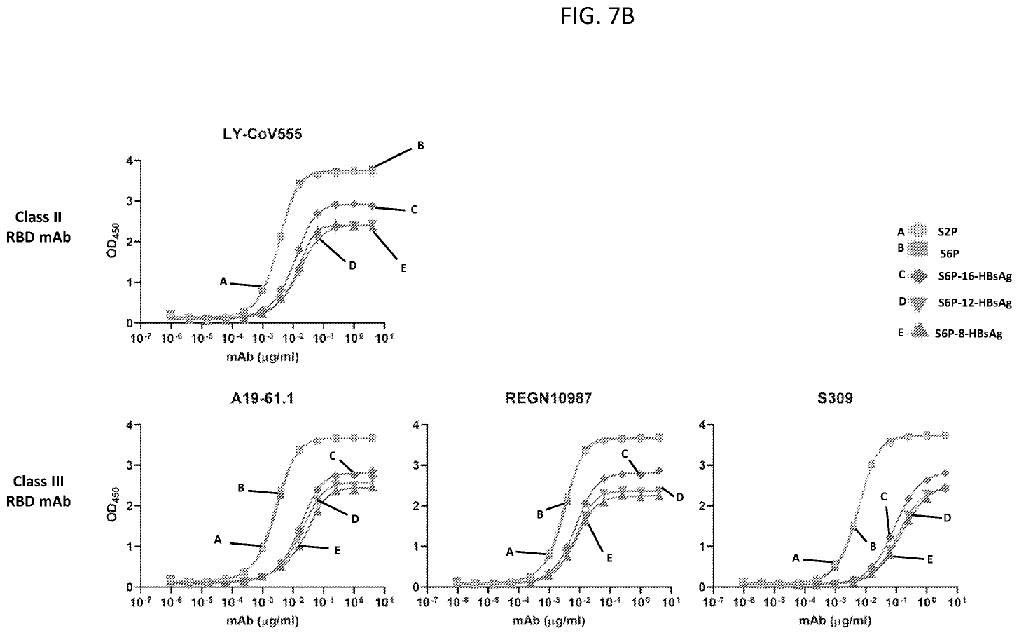

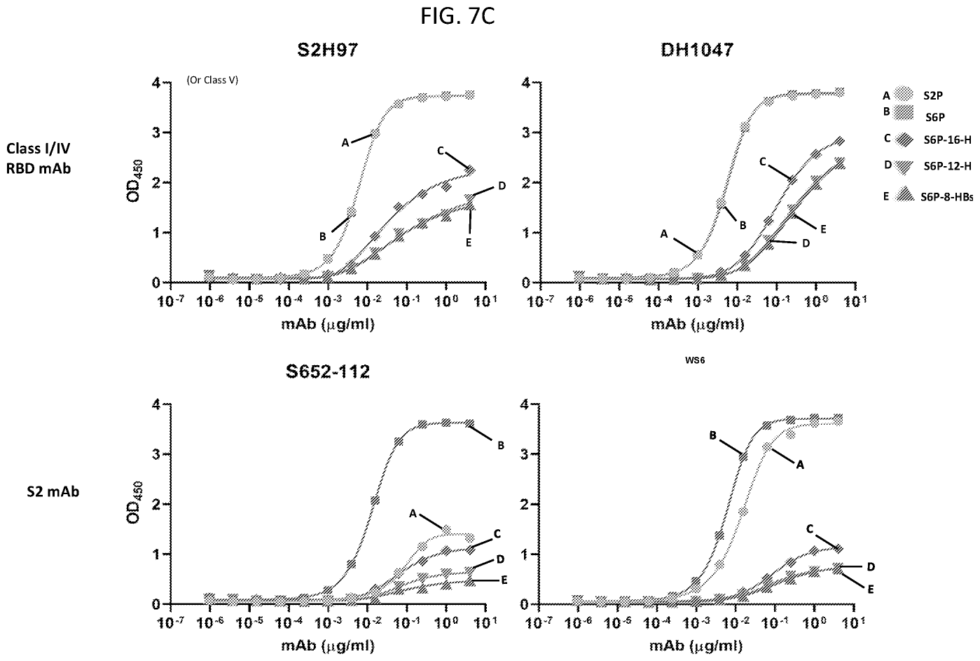

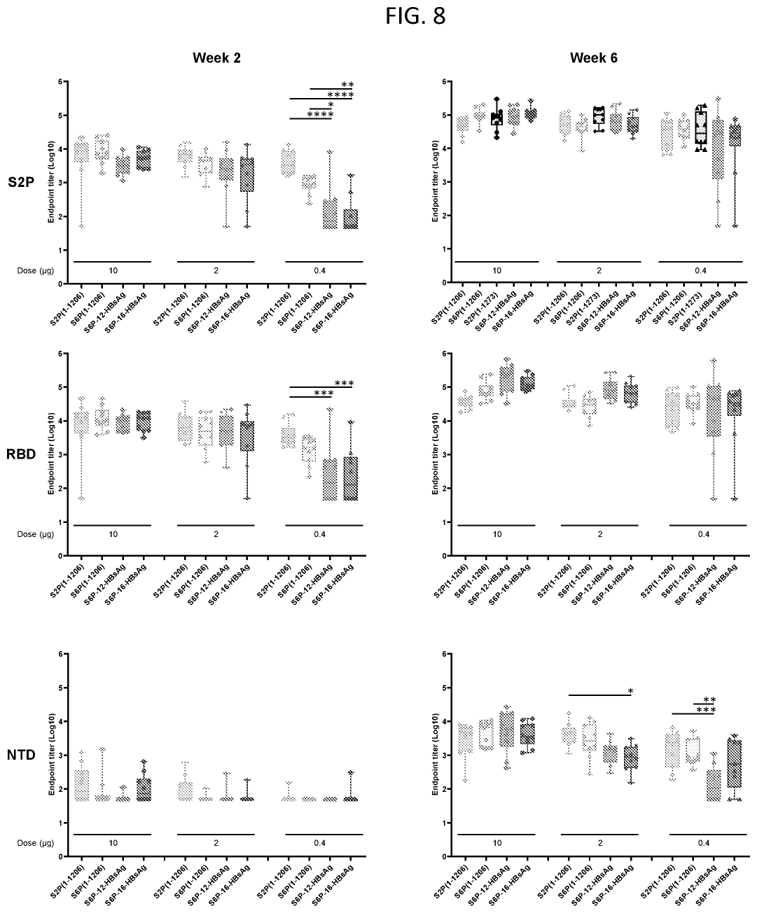

To monitor whether individual domains of SARS-CoV-2 spike within the nanoparticles are accessible, we evaluated the antigenicity of these three S6P—HBsAgs. mAbs specific to the NTD, SD1, S2 and RBD domains of SARS-CoV-2 spike were used for this evaluation (Table 3). S2P and S6P showed nearly identical binding profiles to NTD mAbs 4A8 and 4-8, SD1 mAb A20-36.1, and all tested RBD mAbs (FIGS. 1D, 7). S6P exhibited marginally higher affinities to NTD mAbs S652-118, 4-19 and 1-68, S2 mAbs S2P6 and WS-6, and remarkably higher affinity to S2 mAb S652-112 than S2P. Compared with S2P and S6P, the three S6P—HBsAgs showed weaker binding to all tested mAbs specific to the NTD, SD1, S2 and RBD domains of SARS-CoV-2 spike. While S6P-8-HBsAg and S6P-12-HBsAg showed similar binding affinities to most tested mAbs, S6P-8-HBsAg exhibited slightly lower affinities to NTD mAbs S652-118, 4-19 and 1-68, SD1 mAb A20-36.1, and S2 mAb S652-112. (The references for these mAbs are listed in Table 3). Among these three types of nanoparticles, S6P-16-HBsAg exhibited the highest affinities to all tested mAbs specific to the NTD, SD1, S2 and RBD domains of SARS-CoV-2 spike. Based on these antigenicity data, we chose S6P-12-HBsAg and S6P-16-HBsAg for immunogenicity studies.

TABLE 3
mAbs used in this study to evaluate the antigenicity
of SARS-CoV-2 S6P-HBsAg nanoparticles

| | Targeting | | |
|---|---|---|---|
| | domain of | | |
| | SARS-CoV-2 | Class of | |
| Antibody | spike | RBD mAbs | Reference |
| 4A8 | NTD | | Chi et al., Science, 2020. |
| | | | 369(6504): p. 6. |
| S652-118 | NTD | | Zhou et al., Cell Rep, 2020. |
| | | | 33(4). |
| 4-19 | NTD | | Liu et al., Nature, 2020. |
| | | | 584(7821): p. 7. |
| 4-8 | NTD | | Liu et al., Nature, 2020. |
| | | | 584(7821): p. 7. |
| 1-68 | NTD | | Liu et al., Nature, 2020. |
| | | | 584(7821): p. 7. |
| A20-36.1 | SD1 | | Corbett et al., Science, 2021. |
| | | | 374(6573): p. 11. |
| B1-182.1 | RBD up | Class I | Wang et al., Science, 2021. |
| | | | 373(6556). |
| S2E12 | RBD up | Class I | Tortorici et al., Science, 2020. |
| | | | 370(6519): p. 8. |
| REGN10933 | RBD up | Class I | Baum et al., Science, 2020. |
| | | | 370(6520): p. 16.; Drouin et al., |
| | | | Viruses, 2021. 13(7). |
| CB6 | RBD up | Class I | Shi et al., Nature, 2020. |
| | | | 584(7819): p. 5. |
| A23-58.1 | RBD up | Class I | Wang et al., Science, 2021. |
| | | | 373(6556). |
| A19-46.1 | RBD | Class II | Wang et al., Science, 2021. |
| | | | 373(6556); Zhou et al., |
| | | | Science, 2022. |
| LY-CoV555 | RBD | Class II | Jones et al., bioRxiv., 2020; |
| | | | Oct 9;2020.09.30.318972; |
| | | | Gottlieb et al., JAMA, 2021. |
| | | | 325(7): p. 13. |
| LY-CoV1404 | RBD | Class III | Westendorf et al., bioRxiv, 2022. |
| | | | 2021.04.30.442182. |
| A19-61.1 | RBD | Class III | Wang et al., Science, 2021. |
| | | | 373(6556). |
| REGN10987 | RBD | Class III | Baum et al., Science, 2020. |
| | | | 370(6520): p. 16.; Drouin et al., |
| | | | Viruses, 2021. 13(7). |
| S309 | RBD | Class III | Pinto et al., Nature, 2020. |
| | | | 583(7815): p. 6. |
| ADG2 | RBD | Class I/IV | Zhou et al., Science, 2022.; |
| | | | Rappazzo et al., Science, 2021. |
| | | | 371(6531): p. 7.; Liu et al., |
| | | | Nature, 2022. 602(7898): |
| | | | p. 6. |
| DH1047 | RBD | Class I/IV | Liu et al., Nature, 2022. |
| | | | 602(7898): p. 6.; |
| | | | Martinez et al., Sci Transl |
| | | | Med, 2022. 14(629). |
| S2H97 | RBD | Class V | Tortorici et al., Science, |
| | | | 2020. 370(6519): p. 8. |
| S2P6 | S2 | | Pinto et al., Science, 2021. |
| | | | 373(6559): p. 8. |
| S652-112 | S2 | | Zhou et al., Cell Rep, 2020. |
| | | | 33(4). |
| WS6 | S2 | | Shi et al., bioRxiv, 2022 Jan |
| | | | 26; 2022.01.25.477770. |

### DNA Encoding SARS-CoV-2 S6P—HBsAgs Elicits Potent Binding and Neutralizing Antibody Responses Against WA1

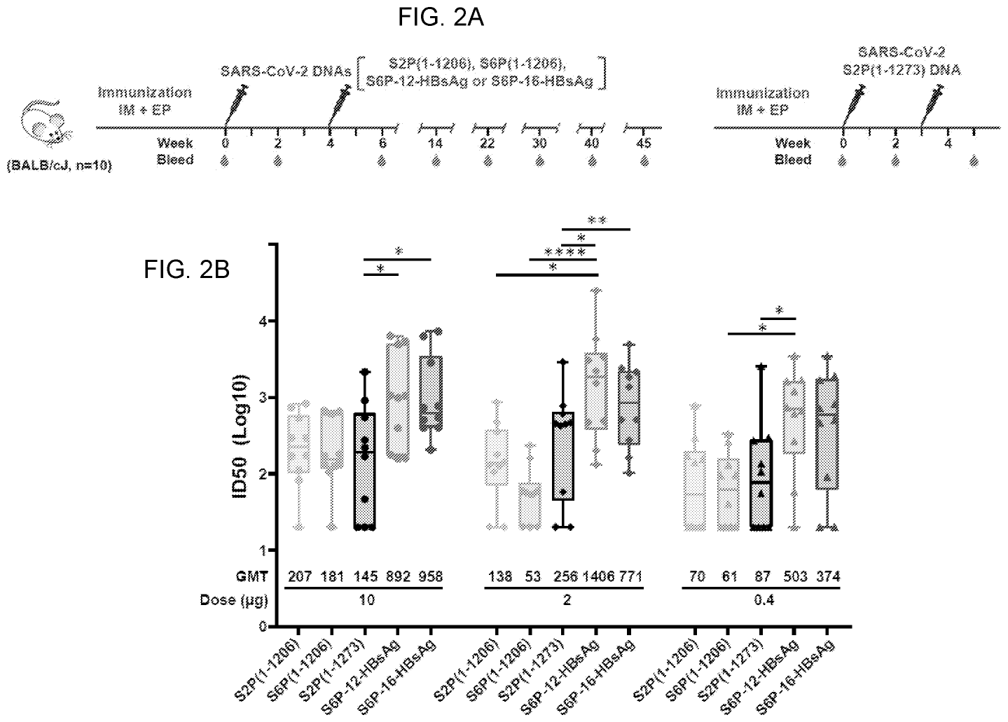

The immunogenicity of S6P-12-HBsAg and S6P-16-HBsAg was assessed in 6- to 8-week-old BALB/cJ mice (FIG. 2A). With two intramuscular immunizations plus electroporation spaced 4-week apart, 10, 2 and 0.4 μg of S6P-12-HBsAg or S6P-16-HBsAg elicited potent binding antibodies to WA1 S2P, RBD and NTD domains (FIG. 8). The geometric mean titers (GMTs) increased from week 2 to week 6. The GMTs against S2P or RBD elicited by S6P—HBsAgs were comparable to those elicited by the same dose of non-nanoparticle forms of stabilized spikes S2P (1-1206), S6P (1-1206) and S2P (1-1273) at week 6. 0.4 μg of S6P—HBsAg elicited lower GMTs than the same dose of soluble S2P and S6P. Due to the limited volume of sera available from mice immunized with S2P (1-1273), we did not perform ELISA binding to RBD or NTD for these sera.

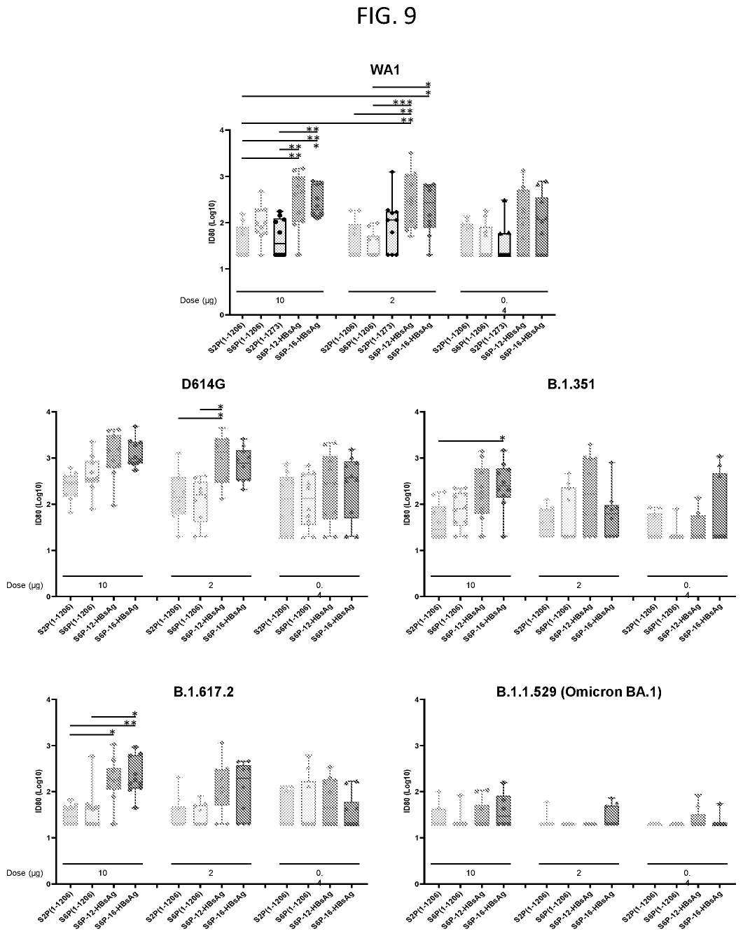

The neutralization potency elicited by SARS-CoV-2 S6P—HBsAg nanoparticles was tested using lentiviral-based WA1 pseudovirus in sera collected at 2 weeks post the second immunization. The same dose of S2P (1-1206), S6P (1-1206) and S2P (1-1273) matching the sequence of mRNA-1273 elicited comparable ID50s (FIG. 2B). Ten, 2 and 0.4 μg S6P-12-HBsAg or S6P-16-HBsAg DNA elicited 3- to 26.5-fold higher ID50s than the same dose of these three non-nanoparticle forms of stabilized spikes (Table 4). Two and 0.4 μg S6P-12-HBsAg or S6P-16-HBsAg DNA elicited significantly higher geometric mean ID50s than those elicited by the same or higher dose of non-nanoparticle forms of stabilized spikes (FIG. 2B, Table 5). Similar results were obtained for ID80s against WA1 pseudovirus (FIG. 9).

TABLE 4
Geometric mean neutralization ID50 titers against
WA1 pseudovirus at week 6 (to FIG. 2B)

| | S2P | S6P | S2P | S6P-12- | S6P-16- |
|---|---|---|---|---|---|
| Immunogen | (1-1206) | (1-1206) | (1-1273) | HBsAg | HBsAg |
| Dose | 10 μg | | | | |
| GMT | 207 | 181 | 145 | 892 | 958 |
| Fold of | 1.0 | 0.9 | 0.7 | 4.3 | 4.6 |
| Change | 1.1 | 1.0 | 0.8 | 4.9 | 5.3 |
| in GMT | 1.4 | 1.2 | 1.0 | 6.2 | 6.6 |
| Overall Fold | | | | 4.3~6.2 | 4.6~6.6 |
| of Change | | | | | |
| in GMT | | | | | |
| Dose | 2 μg | | | | |
| GMT | 138 | 53 | 256 | 1406 | 771 |
| Fold of | 1.0 | 0.4 | 1.9 | 10.2 | 5.6 |
| Change | 2.6 | 1.0 | 4.8 | 26.5 | 14.5 |
| in GMT | 0.5 | 0.2 | 1.0 | 5.5 | 3.0 |
| Overall Fold | | | | 5.5~26.5 | 3~14.5 |
| of Change | | | | | |
| in GMT | | | | | |
| Dose | 0.4 μg | | | | |
| GMT | 70 | 61 | 87 | 503 | 374 |
| Fold of | 1.0 | 0.9 | 1.2 | 7.2 | 5.3 |
| Change | 1.1 | 1.0 | 1.4 | 8.2 | 6.1 |
| in GMT | 0.8 | 0.7 | 1.0 | 5.8 | 4.3 |
| Overall Fold | | | | 5.8~8.2 | 4.3~6.1 |
| of Change | | | | | |
| in GMT | | | | | |

In Table 4, GMT: Geometric mean titer. Fold of change was calculated in comparison with the same dose of non-nanoparticle form of spike.

TABLE 5
Statistical analyses of geometric mean ID50s at
week 6 against WA1 pseudovirus (to FIG. 2B)

| Nanoparticle | Non-nanoparticle spike | p value |
|---|---|---|
| 2 μg S6P-12-HBsAg | 10 μg S6P(1-1206) | 0.0408 |
| | 2 μg S2P(1-1206) | 0.0103 |
| | 2 μg S6P(1-1206) | <0.0001 |
| 0.4 μg S6P-12-HBsAg | 2 μg S6P(1-1206) | 0.0144 |
| | 0.4 μg S6P(1-1206) | 0.0296 |
| 2 μg S6P-16-HBsAg | 2 μg S6P(1-1206) | 0.0012 |
| 10 μg S6P-12-HBsAg | 10 μg S2P(1-1273) | 0.043 |
| 2 μg S6P-12-HBsAg | 2 μg S2P(1-1273) | 0.0345 |
| 0.4 μg S6P-12-HBsAg | 0.4 μg S2P(1-1273) | 0.0341 |
| 10 μg S6P-16-HBsAg | 10 μg S2P(1-1273) | 0.0286 |

The statistical analyses shown in Table 5 were performed using the Two-way ANOVA test for the comparison between S6P—HBsAg and soluble S2P (1-1206), S6P (1-1206). The comparison between the same dose of S6P—HBsAg and S2P (1-1273) was done using two tailed Mann-Whitney test with Dunn's multiple comparisons test.

### DNA Encoding SARS-CoV-2 S6P—HBsAgs Elicits Potent Binding and Neutralizing Antibody Responses Against SARS-CoV-2 Variants

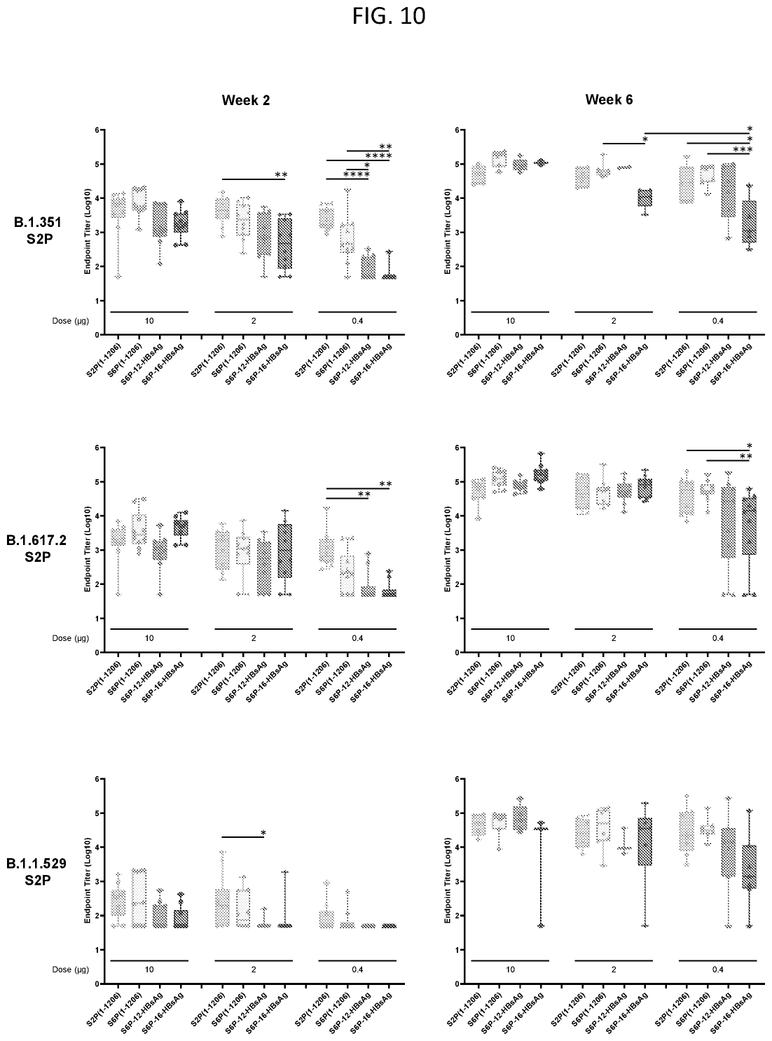

As SARS-CoV-2 variants D614G, B.1.351 (Beta), B.1.617.2 (Delta) and B.1.1.529 (Omicron BA.1) pose greater risk to public health than WA1, we also tested whether S6P—HBsAgs can elicit potent antibody responses against these variants. S6P-12-HBsAg and S6P-16-HBsAg elicited binding antibody responses to B.1.351, B.1.617.2 and B.1.1.529 S2Ps in a dose-dependent manner at both weeks 2 and 6 (FIG. 10). The GMTs increased from week 2 to week 6. At each time point, 0.4 μg S6P—HBsAgs elicited substantially lower GMTs against all three variant S2Ps than the same dose of soluble S2P or S6P.

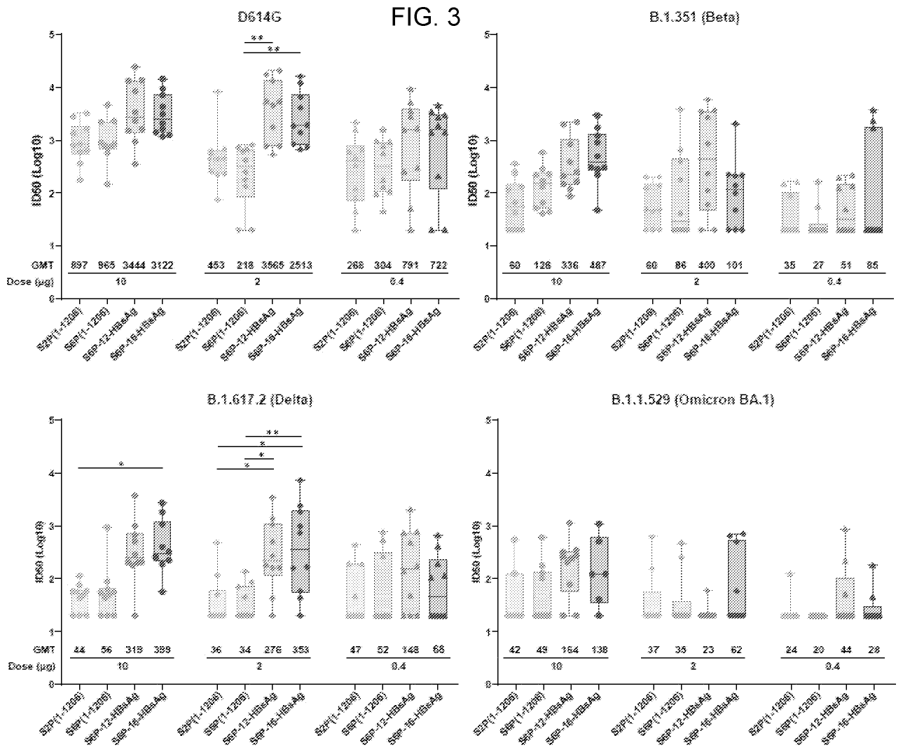

Ten, 2 and 0.4 μg of soluble S2P and S6P elicited similar levels of neutralization potency against each individual pseudovirus at each dose (FIG. 3, Table 6). At all three doses, S6P-12-HBsAg or S6P-16-HBsAg elicited substantially higher geometric mean ID50s against D614G, B.1.351 and B.1.617.2 pseudoviruses than the same dose of soluble S2P and S6P. Two μg of S6P-12-HBsAg and 10 or 2 μg S6P-16-HBsAg elicited significantly higher neutralization potency than the same or higher doses of soluble S2P or S6P for D614G and B.1.617.2 pseudoviruses (FIG. 3, Table 7). For B.1.1.529 pseudovirus, S6P—HBsAgs elicited marginally increased geometric mean ID50s compared with soluble S2P and S6P. Similar results were obtained for ID80 titers (FIG. 9).

TABLE 6
Geometric mean neutralization ID50 titers against SARS-
CoV-2 variant pseudovirus at week 6 (to FIG. 3)

| | S2P | S6P | S6P-12- | S6P-16- |
|---|---|---|---|---|
| Immunogen | (1-1273) | (1-1206) | HBsAg | HBsAg |

Dose 10 μg

| | | | | | |
|---|---|---|---|---|---|
| D614G | GMT | 550 | 876 | 5237 | 3376 |
| | Fold Change | 897 | 965 | 3444 | 3122 |
| | in GMT | 1.0 | 1.1 | 3.8 | 3.5 |
| | Overall Fold | 0.9 | 1.0 | 3.6 | 3.2 |
| | Change in | | | | |
| | GMT | | | | |
| B.1.351 | GMT | 60 | 128 | 336 | 487 |
| | Fold Change | 1.0 | 2.1 | 5.6 | 8.1 |
| | in GMT | 0.5 | 1.0 | 2.6 | 3.8 |
| | Overall Fold | | | 2.6~5.6 | 3.8~8.1 |
| | Change in | | | | |
| | GMT | | | | |
| B.1.617.2 | GMT | 550 | 876 | 5237 | 3376 |
| | Fold Change | 44 | 56 | 319 | 399 |
| | in GMT | 1.0 | 1.3 | 7.3 | 9.1 |
| | Overall Fold | 0.8 | 1.0 | 5.7 | 7.1 |
| | Change in | | | | |
| | GMT | | | | |
| B.1.1.529 | GMT | 42 | 49 | 164 | 138 |
| (BA.1) | Fold Change | 1.0 | 1.2 | 3.9 | 3.3 |
| | in GMT | 0.9 | 1.0 | 3.3 | 2.8 |
| | Overall Fold | | | 3.3~3.9 | 2.8~3.3 |
| | Change in | | | | |
| | GMT | | | | |

Dose 2 μg

| | | | | | |
|---|---|---|---|---|---|
| D614G | GMT | 238 | 126 | 2826 | 5775 |
| | Fold Change | 453 | 218 | 3565 | 2513 |
| | in GMT | 1.0 | 0.5 | 7.9 | 5.5 |
| | Overall Fold | 2.1 | 1.0 | 16.4 | 11.5 |
| | Change in | | | | |
| | GMT | | | | |
| B.1.351 | GMT | 60 | 86 | 400 | 101 |
| | Fold Change | 1.0 | 1.4 | 6.7 | 1.7 |
| | in GMT | 0.7 | 1.0 | 4.7 | 1.2 |
| | Overall Fold | | | 4.7~6.7 | 1.2~1.7 |
| | Change in | | | | |
| | GMT | | | | |
| B.1.617.2 | GMT | 36 | 34 | 276 | 353 |
| | Fold Change | 1.0 | 0.9 | 7.7 | 9.8 |
| | in GMT | 1.1 | 1.0 | 8.1 | 10.4 |
| | Overall Fold | | | 7.7~8.1 | 9.8~10.4 |
| | Change in | | | | |
| | GMT | | | | |
| B.1.1.529 | GMT | 37 | 35 | 23 | 62 |
| (BA.1) | Fold Change | 1.0 | 0.9 | 0.6 | 1.7 |
| | in GMT | 1.1 | 1.0 | 0.7 | 1.8 |
| | Overall Fold | | | 0.6~0.7 | 1.7~1.8 |
| | Change in | | | | |
| | GMT | | | | |

Dose 0.4 μg

| | | | | | |
|---|---|---|---|---|---|
| D614G | GMT | 268 | 304 | 791 | 722 |
| | Fold of Change | 1.0 | 1.1 | 3.0 | 2.7 |
| | in GMT | 0.9 | 1.0 | 2.6 | 2.4 |
| | Overall Fold | | | 2.6~3.0 | 2.4~2.7 |
| | of Change | | | | |
| | in GMT | | | | |
| B.1.351 | GMT | 35 | 27 | 51 | 85 |
| | Fold of Change | 1.0 | 0.8 | 1.5 | 2.4 |
| | in GMT | 1.3 | 1.0 | 1.9 | 3.1 |
| | Overall Fold | | | 1.5~1.9 | 2.4~3.1 |
| | of Change | | | | |
| | in GMT | | | | |
| B.1.617.2 | GMT | 47 | 52 | 148 | 68 |
| | Fold of Change | 1.0 | 1.1 | 3.1 | 1.4 |
| | in GMT | 0.9 | 1.0 | 2.8 | 1.3 |
| | Overall Fold | | | 2.8~3.1 | 1.3~1.4 |
| | of Change | | | | |
| | in GMT | | | | |
| B.1.1.529 | GMT | 24 | 20 | 44 | 28 |
| (BA.1) | Fold of Change | 1.0 | 0.8 | 1.8 | 1.2 |
| | in GMT | 1.2 | 1.0 | 2.2 | 1.4 |
| | Overall Fold | | | 1.8~2.2 | 1.2~1.4 |
| | of Change | | | | |
| | in GMT | | | | |

TABLE 7
Statistical analyses of geometric mean ID50s at week 6
against SARS-CoV-2 variant pseudoviruses (to FIG. 3)

| Nanoparticle | Non-nanoparticle spike | Pseudovirus | p value |
|---|---|---|---|
| 2 μg S6P-12-HBsAg | 2 μg S6P(1-1206) | D614G | 0.0013 |
| 2 μg S6P-16-HBsAg | 2 μg S6P(1-1206) | | 0.0095 |
| 2 μg S6P-12-HBsAg | 2 μg S2P(1-1206) | B.1.617.2 | 0.0383 |
| | 2 μg S6P(1-1206) | | 0.0312 |
| 10 μg S6P-16-HBsAg | 10 μg S2P(1-1206) | | 0.0172 |
| 2 μg S6P-16-HBsAg | 10 μg S2P(1-1206) | | 0.0324 |
| | 2 μg S2P(1-1206) | | 0.0106 |
| | 2 μg S6P(1-1206) | | 0.0084 |

The statistical analyses shown in Table 7 were performed using the two-way ANOVA test.

### DNA Encoding SARS-CoV-2 S6P—HBsAgs Elicits Durable nAb Responses

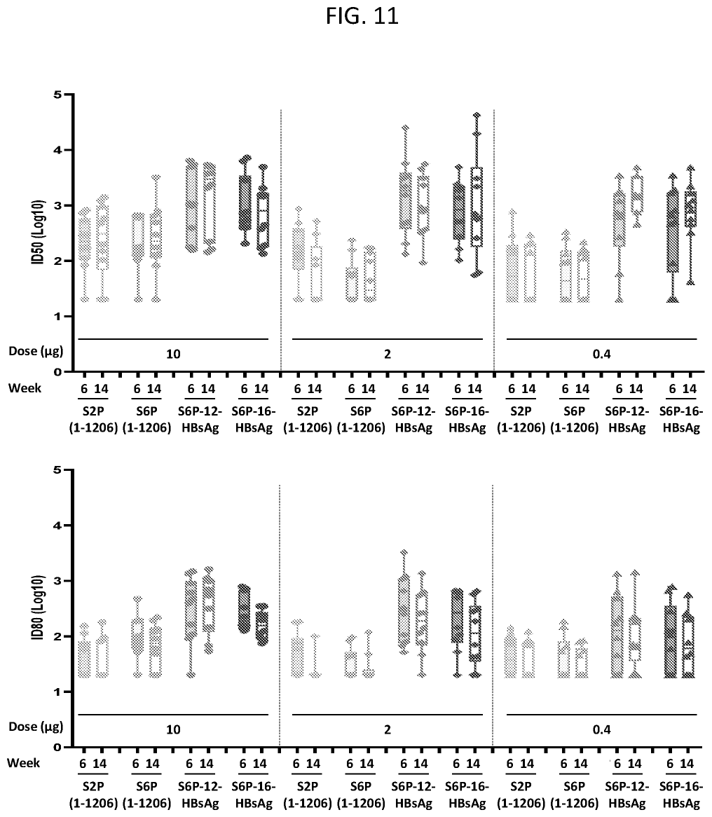

As S6P—HBsAgs elicited potent nAb responses at week 6 against SARS-CoV-2 WA1 and diverse variant pseudoviruses, we assessed the durability of the neutralization potency elicited by S6P—HBsAg. At week 14, the neutralization ID50s and ID80s elicited by S6P-12-HBsAg or S6P-16-HBsAg were maintained at significantly higher levels than those elicited by the same or higher dose of soluble S2P and S6P (FIG. 11). The ID50s and ID80s elicited by these S6P—HBsAgs at week 14 are comparable to those at week 6. Notably, the ID50s elicited by 0.4 μg of S6P-12-HBsAg or S6P-16-HBsAg increased from week 6 to week 14, though without statistical significance.

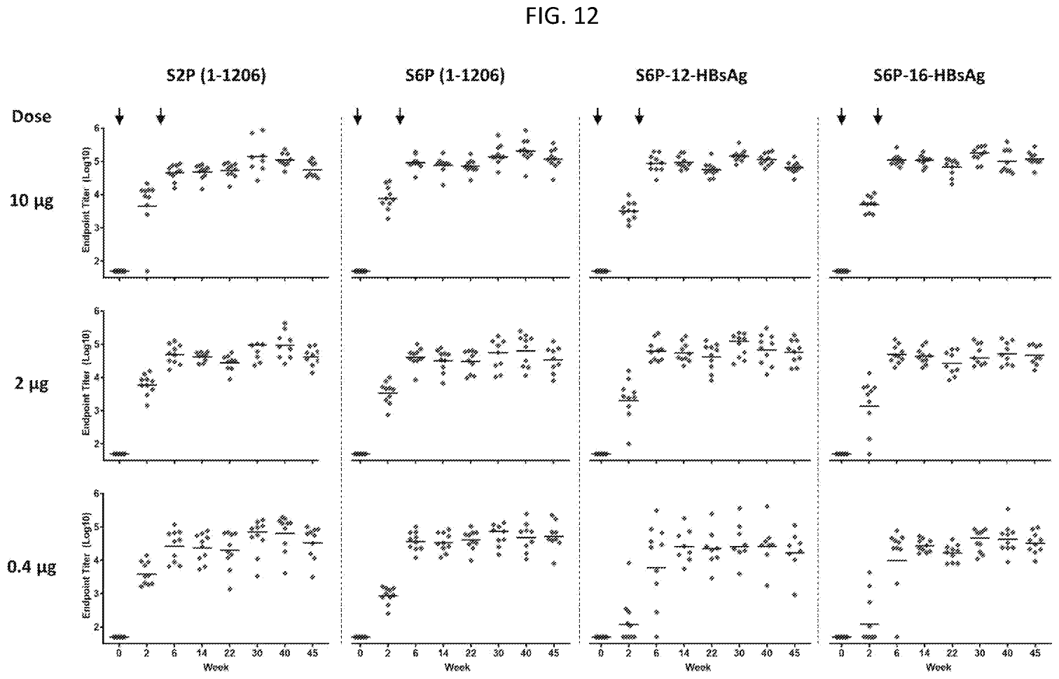

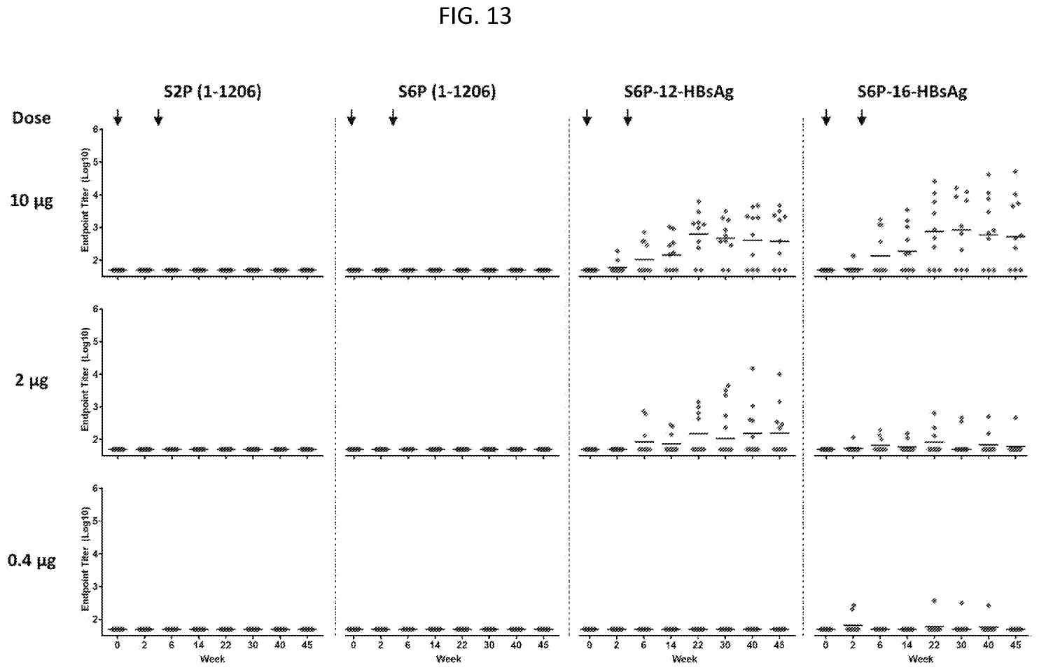

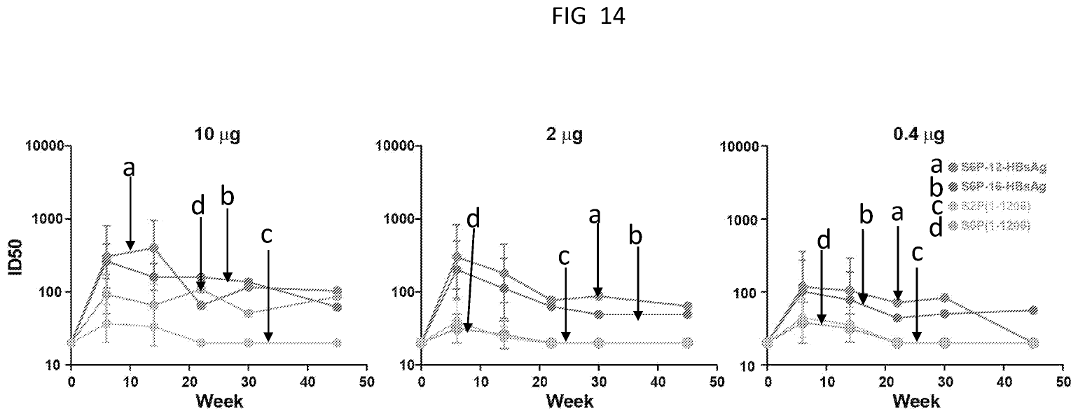

Based on the potent neutralizing antibody response at week 14 elicited by S6P—HBsAg, we further monitored binding antibodies in the animal sera to WA1 S2P and HBsAg from week 0 to week 45. Potent anti-WA1 S2P antibodies were detected from week 6 to week 45, for mice immunized twice with S2P (1-1206), S6P (1-1206), S6P-12-HBsAg and S6P-16-HBsAg at 10, 2 and 0.4 μg doses (FIG. 12). The anti-HBsAg binding antibodies were only detected from week 6 to week 45 in 10 and 2 μg of S6P—HBsAg groups, with significantly lower titers than the anti-S2P antibodies (FIG. 13).

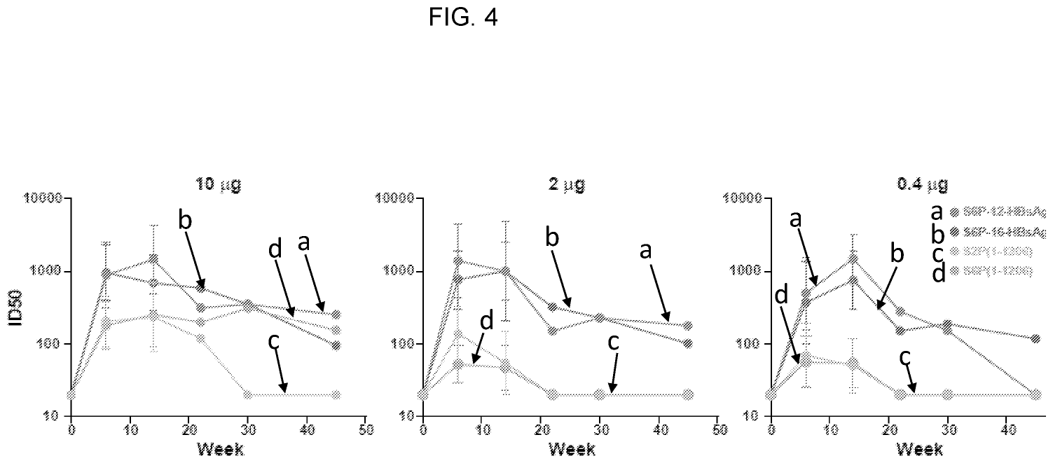

Encouraged by the durable anti-S2P antibody response elicited by S6P—HBsAg, we further tested the neutralization potency of the pooled sera from each group from week 22 to week 45. At 10 μg dose, the neutralization activity was detected up to week 22 for soluble S2P and up to week 45 for soluble S6P and S6P—HBsAg at comparable levels (FIG. 4, 14). While the neutralization activity was only detected up to week 14 for 2 and 0.4 μg soluble S2P and S6P groups, the neutralization activity was maintained at substantially higher levels through week 45 for the same dose of S6P—HBsAg groups, with the only exception been the 0.4 μg S6P-12-HBsAg group which showed no neutralization potency at week 45.

DNA Encoding SARS-CoV-2 S6P—HBsAgs Elicits Greater nAb Responses Against WA1 and B.1.1.529 Pseudoviruses than DNA Encoding S2P (1-1273) or Soluble S6P in Mice Preimmunized with RECOMBIVAX HB®

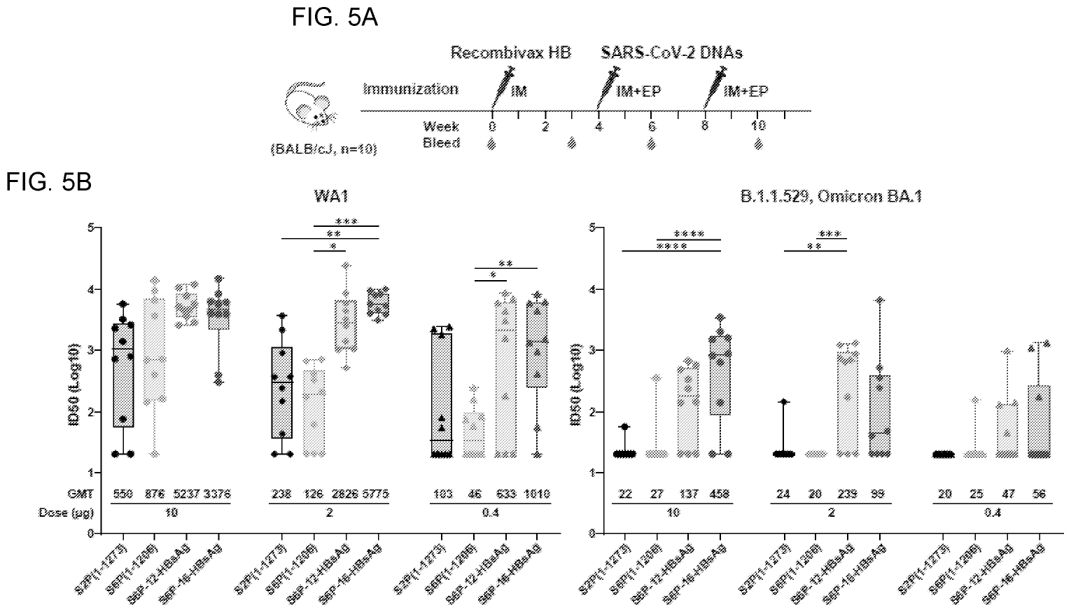

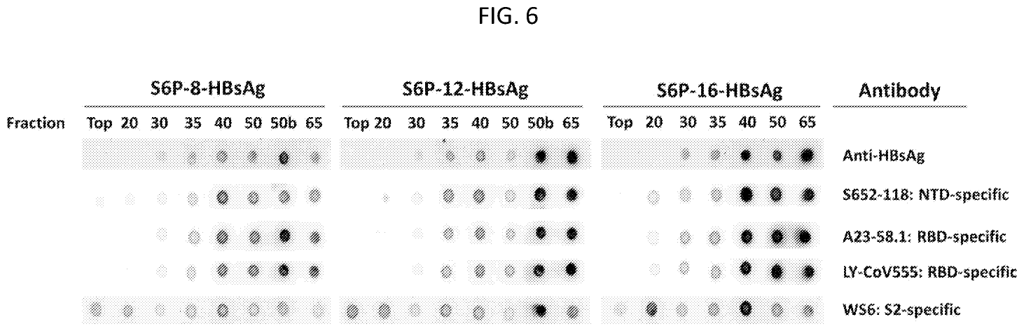

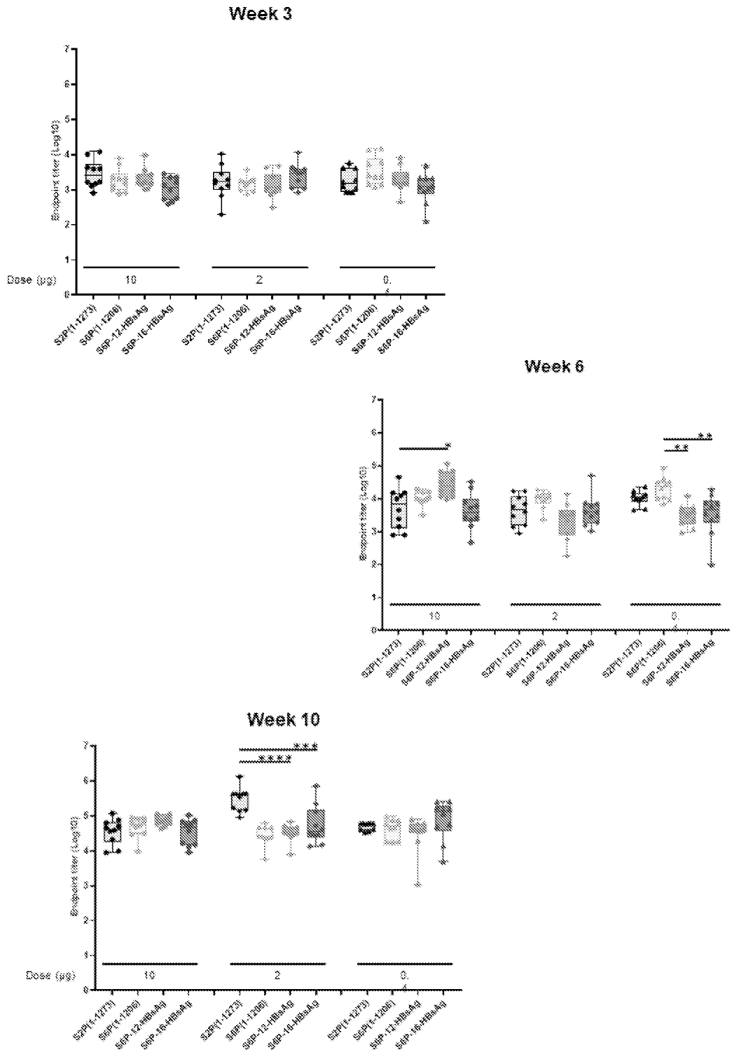

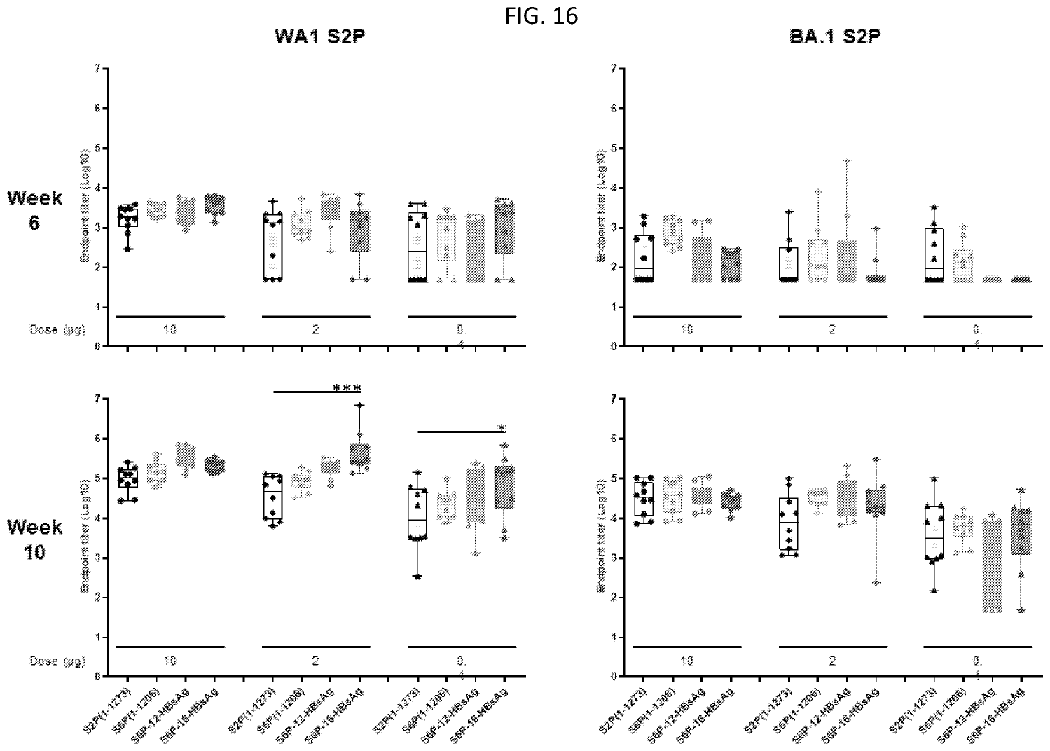

As a large population worldwide has been exposed to HBsAg by either natural infection or hepatitis B vaccination, we tested whether SARS-CoV-2 S6P—HBsAgs can elicit potent antibody responses against SARS-CoV-2 in mice preimmunized with RECOMBIVAX HB®. RECOMBIVAX HB® is a vaccine against hepatitis B virus (HBV) manufactured by Merck. Two DNA immunizations were administered at weeks 4 and 8 to mice preimmunized with RECOMBIVAX HB® at week 0 (FIG. 5A). Potent anti-HBsAg antibodies were detected from all groups of animals at weeks 3, 6 and 10 (FIG. 15). Following DNA immunizations plus electroporation, 10, 2 and 0.4 μg S6P—HBsAg elicited comparable levels of anti-WA1 S2P antibodies at week 6 to those elicited by the same dose of non-nanoparticle forms of S2P (1-1273) and S6P (1-1206) (FIG. 16). At 2 weeks post the 2nd immunization, 2 and 0.4 μg S6P-16-HBsAg elicited significantly higher levels of anti-S2P antibodies than the same dose of S2P (1-1273).

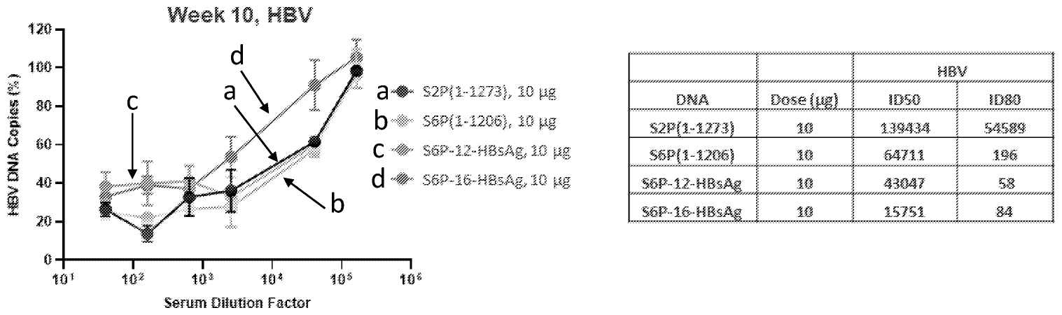

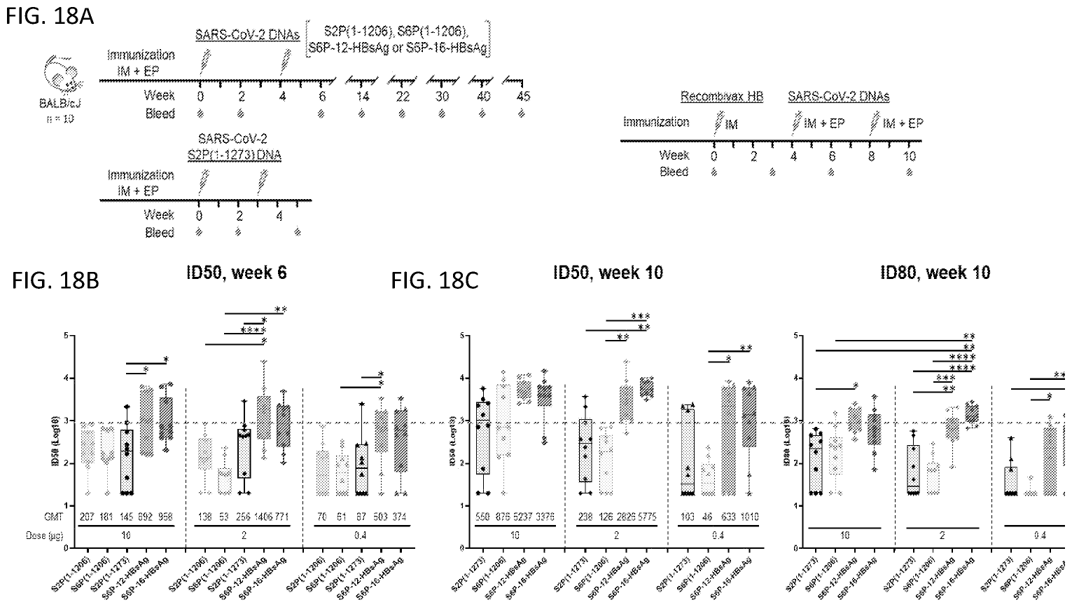

In terms of neutralizing antibody responses, we detected significantly increased WA1 ID50s and ID80s in 2 and 0.4 μg of S6P-12-HBsAg and S6P-16-HBsAg groups than in the same dose of spike groups (FIG. 5B, 17A, Table 8). 0.4 to 10 μg S6P—HBsAgs elicited 6.1˜ 24.3-fold and 3.9˜ 45.8-fold higher geometric mean ID50s than the same dose of S2P (1-1273) and soluble S6P, respectively (Table 9). Between mice without and with RECOMBIVAX HB® pre-vaccination, we observed up to 7.5-fold difference in ID50s at 2 weeks post the second DNA immunization (Table 10). The ID80s at week 10 from mice preimmunized with RECOMBIVAX HB® in the 10 and 2 μg S6P—HBsAg groups were comparable to those at week 6 in mice without RECOMBIVAX HB® preimmunization but immunized with the same immunogens (FIG. 18). Two immunizations of S6P—HBsAgs also elicited potent neutralizing antibody responses against BA.1 pseudovirus, with significantly higher ID50s and ID80s than those elicited by S2P (1-1273) and S6P (1-1206) (FIG. 5B, 17B).

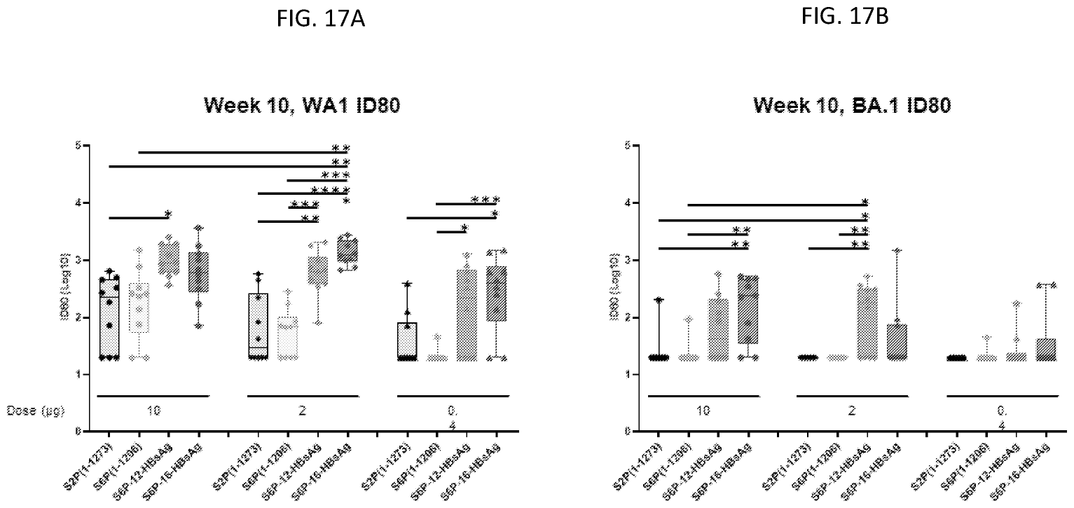

To test whether S6P—HBsAg can also serve as a booster immunization for HBV vaccine, we tested the neutralization potency of the mouse sera against HBV. Neutralization potency against live HBV viruses were only detected in mice preimmunized with RECOMBIVAX HB® followed by two immunizations of 10 μg of SARS-CoV-2 S2P (1-1273). S6P (1-1206). S6P-12-HBsAg or S6P-16-HBsAg DNAs (FIG. 17C). The neutralization potency appeared to be higher in 10 μg S2P (1-1273) and S6P (1-1206) groups than in 10 μg of either S6P—HBsAg group.

TABLE 8
Statistical analyses of geometric mean ID50s at week 10 for mice
preimmunized with RECOMBIVAX HB ® (to FIG. 5)

| Nanoparticle | Non-nanoparticle spike | Pseudovirus | p value |
|---|---|---|---|
| 2 μg S6P-12-HBsAg | 2 μg S6P(1-1206) | WA1 | 0.0054 |
| 0.4 μg S6P-12-HBsAg | 0.4 μg S6P(1-1206) | | 0.0405 |
| 2 μg S6P-16-HBsAg | 2 μg S2P(1-1273) | | 0.0037 |
| | 2 μg S6P(1-1206) | | 0.0002 |
| 0.4 μg S6P-16-HBsAg | 0.4 μg S6P(1-1206) | | 0.0059 |
| 2 μg S6P-12-HBsAg | 10 μg S2P(1-1273) | Omicron BA.1 | 0.0036 |
| | 2 μg S2P(1-1273) | | 0.0034 |
| | 10 μg S6P(1-1206) | | 0.006 |
| | 2 μg S6P(1-1206) | | 0.0009 |
| 10 μg S6P-16-HBsAg | 10 μg S2P(1-1273) | | <0.0001 |
| | 10 μg S6P(1-1206) | | <0.0001 |

The statistical analyses shown in Table 8 were performed using the Two-way ANOVA test.

TABLE 9
Geometric mean neutralization ID50 titers at week 10 in mice
preimmunized with RECOMBIVAX HB ® (to FIG. 5)

| | S2P | S6P | S6P-12- | S6P-16- |
|---|---|---|---|---|
| Immunogen | (1-1273) | (1-1206) | HBsAg | HBsAg |

Dose 10 μg

| | | | | | |
|---|---|---|---|---|---|
| WA1 | GMT | 550 | 876 | 5237 | 3376 |
| | Fold of | 1.0 | 1.6 | 9.5 | 6.1 |
| | Change | 0.6 | 1.0 | 6.0 | 3.9 |
| | in GMT | | | | |
| | Overall Fold | | | 6.0~9.5 | 3.9~6.1 |
| | of Change | | | | |
| | in GMT | | | | |
| B.1.1.529 | GMT | 22 | 27 | 137 | 458 |
| (BA.1) | Fold of | 1.0 | 1.2 | 6.2 | 20.8 |
| | Change | 0.8 | 1.0 | 5.1 | 17.0 |
| | in GMT | | | | |
| | Overall Fold | | | 5.1~6.2 | 17.0~20.8 |
| | of Change | | | | |
| | in GMT | | | | |

Dose 2 μg

| | | | | | |
|---|---|---|---|---|---|
| WA1 | GMT | 238 | 126 | 2826 | 5775 |
| | Fold of | 1.0 | 0.5 | 11.9 | 24.3 |
| | Change | 1.9 | 1.0 | 22.4 | 45.8 |
| | in GMT | | | | |
| | Overall Fold | | | 11.9~22.4 | 24.3~45.8 |
| | of Change | | | | |
| | in GMT | | | | |
| B.1.1.529 | GMT | 24 | 20 | 239 | 99 |
| (BA.1) | Fold of | 1.0 | 0.8 | 10.0 | 4.1 |
| | Change | 1.2 | 1.0 | 12.0 | 5.0 |
| | in GMT | | | | |
| | Overall Fold | | | 10.0~12.0 | 4.1~5.0 |
| | of Change | | | | |
| | in GMT | | | | |

Dose 0.4 μg

| | | | | | |
|---|---|---|---|---|---|
| WA1 | GMT | 103 | 46 | 633 | 1010 |
| | Fold of | 1.0 | 0.4 | 6.1 | 9.8 |
| | Change | 2.2 | 1.0 | 13.8 | 22.0 |
| | in GMT | | | | |
| | Overall Fold | | | 6.1~13.8 | 9.8~22.0 |
| | of Change | | | | |
| | in GMT | | | | |
| B.1.1.529 | GMT | 20 | 25 | 47 | 56 |
| (BA.1) | Fold of | 1.0 | 1.3 | 2.4 | 2.8 |
| | Change | 0.8 | 1.0 | 1.9 | 2.2 |
| | in GMT | | | | |
| | Overall Fold | | | 1.9~2.4 | 2.2~2.8 |
| | of Change | | | | |
| | in GMT | | | | |

In Table 9, GMT: Geometric mean titer. Fold of change was calculated in comparison with the ID50 against the same pseudovirus elicited by the same dose of non-nanoparticle form of spike.

TABLE 10
Comparison of ID50s between mice with and without RECOMBIVAX HB ® preimmunization.

| | | Geometric mean ID50 against WA1 | | | Geometric mean ID50 against BA. 1 | | |
|---|---|---|---|---|---|---|---|
| Prime & | | RECOMBIVAX | RECOMBIVAX | | RECOMBIVAX | RECOMBIVAX | |
| boost | Dose | HB ® − | HB ® + | Ratio | HB ® − | HB ® + | Ratio |
| immunogen | (μg) | Week 6 | Week 10 | HB⁺/HB⁻ | Week 6 | Week 10 | HB⁺/HB⁻ |
| S6P | 10 | 181 | 876 | 4.8 | 49 | 27 | 0.6 |
| (1-1206) | 2 | 53 | 126 | 2.4 | 35 | 20 | 0.6 |
| | 0.4 | 61 | 46 | 0.8 | 20 | 25 | 1.3 |
| S6P-12- | 10 | 892 | 5237 | 5.9 | 164 | 137 | 0.8 |
| HBsAg | 2 | 1406 | 2826 | 2.0 | 23 | 239 | 10.4 |
| | 0.4 | 503 | 633 | 1.3 | 44 | 47 | 1.1 |
| S6P-16- | 10 | 958 | 3376 | 3.5 | 138 | 458 | 3.3 |
| HBsAg | 2 | 771 | 5775 | 7.5 | 62 | 99 | 1.6 |
| | 0.4 | 374 | 1010 | 2.7 | 28 | 56 | 2.0 |
| S2P | 10 | 145 | 550 | 3.8 | 20 | 22 | 1.1 |
| (1-1273) | 2 | 256 | 238 | 0.9 | 21.8 | 24 | 1.1 |
| | 0.4 | 87 | 103 | 1.2 | 20 | 20 | 1.0 |

### DISCUSSION

In this study, we designed and characterized self-assembling SARS-CoV-2 spike-HBsAg nanoparticles and assessed their immunogenicity in mice via genetic delivery. We show that these HBsAg nanoparticles displaying SARS-CoV-2 S6P elicit potent, broad and durable immune responses compared to non-nanoparticle form of stabilized SARS-CoV-2 spikes. Additionally, preimmunization with an HBV vaccine leads to a further enhancement of the HBsAg-nanoparticle-elicited immune responses against SARS-CoV-2 and its variants.

SARS-CoV-2 spike exhibits a metastable prefusion conformation that spontaneously transitions to its post-fusion conformation. The two-proline-stabilized SARS-CoV-2 spike S2P in its prefusion conformation has been used in the current COVID-19 vaccines from Moderna, Pfizer-BioNTech and Johnson & Johnson. We have screened WA1 spike, S2P and S6P for nanoparticle formation on a HBsAg core. S6P succeeded to form well-defined HBsAg nanoparticles whereas small fractions of wild type spike and S2P formed nanoparticles. This may be due to the metastability of SARS-CoV-2 spike. S2P is not as stable as S6P, as four proline substitutions in S6P in addition to the K986P and V987P substitutions further stabilize its prefusion conformation. Both S6P-12-HBsAg and S6P-16-HBsAg bound well to human ACE2 and mAbs specific to the RBD or NTD domain of SARS-CoV-2 spike, suggesting the accessibility of the neutralizing epitopes in the S6P—HBsAg nanoparticles.

DNA encoding S6P-12-HBsAg and S6P-16-HBsAg elicited potent nAb responses to SARS-CoV-2 and its variants. The lower binding antibody levels but higher nAb responses elicited by 0.4 μg S6P—HBsAg than those elicited by the same dose of soluble S2P and S6P are indicative of the higher immunogenicity of S6P—HBsAg. In addition, the nAb responses elicited by S6P—HBsAg persisted through week 45 and is more durable than those elicited by soluble S2P and S6P via genetic delivery.

The neutralization potency induced by DNA delivery of S6P—HBsAgs is comparable to that elicited by intramuscular delivery of 1 μg mRNA-1273 or SARS-CoV-2 S2P protein. Using the same route of genetic delivery, S6P—HBsAgs elicited substantially higher neutralization potency than S2P (1-1273), whose coding sequence matches that in mRNA-1273. Similar observations were noted in mice preimmunized with an HBV vaccine, with further significantly enhanced potency. The higher neutralization potency elicited by S6P—HBsAgs than by non-nanoparticle forms of stabilized spike alone could be contributed by several factors. First, S6P—HBsAg nanoparticles with diameters of ˜ 70 nm are much larger than the SARS-CoV-2 spike. The large size of these nanoparticles may result in more efficient internalization of the spike by antigen presenting cells (APCs) and retention of the spike on lymph node follicles. The repetitive array of the spikes on S6P—HBsAg nanoparticles may also enable efficient binding and activation of multiple B cell receptors. In addition, the antibody responses elicited by S6P—HBsAgs were mainly directed against the spikes displayed on the HBsAg nanoparticles as opposed to HBsAg, suggesting efficient presentation of the spike on the surface of these nanoparticles by the HBsAg core.

Two immunizations of S6P—HBsAgs elicited robust neutralization potency against B.1.1.529 (Omicron BA.1) pseudovirus, in mice preimmunized with RECOMBIVAX HB®. While a 3rd dose of mRNA vaccines is needed to offer better protection against Omicron variants (Tseng et al., Nat Med, 28, 1063-1071, 2022; Lauring et al., BMJ, 2022. 376 (e069761)) and a fourth dose retains low efficacy in preventing Omicron infections (Regev-Yochay et al., medRxiv, 2022.02.15.22270948), and the bivalent COVID-19 vaccines do not resolve the durability issue, S6P—HBsAgs may provide a strategy to offer longer and better protection against SARS-CoV-2 in populations that have previously exposed to HBsAg by infection or vaccination. As per WHO estimates, approximately ⅓ of the global population have been infected with HBV in 2010. The immunization rate with 3 doses of HBV vaccine during infancy has also reached 85% worldwide in 2019. This high rate of exposure to HBsAg in global population further highlights the potential of SARS-CoV-2 S6P—HBsAg as a genetic vaccine platform for inducing more potent and durable protection against SARS-CoV-2.

Many nanoparticle designs require an additional protein conjugation step, which does not allow them to be amenable to gene-based delivery. Our S6P—HBsAg designs exhibit advantages as genetic vaccine candidates over similar nanoparticle vaccine designs utilizing an additional conjugation step. Further, the utilization of spike in our designs enables more neutralizing epitopes than RBD-based vaccine designs.

In conclusion, SARS-CoV-2 S6P—HBsAg can elicit more potent and durable nAb responses against diverse SARS-CoV-2 strains than non-nanoparticle form of stabilized spikes, including SARS-CoV-2 full-length S2P with its coding sequence matching that in mRNA-1273. The nAb responses elicited by S6P—HBsAg can persist 7 months longer than soluble stabilized spikes and were enhanced by pre-exposure to HBsAg.

### Methods

### DNA Construct Design

The HBsAg nanoparticles displaying SARS-CoV-2 spike, S2P or S6P were designed as spike-HBsAg fusion proteins. The coding sequence of the ectodomain (aa 1-1206) of SARS-CoV-2 WA1 spike (NC_045512.2), 2P- or 6P-stabilized mutant (S2P or S6P) (Wrapp et al., Science, 2020. 367 (6483): p. 4; Hsieh et al., Science, 2020. 369 (6510): p. 5.) was joined with the coding sequence of HBsAg (aa 1-226, AET06188.1). The linker was made up of varying repeats of GS dipeptide. The coding sequences of SARS-CoV-2 spike-HBsAgs were inserted into the CMVR8400 vector via XbaI and BamHI sites. The ectodomain of SARS-CoV-2 S2P or S6P, as well as the full-length S2P (aa 1-1273) were also constructed in the CMVR8400 vector via the same restriction sites as above, respectively. Each construct was human codon-optimized, synthesized and confirmed by DNA sequencing.

### Protein Expression and Purification

The SARS-CoV-2 spike-HBsAg plasmids were transfected into Expi293F cells using ExpiFectamine 293 transfection kit following the manufacturer's instructions. The transfected culture was grown at 37° C. for 5 days before harvest. The culture was then spun down at 10,000×g at 20° C. for 30 min. The supernatant was collected and passed through a 0.2 μm filter. The supernatant was further spun through 20% sucrose cushion in a buffer containing 20 mM MES, 150 mM NaCl, pH 6.0. The ultracentrifugation was performed with a Surespin rotor at 20,000 rpm, 4° C. for 2 hours. The pellet was resuspended in a buffer containing 20 mM MES, 150 mM NaCl, pH 6.0, filtered through a 0.45 μm filter, and then spun through a sucrose gradient consisting of 1.5 ml of 20% to 65% sucrose in a buffer containing 20 mM MES, 150 mM NaCl, pH 6.0. This step of ultracentrifugation was done with a Th-641 rotor at 36,000 rpm, 4° C. for 8 hours. The sucrose fractions were collected and stored at 4° C. A dialysis against PBS buffer, pH 7.4, was performed before use. SARS-CoV-2 WA1, B.1.351, B.1.617.2 and B.1.1.529 S2Ps, and WA1 S6P were constructed and prepared as previously described (Wrapp et al., Science, 2020. 367 (6483): p. 4; Hsieh et al., Science, 2020. 369 (6510): p. 5). The tags in S2P and S6P were cleaved off and further purified by sizing column purification. Human ACE2, SARS-CoV-2 RBD, NTD recombinant proteins were purified as described (Corbett et al., Nature, 2020. 586 (7830): p. 5; Zhou et al., Cell Host Microbe, 2020. 28 (6): p. 13). The purified proteins were aliquoted, frozen in liquid nitrogen, and kept at −80° C. before use.

### Negative-Stain Electron Microscopy

The sucrose fractions of SARS-CoV-2 spike-HBsAg fusion proteins were buffer exchanged to PBS containing 5-10% sucrose, and then applied to a freshly glow-discharged carbon-coated grid for about 15 s. The grid was washed with buffer containing 10 mM HEPES, pH 7.0, and 150 mM NaCl, followed by negative staining with 0.7% uranyl formate. Images were collected at a nominal magnification of 57,000 using EPU software on a Thermo Scientific Talos F200C electron microscope operated at 200 kV and equipped with a 4 k×4 k Ceta CCD camera or at a nominal magnification of 50,000 using SerialEM (DN, M., J Struct Biol, 2005. 152 (1): p. 16) on an FEI T20 electron microscope operated at 200 kV and equipped with an Eagle CCD camera. The corresponding pixel sizes were 0.253 and 0.22 nm. Particles were picked using in-house written software (YT, unpublished) or e2boxer from the EMAN2 software package (Tang G, P. L., Baldwin P R, Mann D S, Jiang W, Rees I, Ludtke S J., EMAN2: an extensible image processing suite for electron microscopy. J Struct Biol, 2007. 157 (1): p. 9). Reference-free 2D classification was performed using Relion (S H, S., RELION: implementation of a Bayesian approach to cryo-EM structure determination. J Struct Biol, 2012. 180 (3): p. 12).

### ELISA to Assess ACE2-Binding and Antigenicity of SARS-CoV-2 Spike—HBsAgs

ELISA plates (Thermo Fisher, 442404) were coated with 1 μg/ml of SARS-CoV-2 WA1 or variant S2P, WA1 S6P or S6P—HBsAg in PBS buffer, pH 7.4, 100 μl/well at 4° C. for 16 hours (Wrapp et al., Science, 2020. 367 (6483): p. 4). The plates were washed with PBST thrice, 300 μl per well per time and then blocked with 5% skim milk in 1× PBST at room temperature for 1 hour. The binding of SARS-CoV-2 WA1 S2P, S6P and spike-HBsAg was done with 4 μg/ml to 10 μg/ml SARS-CoV-2 mAbs (Table 3) or with 6.1 ng/ml to 100 μg/ml human ACE2 (Zhou et al., Cell Host Microbe, 2020. 28 (6): p. 13). The goat anti-human IgG Fc-HRP antibody (Invitrogen, A18817, 1/5000) was used to detect the ACE2 binding. The primary antibody incubation was done at room temperature for 30 min. Following three washes, the plates were incubated with HRP-conjugated anti-human or anti-mouse antibody ( 1/2000, Thermo Fisher) at room temperature for 30 min. The plates were then washed thrice and developed with 3,5,3′,5′-tetramethylbenzidine (TMB) (KPL) at room temperature for 10 min. After quenched with IN H₂SO₄ (Fisher), the plates were read at 450 nm with a SpectraMax Plus 384 microplate reader. The data were plotted and analyzed with GraphPad Prism.

### Immunogenicity Evaluation in Mice

The immunogenicity studies in 6- to 8-week-old female BALB/cJ mice (Jackson Laboratory) were performed in compliance with all pertinent US NIH regulations and approval from the Animal Care and Use Committee (ACUC) of the Vaccine Research Center (VRC). Pre-bleeds were collected the day before the first immunization. Plasmid DNAs encoding SARS-CoV-2 WA1 S2P (1-1206), S6P (1-1206), S6P-12-HBsAg, S6P-16-HBsAg, S2P (1-1273) were diluted prior to immunization in PBS, pH 7.4. Naïve mice were injected intramuscularly twice spaced 4-week apart in both hind legs with a total of 10, 2 or 0.4 μg plasmid DNA in 100 μl PBS, pH 7.4. S2P (1-1273) was administered twice with a 3-week interval to match the immunization regimen of BNT162b2. Mice preimmunized with 1 μg RECOMBIVAX HB® vaccine intramuscularly (Davis et al., J Immunol, 1998. 160 (2): p. 7) at week 0 received two immunizations of 10, 2 or 0.4 μg SARS-CoV-2 WA1 S2P (1-1273), S6P (1-1206), S6P-12-HBsAg and S6P-16-HBsAg DNA at week 4 and week 8, respectively. Electroporation was done following each DNA immunization with the AgilePulse System (Harvard Apparatus) using manufacturer-recommended setting (Dowd et al., Science, 2016. 354 (6309): p. 4). The electroporation was applied to the muscles at the injection sites in both hind legs. Mouse sera were collected 2 weeks post each DNA immunization, and 3 weeks post RECOMBIVAX HB® vaccination (Davis et al., J Immunol, 1998. 160 (2): p. 7).

### Serum ELISA

ELISA plates (Thermo Fisher, 442404) were coated with 1 μg/ml of SARS-CoV-2 WA1, variant S2P, or HBsAg (ProspecBio, HBS-875) in PBS, pH 7.4 at 4° C. for 16 hours (Corbett et al., Nature, 2020. 586 (7830): p. 5; Zhou et al., Cell Host Microbe, 2020. 28 (6): p. 13). Standard washes and blocking steps were done as described above. The plates were blocked for 2 hours. The sera were diluted by 100-fold in 5% skim milk in PBST. A serial 4-fold dilution for weeks 0 and 2 sera or 6-fold dilution for week 6 sera was applied to the 100-fold dilution preparations. The plates were incubated with diluted sera at room temperature for 1 hour. The HRP-conjugated anti-mouse secondary antibody (Thermo Fisher, 1/2000) was used to detect the antibody responses. The endpoint titers were calculated as the dilution that yielded an optical density equivalent to 4×background (secondary antibody alone).

### Pseudovirus Neutralization Assay

The codon-optimized SARS-CoV-2 spike (Wuhan-1, GenBank: MN908947.3; D614G, B.1.351, B.1.617.2, B.1.1.529) plasmids were used (Wang et al., Science, 2021. 373 (6556)). Pseudoviruses were generated by co-transfection of transducing plasmid pHR′ CMV-Luc encoding a luciferase reporter, lentivirus packaging plasmid pCMVd8.2, a TMPRSS2 plasmid and a spike plasmid of SARS-CoV-2 or variants into HEK293T/17 cells (ATCC CRL-11268) using Lipofectamine 3000 transfection reagent (Thermo Fisher, L3000-001); (Corbett et al., Nature, 2020. 586 (7830): p. 5; Zhou et al., Cell Host Microbe, 2020. 28 (6): p. 13; Wang et al., Science, 2021. 373 (6556); Zhou et al., Science, 376 (6591), 2022. (DOI: 10.1126/science.abn8897)). Heat-inactivated serum was mixed with the titrated pseudoviruses, incubated, and then added to pre-plated 293T-ACE2 cells (from Dr. Michael Farzan) in triplicate. Following 2 hours of incubation, wells were replenished with 150 μl of fresh media. 72 hours later, the cells were lysed and the luciferase activity was recorded in relative light units (RLU). The neutralization activity was normalized to uninfected cells as 100% neutralization and to cells infected with only pseudovirus as 0% neutralization. ID50 and ID80 titers were determined using a log (agonist) vs normalized response (variable slope) nonlinear function in GraphPad Prism.

### HBV Neutralization Assay

Group pooled sera from mice immunized with 10 μg of SARS-CoV-2 DNAs were tested for neutralization potency against live HBV viruses (subtype ayw, ImQuest BioSciences). Week 22 sera from mice without RECOMBIVAX HB® preimmunization and week 10 sera from mice with RECOMBIVAX HB® preimmunization were evaluated. HepG2-NTCP cells (Baruch S. Blumberg Institute) were seeded in a 48-well plate and incubated for 24 hours in DMEM F12 (Gibco, 11320-030) supplemented with 10% FBS, 1% Fungizone and 2 μg/mL Puromycin. Pooled sera were diluted in the medium with a 4-fold series of 7 dilutions. The diluted sera were incubated with HBV viruses (MOI: 1000) for 45 minutes at 37° C. with 5% CO₂. Following the incubation, media was removed from the pre-seeded plates. The serum/virus samples were added in triplicate to the cells and incubated for 18 to 24 hours. A neutralizing mAb against HBV H015 (Acrobiosystems, HBG-M406-50 μg) up to 1 μg/ml and Tenofovir disoproxil fumarate (TDF, Gilead) up to 1 μM were used as positive controls. The virus and compound were then removed by 5 washes and replaced with fresh media with and without H015 or TDF. This was repeated on day 4 and day 7 post infection. On day 10 the supernatant was collected and evaluated by qPCR assay. The primers and probe were from IDT and listed as follows: HBV-AD38-qF1: 5′-CCGTCTGTGCCTTCTCATCTG-3′ (SEQ ID NO: 370); HBV-AD38-qR1: 5′-AGTCCAAGAGTCCTCTTATACAAGACC-3′ (SEQ ID NO: 371); and HBV-AD38-qP1: 5′-FAM/CCGTGTGCA/ZEN/CTTCGCTTCAC-3′ BHQ1 (SEQ ID NO: 372).

In view of the many possible aspects to which the principles of the disclosed subject matter may be applied, it should be recognized that the illustrated aspects are only preferred examples of the disclosure and should not be taken as limiting the scope of the disclosure. Rather, the scope of the disclosure is defined by the following claims. We therefore claim all that comes within the scope and spirit of these claims.

1. A nucleic acid molecule encoding a fusion protein comprising:
a recombinant SARS-CoV-2 spike(S) ectodomain comprising F817P, A892P, A899P, A942P, K986P, and V987P substitutions and an amino acid sequence at least 90% identical to residues 14-1206 of SEQ ID NO: 2 fused via a peptide linker to a hepatitis B surface antigen (HBsAg) protein.

2. The nucleic acid molecule of claim 1, wherein the fusion protein self-assembles when expressed in mammalian cells to form a HBsAg nanoparticle with SARS-CoV-2 S ectodomain trimers extending radially outward from an outer surface of the HBsAg nanoparticle.

3. The nucleic acid molecule of claim 1, wherein a S1/S2 protease cleavage site of the SARS-CoV-2 S ectodomain is mutated to inhibit protease cleavage.

4. The nucleic acid molecule of claim 1, wherein the S1/S2 protease cleavage site is mutated by a RRAR (682-685)-to-GSAS substitution.

5. The nucleic acid molecule of claim 1, wherein the SARS-CoV-2 S ectodomain comprises one or more of: K417N, L452R, T478K, E484K, E484Q, N501Y, and P681R substitutions.

6. The nucleic acid molecule of claim 1, wherein the SARS-CoV-2 S ectodomain comprises one of a N501Y substitution;
a E484K substitution;
N501Y, K417N, and E484K substitutions;
L452R, T478K, and P681R substitutions; or
L452R, E484Q, and P681R substitutions.

7. The nucleic acid molecule of claim 1, wherein the SARS-CoV-2 S ectodomain comprises the F817P, A892P, A899P, A942P, K986P, V987P, and RRAR (682-685)-to-GSAS substitutions and an amino acid sequence at least 90% identical, at least 95% identical, at least 99% identical, or identical, to:
residues 14-1206 of SEQ ID NOs: 3, 19 or 20;
residues 14-1205 of SEQ ID NOs: 21-23 or 30;
residues 14-1208 of SEQ ID NOs: 16, 24-25, 28-29, 31-32, 34-36, 38-42, or 44;
residues 14-1201 of SEQ ID NO: 26;
residues 14-1200 of SEQ ID NO: 27;
residues 14-1203 of SEQ ID NO: 37; or
residues 14-1207 of SEQ ID NOs: 33 and 43.

8. The nucleic acid molecule of claim 1, wherein the SARS-CoV-2 S ectodomain comprises the F817P, A892P, A899P, A942P, K986P, V987P, and RRAR (682-685)-to-GSAS substitutions and an amino acid sequence at least 90% identical, at least 95% identical, at least 99% identical, or identical to residues 14-1206 of SEQ ID NO: 3.

9. The nucleic acid molecule of claim 1, wherein the peptide linker is from 12-39 amino acid residues in length.

10. The nucleic acid molecule of claim 9, wherein the peptide linker is from 12-32 residues in length.

11. The nucleic acid molecule of claim 9, wherein the peptide linker is 24 or 32 residues in length.

12. The nucleic acid molecule of claim 1, wherein the peptide linker is a glycine-serine linker.

13. The nucleic acid molecule of claim 12, wherein the glycine-serine peptide linker is a repeat of glycine-serine dipeptides.

14. The nucleic acid molecule of claim 1, wherein the sequence of the peptide linker is set forth as any one of SEQ ID NOs: 4-7.

15. The nucleic acid molecule of claim 1, wherein the HBsAg protein comprises or consists of a HBsAg S protein, a HBsAg M protein, or a HBsAg L protein.

16. (canceled)

17. The nucleic acid molecule of claim 15, wherein the HBsAg protein comprises or consists of an amino acid sequence at least 90% identical, at least 95% identical, at least 99% identical, or identical, to any one of SEQ ID NOs: 9 or 45-56.

18. (canceled)

19. The nucleic acid molecule of claim 1, wherein the fusion protein comprises or consists of an amino acid sequence at least 90% identical, at least 95% identical, at least 99% identical, or identical, to residues 14 to the C-terminus of any one of SEQ ID NOs: 10-12, 17-18, 57-296, or 315-321.

20. (canceled)

21. The nucleic acid molecule of claim 1, wherein the nucleic acid molecule is DNA or RNA.

22. The nucleic acid molecule of claim 1, comprising or consisting of:
A DNA sequence at least 80%, at least 90%, or at least 95% identical, or identical, to SEQ ID NO: 13 that encodes a fusion protein set forth as SEQ ID NO: 10, or the complement thereof, or a corresponding RNA sequence or the complement thereof;
a DNA sequence at least 80%, at least 90%, or at least 95% identical, or identical, to SEQ ID NO: 14 that encodes a fusion protein set forth as SEQ ID NO: 11, or the complement thereof, or a corresponding RNA sequence or the complement thereof; or
a DNA sequence at least 80%, at least 90%, or at least 95% identical, or identical, to SEQ ID NO: 15 that encodes a fusion protein set forth as SEQ ID NO: 12, or the complement thereof, or a corresponding RNA sequence or the complement thereof.

23. (canceled)

24. The nucleic acid molecule of claim 1, wherein the nucleic acid molecule is an mRNA or circular RNA with or without modified bases, with or without replication competence.

25. A vector comprising the nucleic acid molecule of claim 1.

26. The vector of claim 25, wherein the vector is a DNA vector, an RNA vector, or a viral vector.

27. A HBsAg protein nanoparticle encoded by the nucleic acid or vector of claim 1.

28. The HBsAg protein nanoparticle of claim 27, comprising SARS-CoV-2 S ectodomain trimers extending radially outward from an outer surface of the nanoparticle.

29. An immunogenic composition comprising the nucleic acid molecule, vector, or HBsAg protein nanoparticle of claim 1 and a pharmaceutically acceptable carrier.

30. The composition of claim 29, wherein the nucleic acid molecule is an mRNA or circular RNA and the composition further comprises a lipid nanoparticle.

31. A method, comprising administering to a subject an amount of the nucleic acid molecule, the vector, HBsAg protein nanoparticle, or the composition of claim 1 in an amount effective to elicit a neutralizing immune response to SARS-CoV-2 in the subject.

32. The method of claim 31, wherein the subject was previously exposed to HBsAg by hepatitis B infection or vaccination.

33. The method of claim 31, wherein the immune response inhibits or prevents SARS-CoV-2 infection, and/or severe COVID19 disease, in the subject.

34. (canceled)
